(19) United States
(12) Patent Application Publication
(10) Pub. No.: US 2025/0353986 A1
(43) Pub. Date: Nov. 20, 2025

(54) COMPOSITIONS, RELATED SYSTEMS AND ARTICLES, AND METHODS OF MAKING AND USING THE SAME

(71) Applicant: CAMBRIDGE ADVANCED HOLDINGS LTD, Grand Cayman (KY)

(72) Inventor: Ali Reza Kamali, Cambridge (GB)

(21) Appl. No.: 18/866,620

(22) PCT Filed: May 19, 2023
(86) PCT No.: PCT/EP2023/063482

§ 371 Date: Nov. 18, 2024

Related U.S. Application Data
(60) Provisional application No. 63/344,349, filed on May 20, 2022.
(60) Provisional application No. 63/398,459, filed on Aug. 16, 2022.
(60) Provisional application No. 63/439,688, filed on Jan. 18, 2023.

(51) Int. Cl. C08J 11/16; C07F 1/02; C07F 1/04; C07F 1/06; C07F 3/06

(52) U.S. Cl. CPC C08J 11/16; C07F 1/02; C07F 1/04; C07F 1/06; C07F 3/06; C07B 2200/13

(57) ABSTRACT

The disclosure relates to various compositions, related systems and articles, and methods of making and using the same. In some aspects, the disclosure relates to compositions containing a nanostructured organic compound, compositions containing an organic compound and a metal-organic framework embedded within the organic compound, and compositions containing an organic compound that is at least partially crystalline and a crystalline metal oxide distributed within the organic compound, as well as related methods of making (e.g., methods of depolymerizing polymers), methods of use (e.g., energy storage, contamination removal), articles (e.g., electrodes), and systems (e.g., energy storage systems, systems containing such energy storage systems) from the compositions of the disclosure. In some aspects, the disclosure relates to a composition that includes a silicon-containing material and a polymer made of imide monomers, as well as related systems and articles, and methods of making and using the same.

### CROSS-REFERENCE TO RELATED APPLICATIONS

This application claims the benefit of the following U.S. patent applications: U.S. Ser. No. 63/344,349, filed May 20, 2022 and entitled “Method of Producing Nanostructured Materials;” U.S. Ser. No. 63/398,459, filed Aug. 16, 2022 and entitled “Compositions Containing an Organic Compound and Crystalline Metal Oxide and Methods of Making the Same;” and U.S. Ser. No. 63/439,688, filed Jan. 18, 2023 and entitled “Method of Converting Waste Plastics Into Nanostructured Monomers and Related Compounds.” The entire disclosure of each of these applications is incorporated by reference herein.

### FIELD

The disclosure relates to various compositions, related systems and articles, and methods of making and using the same. In some aspects, the disclosure relates to compositions containing a nanostructured organic compound, compositions containing an organic compound and a metal-organic framework embedded within the organic compound, and compositions containing an organic compound that is at least partially crystalline and a crystalline metal oxide distributed within the organic compound, as well as related methods of making (e.g., methods of depolymerizing polymers), methods of use (e.g., energy storage, contamination removal), articles (e.g., electrodes), and systems (e.g., energy storage systems, systems containing such energy storage systems) from the compositions of the disclosure. In some aspects, the disclosure relates to a composition that includes a silicon-containing material and a polymer made of imide monomers, as well as related systems and articles, and methods of making and using the same.

### BACKGROUND

Many plastics are produced and consumed worldwide in large quantities. As an example, it has been reported that there is an annual consumption of around 30 million tons of polyethylene terephthalate (“PET”) for the preparation of various products, such as around 400 billion drink bottles. Typically, such products are wasted after single use, due to the lack of relatively simple and effective recycling methods. This contributes to pollution in terrestrial and aquatic environments.

Known methods for chemically depolymerizing waste PET into its monomers often involves using concentrated acids or alkaline solutions at relatively high temperatures using pressurized vessels. Certain other PET recycling methods include using enzymes that can involve prolonged processing periods due to the relatively slow kinetics of the process. In many cases, current polymer recycling methods also produce a mixture of different monomers that require an extra separation purification step.

### SUMMARY

The technology disclosed herein can be used to reduce the amount of waste plastic material while also providing energy-storage related methods, articles and systems.

The disclosure relates to various compositions, related systems and articles, and methods of making and using the same. In some aspects, the disclosure relates to compositions containing a nanostructured organic compound, compositions containing an organic compound and a metal-organic framework embedded within the organic compound, and compositions containing an organic compound that is at least partially crystalline and a crystalline metal oxide distributed within the organic compound, as well as related methods of making, methods of use, articles (e.g., electrodes), and systems (e.g., energy storage systems, systems containing such energy storage systems) from the compositions of the disclosure. In some aspects, the disclosure relates to a composition that includes a silicon-containing material and a polymer made of imide monomers, as well as related systems and articles, and methods of making and using the same.

The compositions, articles and/or systems disclosed herein can exhibit one or more beneficial features. As an example, in some embodiments, an electrode containing a composition according to the disclosure can have a relatively high bulk electrical conductivity, metal ion storage capacity, coulombic efficiency, and/or metal ion diffusion rate relative to certain other energy storage materials, articles and/or systems. As an additional example, in some embodiments, an electrode containing a composition according to the disclosure can have a reduced resistance at the interface between the electrode and the electrolyte and/or a reduced Warburg coefficient relative to certain other energy storage materials, articles and/or systems. As another example, in certain embodiments, the compositions can be relatively inexpensive and/or have a reduced environmental impact compared to certain other energy storage materials, articles and/or systems. As a further example, in some embodiments, the technology according to the disclosure can be free of issues (e.g., electrode pulverization due to large volume changes causing failure) associated with electrochemical performance encountered with certain other battery anode materials, energy storage materials, articles and/or systems. As an additional example, in certain embodiments, electrodes made from the compositions can be used for hundreds of cycles without a substantial decrease in performance (e.g., little or no decrease in the maximum capacity). As another example, in some embodiments, electrodes and batteries containing a composition according to the disclosure can have a relatively high energy density while being safer relative to certain other electrodes and batteries. In some embodiments, the voltages at which the insertion and extraction of metal ions (such as lithium, sodium or potassium ions) into/out of the negative electrode containing the composition occurs are less than 1 V and sufficiently above 0 V with respect to the voltage associated with the metal/metal ion (such as Li⁺/Li, Na⁺/Na and K⁺/K, respectively). Without wishing to be bound by theory, this voltage characteristic prevents the metal from plating on the negative electrode of the energy storage devices, thereby increasing safety while providing the devices with relatively high energy density compared to certain other energy storage devices.

In some embodiments, the compositions can be employed in water treatment by reducing the concentration of organic contaminants by adsorption and/or photocatalytic degradation.

In some embodiments, the methods of the disclosure can allow the consumption (e.g., depolymerization) of waste plastics (e.g., polyethylene terephthalate (PET) from plastic water bottles) into monomers (e.g., terephthalic acid). In some embodiments, the depolymerization of waste plastic is performed without the use of potentially dangerous (e.g. acidic, alkaline) chemicals, high pressures, high temperatures, and/or prolonged treatment with catalysts and/or reducing agents. Rather, in some embodiments, the methods of the disclosure allow the depolymerization of waste plastics with relatively benign reagents (e.g., SnCl₂, ZnCl₂, LiCl, and/or KCl), relatively low temperatures, relatively low pressures (e.g., atmospheric pressure), relatively short processing times, and without the use of acids, bases and/or enzymes. Thus, in certain embodiments, the methods can allow the depolymerization of waste plastics in a relatively safe, simple, inexpensive and fast manner, while also providing improved scalability, relatively easy separations of monomers and related compounds without the need for additional separation purification steps, and/or reduced costs relative to certain other depolymerization methods. In certain embodiments, the methods of the disclosure can produce compositions with relatively easy (e.g., no) purification of the compositions. For example, in some embodiments, the methods of the disclosure can generate a composition containing a first organic compound, and a second organic compound that can be separated from the composition relatively easily (e.g., by evaporation). Thus, in some embodiments, the methods of the disclosure can allow the depolymerization of PET at atmospheric pressure into nanostructured terephthalic acid (TPA), without the presence of other monomers, such as ethylene glycol.

In certain embodiments, the methods can depolymerize plastics into nanostructured and/or nanocrystalline monomers and related compounds, which are morphologically and/or structurally different from certain other materials (e.g., commercially available terephthalic acid that is not nanostructured and/or nanocrystalline).

In some embodiments, the methods of the disclosure can allow the consumption (e.g., depolymerization) of waste plastics (e.g., polyethylene terephthalate (PET) from plastic water bottles) to generate the compositions of the disclosure. In some embodiments, the methods do not involve using environmentally problematic and/or expensive chemicals, which, in certain cases, are often used in the manufacture of battery anode materials. In contrast, in some embodiments, the methods of the disclosure can employ relatively inexpensive, abundant and safe regents with little or no carcinogenicity and/or genotoxicity (e.g., SnCl₂, LiCl, KCl). Without wishing to be bound by theory, it is believed that, in certain embodiments, the conversion of SnCl₂ to SnO₂ in certain methods of the disclosure can lead to the release of chlorine gas. Such generated chlorine gas can optionally be used in one or more beneficial ways, including industrial applications (e.g., drinking water treatment).

In some embodiments, the methods of the disclosure can produce compositions with a nanostructured organic compound (e.g., terephthalic acid). Without wishing to be bound by theory, it is believed that, in some embodiments, the compounds produced by the methods of the disclosure can have a dark (e.g., black) color in appearance due to unique light-matter interactions with the nanostructured organic compound (e.g., nanostructured terephthalic acid) that are not present in other (i.e., non-nanostructured) forms of the organic compound.

In some embodiments, the methods of the disclosure can be used to separate mixtures of plastics by the selective depolymerization of some of the plastics in the mixture of plastics. For example, such methods can depolymerize certain polymers that can be depolymerized by the methods of the disclosure (e.g., PET), while not depolymerizing non-depolymerizable plastics (e.g., high-density polyethylene (HDPE)) in the mixture, allowing relatively easy separation of the depolymerized polymer and the non-depolymerizable polymer.

In some embodiments, the compositions of the disclosure can demonstrate faster reaction kinetics relative to certain other organic compounds such as commercially available terephthalic acid that is not nanostructured and/or nanocrystalline. Thus, the compositions of the disclosure can be used to prepare materials such as disodium terephthalate (Na₂TP, Na₂C₈H₄O₄) and dilithium terephthalate (Li₂TP, Na₂C₈H₄O₄) as the electrodes of Na-ion and Li-ion batteries, respectively, polymers such as poly(butylene adipate-co-terephthalate) (PBAT) and PET, and/or photosensitizing nanoparticles faster relative to certain other sources of terephthalic acid.

In some embodiments, the methods of the disclosure can allow the depolymerization of waste PET at atmospheric pressure into pure nanostructured terephthalic acid (TPA), without the presence of other types of monomers, such as ethylene glycol.

In general, commercially available TPA is known to have various applications in diverse fields including the detection of hydroxyl radicals in solutions and the preparation of 2-hydroxy-terephthalic acid applicable in biomedical cancer and water treatments, preparation of metal organic frameworks and photosensitizing nanoparticles applicable in biomedical imaging, preparation of various polymers such as poly(butylene adipate-co-terephthalate) (PBAT) and PET, and the preparation of electrode materials for metal-ion batteries, for example disodium terephthalate (Na₂TP, Na₂C₈H₄O₄) and dilithium terephthalate (Li₂TP, Na₂C₈H₄O₄) as the electrodes of Na-ion and Li-ion batteries, respectively.

In some embodiments, the methods of the disclosure not only provide relatively simple, fast and sustainable methods of depolymerizing PET into TPA, but also provide methods of forming nanostructured TPA which is believed to differ from many commercially available forms of TPA due to, for example, unique morphological characteristics, which allows the material to be used in all applications considered for TPA and more, while enhancing the kinetics of those processes. Replacement of commercially available TPA with nanostructured TPA in various material production processes can result in relatively fast, simple and efficient reactions, which can yield economic and/or technical advantages. This can also contribute to the reduction of greenhouse emissions associated with primary production of TPA.

In certain embodiments, the methods can depolymerize plastics into nanostructured and/or nanocrystalline monomers and related compounds and composites with a range of desirable new and/or known applications. Examples of such applications can include: the preparation of electrode materials and/or electrolyte materials and/or other functional components and/or structural components for energy generation and energy storage devices; the preparation of electrode materials and/or electrolyte materials for energy storage devices; the preparation of agents applicable in water purification; as template for preparation of other nanostructured materials applicable in energy/environmental protection/biomedicine and/or structural materials; the preparation of composite materials with enhanced mechanical and/or physical properties relative to certain other alternative composites.

In some embodiments, the methods of the disclosure can provide relatively facile and fast depolymerization of PET into two or more types of monomers, among which one monomer (e.g., TPA) is a condensed compound during the process, while other monomers are in the gas phase and leave the reactor during the process. This can reduce, if not completely avoid, post-purification processes used to separate mixed monomers, commonly employed in current chemical depolymerization technologies. Hence, in some embodiments, the methods of the disclosure can reduce the cost and complexity of monomer synthesis.

In some embodiments, the disclosure provides a relatively low-cost and relatively available materials for relatively sustainable development of current and emerging technologies, such as green energy production/storage and composites.

In an aspect, the disclosure provides a composition, including: a nanostructured organic compound including a plurality of molecules having the formula CₓO_{y}H_{z}, wherein: x is from 2 to 12; y is from 2 to 8; and z is from 2 to 14.

In an aspect, the disclosure provides a composition including: an organic compound; and a metal-organic framework embedded within the organic compound.

In an aspect, the disclosure provides a composition, including: an organic compound; and a crystalline metal oxide, wherein the organic compound is at least partially crystalline, and the crystalline metal oxide is distributed within the organic compound.

In an aspect the disclosure provides a composition, including: a material including silicon; and a polymer made of imide monomers, wherein a portion of imide monomers are cross-linked, and a portion of the material and a portion of the polymer are hydrogen bonded with each other.

In an aspect, the disclosure provides a composition, including: a polymer; and an organic compound including a plurality of molecules having the formula CₓO_{y}H_{z}, wherein: the organic compound is crystalline; x is from 2 to 12; y is from 2 to 8; and z is from 2 to 14.

In some embodiments, the organic compound is nanocrystalline.

In some embodiments, the composition includes crystalline domain sizes of 1 nm to 100 nm.

In some embodiments, the composition includes crystalline domain sizes of 20 nm to 80 nm.

In some embodiments, the composition includes crystalline domain sizes of 30 nm to 70 nm.

In some embodiments, a component of the composition has at least one dimension below 100 nm.

In some embodiments, a component of the composition has at least one dimension below 50 nm.

In some embodiments, a component of the composition has at least one dimension below 10 nm.

In some embodiments, the nanostructured organic compound has at least one dimension below 2 nm.

In some embodiments, the composition further includes at least one member selected from a metal oxide, a metal, a metal-organic framework, a silicon-containing material, and a graphene-containing material embedded within the nanostructured organic compound.

In some embodiments, the composition further includes a crystalline metal oxide embedded within the nanostructured organic compound.

In some embodiments, the composition has X-Ray diffraction (XRD) peaks. In some embodiments, the 2-theta (±0.2 degrees) values for the XRD peaks of the composition include at least one member selected from 16.99, 24.83 and 27.54 degrees. In some embodiments, the 2-theta (±0.2 degrees) values for the XRD peaks of the composition include at least two members selected from 16.99, 24.83 and 27.54 degrees. In some embodiments, the 2-theta (±0.2 degrees) values for the XRD peaks of the composition include 16.99, 24.83 and 27.54 degrees. In some embodiments, the composition has an XRD pattern with characteristic peaks as substantially shown in FIG. 36d.

In some embodiments, the crystalline metal oxide is uniformly distributed within the organic compound.

In some embodiments: in an interior region of the composition, the composition has a first concentration of the crystalline metal oxide; at a surface region of the composition, the composition has a second concentration of the crystalline metal oxide; and/or the first concentration is greater than the second concentration. In some embodiments, the first concentration is from 1 wt. % to 95 wt. %. In some embodiments, the first concentration is from 5 wt. % to 80 wt. %. In some embodiments, the first concentration is from 10 wt. % to 70 wt. %. In some embodiments, the second concentration is from 0.1 wt. % to 80 wt. %. In some embodiments, the second concentration is from 1 wt. % to 70 wt. %. In some embodiments, the second concentration is from 5 wt. % to 60 wt. %.

In some embodiments, the organic compound and the crystalline metal oxide are bound via hydrogen bonding.

In some embodiments, the composition includes nanoparticles with sizes of 1 nm to 200 nm.

In some embodiments, the composition includes nanoparticles with sizes of 1 nm to 100 nm, such as 0.01 μm to 100 μm.

In some embodiments, the composition forms particles with a size of 10 μm to 100 μm, such as 1 nm to 200 nm.

In some embodiments, the particles include sheet-like particles with sizes of 1 nm to 1 μm, such as 10 nm to 500 nm.

In some embodiments, the organic compound includes an amorphous phase.

In some embodiments, the organic compound is at least partially crystalline.

In some embodiments, the organic compound is crystalline.

In some embodiments, the organic compound is nanostructured.

In some embodiments, the organic compound has the formula CₓO_{y}H_{z}. In some embodiments: x is from 2 to 12; y is from 2 to 8; and z is from 2 to 14. In some embodiments: x is 8; y is from 4 to 6; and z is 4.

In some embodiments, the organic compound includes terephthalic acid, terephthalate, dimethyl terephthalate, Bis(2-Hydroxyethyl) terephthalate, ethylene glycol, phthalic acid, protocatechuic acid, and/or isophthalic acid. In some embodiments, the organic compound includes terephthalic acid.

In some embodiments, the organic compound includes an anorthic crystal system.

In some embodiments, the composition includes from 1 weight percent (wt. %) to 99 wt. % of the organic compound, such as 5 wt. % to 95 wt. % of the organic compound, or 10 wt. % to 90 wt. % of the organic compound.

In some embodiments, the crystalline metal oxide includes crystalline metal oxide nanoparticles.

In some embodiments, the composition includes from 1 wt. % to 95 wt. % of the crystalline metal oxide, such as from 5 wt. % to 90 wt. % of the crystalline metal oxide, or from 10 wt. % to 85 wt. % of the crystalline metal oxide.

In some embodiments, the crystalline metal oxide has a particle size from 1 nm to 100 nm, such as from 1 nm to 50 nm, from 1 nm to 10 nm, or from 1 nm to 5 nm.

In some embodiments, the crystalline metal oxide includes tin(IV) oxide (SnO₂), tin(II) oxide (SnO), zinc oxide (ZnO), zinc peroxide (ZnO₂), a calcium oxide, a lithium oxide, a potassium oxide, a lead oxide and/or an iron oxide. In some embodiments, the crystalline metal oxide includes tin(IV) oxide (SnO₂).

In some embodiments, the crystalline metal oxide has a tetragonal crystal system.

In some embodiments, the organic compound has X-Ray diffraction (XRD) peaks, and the crystalline metal oxide has XRD peaks. In some embodiments, the 2-theta (±0.2 degrees) values for the XRD peaks of the organic compound include at least one member selected from 17.41°, 25.21°, and 27.95° degrees.

In some embodiments, the 2-theta (±0.2 degrees) values for the XRD peaks of the organic compound include at least two members selected from 17.41°, 25.21°, and 27.95° degrees. In some embodiments, the 2-theta (±0.2 degrees) values for the XRD peaks of the organic compound include 17.41°, 25.21°, and 27.95° degrees. In some embodiments, the 2-theta (±0.2 degrees) values for the XRD peaks of the crystalline metal oxide include at least one member selected from 26.60°, 33.90°, 37.97°, 39.00°, 51.81°, 54.79°, 57.87°, 61.92°, 64.79°, 66.01°, 71.33°, 78.76°, 81.19°, 83.78°, and 87.290 degrees. In some embodiments, the 2-theta (±0.2 degrees) values for the XRD peaks of the crystalline metal oxide include at least two members selected from 26.60°, 33.90°, 37.97°, 39.00°, 51.81°, 54.79°, 57.87°, 61.92°, 64.79°, 66.01°, 71.33°, 78.76°, 81.19°, 83.78°, and 87.29° degrees. In some embodiments, the 2-theta (±0.2 degrees) values for the XRD peaks of the crystalline metal oxide include at least three members selected from 26.60°, 33.90°, 37.97°, 39.00°, 51.81°, 54.79°, 57.87°, 61.92°, 64.79°, 66.01°, 71.33°, 78.76°, 81.19°, 83.78°, and 87.29° degrees. In some embodiments, the 2-theta (±0.2 degrees) values for the XRD peaks of the crystalline metal oxide include at least four members selected from 26.60°, 33.90°, 37.97°, 39.00°, 51.81°, 54.79°, 57.87°, 61.92°, 64.79°, 66.01°, 71.33°, 78.76°, 81.19°, 83.78°, and 87.29° degrees.

In some embodiments, the composition has an XRD pattern with characteristic peaks as substantially shown in FIG. 6c.

In some embodiments, according to a differential scanning calorimetry (DSC) thermogram, the composition has at least one endothermic peak with a maximum temperature (±3° C.) selected from 328° C., 461° C. or 528° C. In some embodiments, according to a DSC thermogram the composition has at least two endothermic peaks with a maximum temperature (±3° C.) selected from 328° C., 461° C. and 528° C. In some embodiments, according to a DSC thermogram, the composition has endothermic peaks with and a maximum temperature (±3° C.) including 328° C., 461° C. and 528° C.

In some embodiments, the composition has a DSC thermogram substantially as depicted in FIG. 11.

In some embodiments, the composition has a thermal gravimetric analysis (TGA) thermogram substantially as depicted in FIG. 11.

In some embodiments, the composition has an X-ray photoelectron spectroscopy (XPS) spectrum substantially as depicted in FIG. 15e.

In some embodiments, the composition has a surface area of from 10 square meters per gram (m² g⁻¹) to 50 m² g⁻¹, such as from 15 m² g⁻¹ to 30 m² g⁻¹ or from 17 m² g⁻¹ to 21 m² g⁻¹.

In some embodiments, the composition has a bulk electrical conductivity of from 5 Siemens per meter (S m⁻¹) to 5000 S m⁻¹ at 6.3 MPa, such as from 100 S m⁻¹ to 1500 S m¹ at 6.3 MPa, or 400 S m⁻¹ to 600 S m⁻¹ at 6.3 MPa.

In some embodiments, the composition further includes a silicon-containing material. In some embodiments, the silicon-containing material is embedded into the organic compound. In some embodiments, the composition includes from 0.1 wt. % to 95 wt. % of the silicon-containing material, such as from 5 wt. % to 90 wt. % of the silicon-containing material, or from 10 wt. % to 85 wt. % of the silicon-containing material. In some embodiments, the silicon-containing material includes elemental silicon. In some embodiments, the silicon-containing material includes nanoparticles. In some embodiments, the silicon-containing material nanoparticles have a particle size of from 1 nanometer (nm) to 1000 nm, such as from 1 nm to 500 nm, from 1 nm to 20 nm, or from 1 nm to 5 nm.

In some embodiments, the composition further includes graphene nanosheets. In some embodiments, the graphene nanosheets cover at least a portion of the crystalline metal oxide. In some embodiments, the composition includes from 0.1 wt. % to 50 wt. % of the graphene nanosheets, such as from 5 wt. % to 45 wt. % of the graphene nanosheets, or from 10 wt. % to 40 wt. % of the graphene nanosheets. In some embodiments, the graphene nanosheets include flakes having a flake size of from 1 nm to 5 μm, such as from 50 nm to 5 μm, or from 100 nm to 1 μm. In some embodiments, the graphene nanosheets include from 1 layer to 100 layers, such as from 1 layer to 50 layers, or from 1 layer to 10 layers. In some embodiments, the graphene nanosheets have a carbon purity of at least 90%, such as at least 95%. In some embodiments, the graphene nanosheets include functional groups on their surface. In some embodiments, the functional groups include a hydroxyl group, a carbonyl group, a carboxyl group and/or an amino group. In some embodiments, the composition further includes a silicon-containing material, and the graphene nanosheets cover at least a portion of the silicon-containing material.

In some embodiments, the composition further includes a metal-organic framework embedded within the organic compound.

In some embodiments, the metal-organic framework is at least partially crystalline.

In some embodiments, the metal-organic framework is crystalline.

In some embodiments, the metal-organic framework is nanostructured.

In some embodiments, the metal-organic framework includes the organic compound and a metal. In some embodiments, the metal is Zn, Fe, Cu, Al, Zr, Cr, Co, Li, Na or K. In some embodiments, the metal is Zn.

In some embodiments, the metal-organic framework has the formula MCₓH_{y}O_{z}·nH₂O, wherein M is a metal and n is 0 to 5 (e.g., 0, 1, 2 or 3.5). In some embodiments, the metal-organic framework has the formula MCₓH_{y}O_{z}·3.5H₂O wherein M is a metal. In some embodiments, the metal-organic framework has the formula MCₓH_{y}O_{z} wherein M is a metal. In some embodiments: x is from 2 to 12; y is from 2 to 8; and z is from 2 to 14. In some embodiments: x is 8; y is 4; and z is 4.

In some embodiments, the metal-organic framework includes terephthalate.

In some embodiments, the composition forms particles with a size of 500 nm to 700 μm. In some embodiments, the particles include sheet-like particles with sizes of 10 nm to 10 μm. In some embodiments, the sheet-like particles have a thickness of 1 nm to 10 nm. In some embodiments, the sheet-like particles include nanoparticles with sizes of 1 nm to 60 nm. In some embodiments, the particles include agglomerated nanoparticles with sizes of 1 nm to 60 nm. In some embodiments, the particles include metal-organic framework crystals with sizes of 10 nm to 10 μm.

In some embodiments, the metal-organic framework has an average crystalline domain size of 30 nm to 60 nm.

In some embodiments, the 2-theta (±0.2 degrees) values for the XRD peaks of the composition include at least one member selected from 11.75, 14.74, 16.60 and 16.98 degrees. In some embodiments, the 2-theta (±0.2 degrees) values for the XRD peaks of the composition include at least two members selected from 11.75, 14.74, 16.60 and 16.98 degrees. In some embodiments, the 2-theta (±0.2 degrees) values for the XRD peaks of the composition include 11.75, 14.74, 16.60 and 16.98 degrees.

In some embodiments, the composition has an XRD pattern with characteristic peaks as substantially shown in FIG. 60a.

In some embodiments, the 2-theta (±0.2 degrees) values for the XRD peaks of the composition include at least one member selected from 9.89, 19.33, 25.27, and 40.11 degrees. In some embodiments, the 2-theta (±0.2 degrees) values for the XRD peaks of the composition include at least two members selected from 9.89, 19.33, 25.27, and 40.11 degrees. In some embodiments, the 2-theta (±0.2 degrees) values for the XRD peaks of the composition include 9.89, 19.33, 25.27, and 40.11 degrees.

In some embodiments, the 2-theta (±0.2 degrees) values for the XRD peaks of the composition include at least one member selected from 17.45, 25.27, 28.017 and 42.99 degrees. In some embodiments, the 2-theta (±0.2 degrees) values for the XRD peaks of the composition include at least two members selected from 17.45, 25.27, 28.017 and 42.99 degrees. In some embodiments, the 2-theta (±0.2 degrees) values for the XRD peaks of the composition include 17.45, 25.27, 28.017 and 42.99 degrees. In some embodiments, the composition has an XRD pattern with characteristic peaks as substantially shown in FIG. 60b.

In some embodiments, the composition further includes a tin-containing member selected from metallic tin, tin chloride, tin chloride hydrate, tin chloride hydroxide and tin oxide chloride hydroxide (e.g., having the formula Sn₂₁Cl₁₆(OH)₁₄O₆). In some embodiments, the tin-containing member includes particles with sizes of 1 nm to 100 nm. In some embodiments, the tin-containing member is at least partially crystalline. In some embodiments, the tin-containing member is crystalline. In some embodiments, the tin-containing member is nanostructured.

In some embodiments, the composition is a dark powder.

In some embodiments, the composition is a black powder.

In some embodiments, the composition has an absorbance of at least 1 a.u. (e.g., at least 1.5 a.u.) at 242 nm at a concentration of 0.5 g/L.

In some embodiments, the composition has an absorbance of at least 0.4 a.u. at 450 nm at a concentration of 0.5 g/L.

In some embodiments, the composition has an absorbance of at least 0.4 a.u. at 500 nm at a concentration of 0.5 g/L.

In some embodiments, the composition has an absorbance of at least 0.5 a.u. at 317 nm at a concentration of 0.5 g/L.

In some embodiments, the composition further includes a metal embedded within the organic compound. In some embodiments, the metal is selected from Na, Li, K and Zn. In some embodiments, the composition includes the molecular formula selected from Na₂C₈H₄O₄, Li₂C₈H₄O₄, K₂C₈H₄O₄, or ZnC₈H₆O₄.

In some embodiments, the composition consists of nanostructured organic compound.

In some embodiments, the composition consists of nanostructured terephthalic acid.

In as aspect, the disclosure provides an electrode that includes a composition according to the disclosure. In some embodiments, the composition further includes at least one member selected from conductive carbon, a binder copper foil, and graphene nanosheets. In some embodiments, the binder includes polyimide.

In some embodiments, the electrode has a Li-ion discharge capacity of from 10 milli-Ampere hours per gram (mAh g⁻¹) to 1500 mAh g⁻¹ (e.g., from 100 mAh g⁻¹ to 1000 mAh g⁻¹, from 200 mAh g⁻¹ to 800 mAh g⁻¹) after 500 cycles at a current density of 200 mA g⁻¹.

In some embodiments, the electrode has a coulombic efficiency of from 70% to 120% (e.g., 80% to 110%, 90% to 100%) after 500 cycles.

In some embodiments, a half-cell made of the electrode has an increase in electrolyte resistance of from 1.0Ω to 6.0Ω (e.g., from 2.0Ω to 5.0Ω, from 3.0Ω to 4.0Ω) after 150 cycles.

In some embodiments, a half-cell made of the electrode has an increase in electrolyte resistance of from 2.0Ω to 8.0Ω (e.g., 2.0Ω to 7.0Ω, from 3.0Ω to 6.0Ω) after 300 cycles.

In some embodiments, the electrode has a lithium ion diffusion rate of from 10⁻¹¹ cm²s⁻¹ to 9×10⁻⁹ cm²s⁻¹ (e.g., from 5×10⁻¹¹ cm²s⁻¹ to 5×10⁻⁹ cm²s⁻¹, from 9×10⁻¹¹ cm²s⁻¹ to 10⁻⁹ cm²s⁻¹) after 150 cycles. In some embodiments, the electrode has a lithium ion diffusion rate of from 2×10⁻¹¹ cm²s⁻¹ to 10⁻⁸ cm²s⁻¹ (e.g., from 7×10⁻¹¹ cm²s⁻¹ to 7×10⁻⁹ cm²s⁻¹, from 10⁻¹⁰ cm²s⁻¹ to 9×10⁻⁹ cm²s⁻¹) after 300 cycles.

In some embodiments, the electrode has a Li-ion discharge capacity of from 300 mAh g⁻¹ to 1500 mAh g⁻¹ after 10 cycles at a current density of 100 mA g⁻¹. In some embodiments, the electrode has a Li-ion discharge capacity of from 200 mAh g⁻¹ to 1500 mAh g⁻¹ after 30 cycles at a current density of 500 mA g⁻¹. In some embodiments, the electrode has a Li-ion discharge capacity of from 200 mAh g⁻¹ to 1500 mAh g⁻¹ after 50 cycles at a current density of 1000 mA g⁻¹. In some embodiments, the electrode has a Li-ion discharge capacity of from 50 mAh g⁻¹ to 1300 mAh g⁻¹ after 60 cycles at a current density of 5000 mA g⁻¹.

In some embodiments, a Na-ion insertion into of the electrode occurs at a voltage of 0.1 V to 0.9 V versus Na/Na⁺. In some embodiments, a Na-ion insertion into of the electrode occurs at a voltage of 0.15 V to 0.8 V versus Na/Na⁺. In some embodiments, a Na-ion insertion into of the electrode occurs at a voltage of 0.17 V to 0.3 V versus Na/Na⁺.

In some embodiments, a Na-ion extraction out of the electrode occurs at a voltage of 0.3 V to 0.7 V versus Na/Na⁺. In some embodiments, a Na-ion extraction out of the electrode occurs at a voltage of 0.4 V to 0.6 V versus Na/Na⁺.

In some embodiments, the electrode has a Li-ion discharge capacity of from 100 mAh g⁻¹ to 1800 mAh per g⁻¹ after 500 cycles.

In some embodiments, the electrode has a Li-ion discharge capacity of from 100 milliAmpere hours (mAh) per gram of the crystalline metal oxide to 1800 mAh per gram of the crystalline metal oxide after 500 cycles. In some embodiments, the electrode has a Li-ion discharge capacity of from 200 mAh per gram of crystalline metal oxide to 1800 mAh per gram of crystalline metal oxide after 500 cycles. In some embodiments, the electrode has a Li-ion discharge capacity of from 500 mAh per gram of the crystalline metal oxide to 1800 mAh per gram of the crystalline metal oxide after 500 cycles.

In aspect, the disclosure provides a battery including an electrode according to the disclosure. In some embodiments, the electrode is an anode. In some embodiments, the battery further includes a cathode. In some embodiments, the battery further includes an electrolyte between the anode and the cathode. In some embodiments, there is an electrical connection between the anode and the cathode. In some embodiments, the battery is a lithium-ion battery, a sodium-ion battery, a calcium-ion battery or a potassium-ion battery.

In an aspect, the disclosure provides a car including a battery according to the disclosure.

In an aspect, the disclosure provides a structure including a battery according to the disclosure.

In an aspect, the disclosure provides a system capable of generating electricity, wherein the system is configured to be in electrical contact with a battery according to the disclosure. In some embodiments, the system includes a system capable of generating electricity from electromagnetic radiation, a system capable of generating electricity from rotation of a turbine, and/or a system capable of generating electricity from a combustion reaction. In some embodiments, the system includes at least one member selected from a photovoltaic, a wind turbine, a hydroelectric turbine, a hydroelectric power station, a nuclear power station, a coal-fired power station, an oil power plant, and a gas power plant. In some embodiments, the battery is configured to store electricity generated by the system.

In some embodiments, the composition includes a transition metal dichalcogenide. In some embodiments, the transition metal dichalcogenide has a formula MX₂. In some embodiments, M is a transition metal atom, and X is a chalcogen atom. In some embodiments, M includes Mo or W. In some embodiments, the chalcogen atom includes a member selected from the group consisting of S, Se and Te. In some embodiments, the transition metal dichalcogenide includes a two dimensional transition metal dichalcogenide. In some embodiments, the transition metal dichalcogenide is embedded within the organic compound. In some embodiments, the composition includes a crystalline metal oxide, and the transition metal dichalcogenide is embedded within the crystalline metal oxide.

In some embodiments, the composition includes a polymer and a material including silicon. In some embodiments, the polymer is made of monomers, and at least some of the monomers are cross-linked with each other. In some embodiments, the monomers include imide monomers. In some embodiments, a portion of the material and a portion of the polymer are hydrogen bonded with each other. In some embodiments, the material includes silicon particles embedded within the organic compound. In some embodiments, the material includes silicon particles. In some embodiments, the silicon particles have sizes of from 1 nm to 5 μm, optionally from 10 nm to 1 μm, optionally from 20 nm to 500 nm, optionally from 20 nm to 200 nm. In some embodiments, the crystalline organic compound is nanostructured and nanocrystalline. In some embodiments, a Li-ion storage charge capacity of composition at a current density of 200 mA/g is from 1000 mAh/g and to 3500 mAh/g after 30 cycles, based on a mass of silicon element in the material. In some embodiments, a Li-ion storage charge process of the composition at a current density of 200 mA/g provides a specific energy density of from 3000 Wh kg⁻¹ to 8000 Wh kg⁻¹ at the 30th cycle. In some embodiments, the composition has a Li-ion diffusion impedance from 10Ω to 60Ω, optionally from 20Ω to 40Ω. In some embodiments, an infrared spectrum of the polymer has a peak at 1723 cm⁻¹ and/or a peak at 1362 cm⁻¹. In some embodiments, an infrared spectrum of the composition is devoid of a peak at 3739 cm⁻¹. In some embodiments, the polymer is a polyimide.

In an aspect, the disclosure provides a method including: disposing a composition according to the disclosure into a first solution composing a contaminant at a first concentration; and reducing the concentration of the contaminant in the first solution to form a second solution, wherein a concentration of the contaminant in the second solution is less than a concentration of the contaminant in the first solution. In some embodiments, at least a portion of the contaminant in the first solution is adsorbed onto a surface of the composition. In some embodiments, the composition photocatalytically degrades at least a portion of the contaminant in the first solution.

In some embodiments, reducing the concentration of the contaminant in the first solution further includes exposing the first solution to visible light. In some embodiments, the contaminant includes a member selected from a hydrocarbon, an azo dye and a xanthate-based compound.

In an aspect, the disclosure provides a method including: heating a mixture including a first polymer, a second polymer and a depolymerization agent to a first temperature; depolymerizing the first polymer to form an organic compound; and forming a composition including the organic compound, wherein the second polymer is not substantially depolymerized. In some embodiments, the first polymer is a polymer that can be depolymerized in the presence of water. In some embodiments, the first polymer includes polyethylene terephthalate (PET), polystyrene, polyvinyl chloride, nylon, a polyurethane, a phenolic resin and/or an epoxy resin. In some embodiments, second polymer includes a polyethylene and/or a polypropylene. In some embodiments, method further includes separating the composition from the second polymer.

In an aspect, the disclosure provides a method, including: heating a mixture including reactants to a first temperature; and forming a composition including a nanocrystalline organic compound, wherein the reactants include a polymer and a depolymerization agent. In some embodiments, the reactants further include a salt and heating the mixture makes the salt a molten salt.

In an aspect, the disclosure provides a method, including: heating a mixture including reactants to a first temperature; and forming a composition including an organic compound and a crystalline metal oxide, wherein: the organic compound is at least partially crystalline; the reactants include a salt, a polymer and a depolymerization agent; and heating the salt makes the salt a molten salt.

In an aspect, the disclosure provides a method of making a composition according to the disclosure, wherein the method includes heating a mixture including the polymer to a temperature of from 150° C. to 400° C. In some embodiments, the method includes heating for a time of from 1 second to 5 hours. In some embodiments, heating is conducted in an inert atmosphere or a nitrogen atmosphere. In some embodiments, the atmosphere includes hydrogen in a range of from 0.1% to 99.9%.

In some embodiments, heating the mixture depolymerizes the polymer to provide monomers, and the monomers form the organic compound. the reactants include from 1 weight percent (wt. %) to 99 wt. % of the polymer.

In some embodiments, the reactants include from 5 wt. % to 95 wt. % (e.g., from wt. % to 90 wt. %) of the polymer.

In some embodiments, the polymer includes polyethylene terephthalate (PET), poly(acrylonitrile), poly(6-aminocaproic acid), poly(caprolactam), Nylon, poly(etheretherketone) (PEEK), Poly(ethylene) (PE), poly(hexamethylene adipamide), poly(methyl methacrylate), poly(methylene oxide), poly(4-methylpentene), poly(propylene), poly(styrene), poly(trans-1,4-butadiene), poly(vinyl alcohol), poly(vinyl chloride) (PVC), poly(vinyl fluoride), poly(vinylidene chloride), and/or poly(vinylidene fluoride).

In some embodiments, the polymer includes polyethylene terephthalate (PET).

In some embodiments, the polymer is derived from a waste plastic.

In some embodiments, a difference in melting temperatures of the polymer and the depolymerization agent is less than 100° C. (e.g., less than 75° C., less than 50° C.).

In some embodiments, the reactants include from 1 wt. % to 95 wt. % (e.g., from 5 wt. % to 90 wt. %, from 10 wt. % to 85 wt. %) of the depolymerization agent.

In some embodiments, the depolymerization agent includes an inorganic salt. In some embodiments, the inorganic salt includes a metal of the crystalline metal oxide. In some embodiments, the inorganic salt undergoes an oxidation during the heating.

In some embodiments, the depolymerization agent includes tin(II) chloride (SnCl₂), zinc chloride (ZnCl₂), calcium chloride (CaCl₂)), lead chloride (PbCl₂), sodium chloride (NaCl), potassium chloride (KCl) and/or iron chloride (FeCl₂). In some embodiments, the depolymerization agent includes tin(II) chloride (SnCl₂). In some embodiments, the depolymerization agent includes zinc chloride (ZnCl₂).

In some embodiments, the depolymerization agent includes an ionic liquid. In some embodiments, the ionic liquid includes of [bmpy][Tf₂N] and [BMIM][Tf₂N] and/or Imidazolium ionic liquids. In some embodiments, the ionic liquid includes a cation selected from 1-octyl-3-methyl-imidazolium ([OMIM]), 1-methyl-imidazolium ([MIM]), 1-ethyl-3-methyl-imidazolium ([EMIM]), 1,3-dimethyl-imidazolium ([M13IM]), 1-(2-hydroxylethyl)-3-methylimidazolium ([HOEMIm]), 1-ethyl-2,3-dimethyl-imidazolium ([EMMIM]), 1-butyl-3-methyl-imidazolium ([BMIM]), 1-hexyl-3-methyl-imidazolium ([HMIM]), 1,2,3-trimethyl-imidazolium ([MMMIM]), 1-decyl-3-methyl-imidazolium ([DMIM]), 1-allyl-3-butyl-imidazolium ([ABIM]), 1,2-dimethyl-imidazolium ([M₁₂IM]), 1-butyl-2,3-dimethyl-imidazolium ([BMMIM]), 1-allyl-3-methyl-imidazolium ([AMIM]), 1-allyl-3-vinyl-imidazolium ([AVIM]), tetradecyltrihexylphosphonium ([P66614]), N-ethyl-pyridinium ([EPy]), and N-butyl-pyridinium ([BPy]). In some embodiments, the ionic liquid includes an anion selected from bis(trifluoromethylsulfonyl)imide ([Tf₂N]), bromide ([Br]), dicyanamide ([DCA]), hexafluorophosphate ([PF₆]), perchlorate ([CO₄]), tosylate ([TS]), acetate ([Ac]), chloride ([Cl]), glycinate ([Gly]), iodide ([I]), trifluoromethanesulfonate ([TFO]), prolinate ([Pro]), alaninate ([Ala]), lysinate ([Lys]), dihydrogen phosphate ([H₂PO₄]), nitrate ([NO₃]), serinate ([Ser]), glutamate ([Glu]), and hydrogen sulfate ([HsO₄]), and tetrafluoroborate ([BF₄]).

In some embodiments, the salt includes a chloride salt. In some embodiments, the salt includes LiCl and/or KCl. In some embodiments, the salt includes LiCl and KCl. In some embodiments, the salt includes from 40 wt. % LiCl to 80 wt. % (e.g., 50 wt. % LiCl to 70 wt. %, 55 wt. % LiCl to 65 wt. %) LiCl. In some embodiments, the salt includes from 20 wt. % KCl to 70 wt. % (e.g., 30 wt. % KCl to 55 wt. %, 35 wt. % KCl to 40 wt. %) KCl.

In some embodiments, the salt has a melting point of from 250° C. to 700° C. (e.g., from 300° C. to 600° C., from 320° C. to 500° C.).

In some embodiments, heating is performed under an atmosphere selected from air, nitrogen gas, argon, and hydrogen gas.

In some embodiments, heating is performed under an inert atmosphere.

In some embodiments, heating is performed under argon.

In some embodiments, heating is performed under argon and hydrogen. In some embodiments, heating is performed under argon and from 1% to 99% hydrogen.

In some embodiments, heating is performed at a temperature greater than a melting point of the polymer and a melting point of the depolymerization agent.

In some embodiments, heating is performed at a temperature greater than a melting point of the polymer, a melting point of the salt and a melting point of the depolymerization agent.

In some embodiments, heating is performed at a temperature lower than a carbonization temperature of the polymer and a decomposition temperature of the organic compound.

In some embodiments, the first temperature is 200° C. to 600° C.

In some embodiments, the first temperature is at most 600° C.

In some embodiments, the first temperature is 200° C. to 450° C.

In some embodiments, the first temperature is at least 250° C.

In some embodiments, first temperature is at least 300° C.

In some embodiments, first temperature is at most 500° C.

In some embodiments, the first temperature is at most 400° C.

In some embodiments, the first temperature is at most 310° C.

In some embodiments, the mixture is held at the first temperature for 0.01 minute to 120 minutes (e.g., for 1 minute to 60 minutes, for 5 minutes to 30 minutes, 1 second).

In some embodiments, the mixture is heated at a rate of from 1° C. min⁻¹ to 100° C. min⁻¹ (e.g., from 2° C. min⁻¹ to 50° C. min⁻¹, 3° C. min⁻¹ to 20° C. min⁻¹) In some embodiments, the method further includes contacting the composition with a solvent. In some embodiments, the solvent includes at least one member selected from an aqueous solution, an alkali aqueous solution, an acidic aqueous solution and a polar organic liquid. In some embodiments, the solvent has a pH of from 0.1 to 7 (e.g., from 1 to 6, from 2 to 5). In some embodiments, the solvent includes an acid selected from hydrochloric acid, sulfuric acid, nitric acid and phosphoric acid. In some embodiments, the acid in the solvent is from 1 percent by volume (vol. %) to 98 vol. % (e.g., from 2 vol. % to 95 vol. %, from 5 vol. % to 90 vol. %). In some embodiments, the solvent has a pH of from 7 to 14 (e.g., from 8 to 13, from 9 to 12). In some embodiments, the solvent includes a hydroxide. In some embodiments, the hydroxide is selected from sodium hydroxide and potassium hydroxide. In some embodiments, the polar organic liquid includes ethanol.

In some embodiments, the composition includes the depolymerization agent and contacting the composition with the solvent removes at least a portion of the depolymerization agent. In some embodiments, the composition includes depolymerization agent and contacting the composition with the solvent hydrolyzes at least a portion of the depolymerization agent.

In some embodiments: the depolymerization agent includes SnCl₂; the hydrolysis of SnCl₂ forms a second material including tin, chlorine, hydrogen and oxygen; and the contacting forms a product including the composition and the second material dispersed within the organic compound of the composition. In some embodiments, the second material includes a tin oxide chloride hydroxide. In some embodiments, the second material includes a stoichiometry of Sn₂₁Cl₁₆(OH)₁₄O₆. In some embodiments, the second material is at least partially crystalline.

In some embodiments, the method further includes separating the composition from the solvent. In some embodiments, separation includes a method selected from vacuum filtration and centrifugation. In some embodiments, the method further includes, after separating the composition, drying the composition. In some embodiments, the composition is dried in an atmosphere selected from air, an inert atmosphere and a vacuum. In some embodiments, the composition is dried at a temperature of from −196° C. to 100° C. (e.g., from −196° C. to 0° C., from 20° C. to 100° C.).

In some embodiments, the solvent includes a precursor of a silicon-containing material, and contacting the composition with the solvent introduces the silicon-containing material into the composition.

In some embodiments, the mixture further includes a precursor of a silicon-containing material, and the composition further includes the silicon-containing material.

In some embodiments, the reactants include from 0.1 wt. % to 98 wt. % (e.g., 5 wt. % to 90 wt. %, 10 wt. % to 85 wt. %) of the precursor of the silicon-containing material.

In some embodiments, the precursor of the silicon-containing material includes nanoparticles.

In some embodiments, the precursor of the silicon-containing material includes elemental silicon, Ca₂Si, Ca₅Si₃, CaSi, Ca₃Si₄, CaSi₂ and/or Mg₂Si.

In some embodiments, the precursor of the silicon-containing material has a particle size of from 1 nm to 1000 nm (e.g., from 1 nm to 500 nm, from 1 nm to 20 nm, from 1 nm to 5 nm).

In some embodiments, the precursor of the silicon-containing material includes a surface-functionalization.

In some embodiments, the method further includes ball-milling the precursor of the silicon-containing material. In some embodiments, the ball-milling is performed in the presence of a solvent. In some embodiments, the solvent includes n-hexane. In some embodiments, the solvent includes graphene nanosheets, and contacting the composition with the solvent introduces the graphene nanosheets into the composition.

In some embodiments, the mixture further includes graphene nanosheets, and the composition further includes the graphene nanosheets. In some embodiments, the mixture includes from 0.1 wt. % to 80 wt. % (e.g., 2 wt. % to 45 wt. %, 5 wt. % to 40 wt. %) of the graphene nanosheets. In some embodiments, the graphene nanosheets include flakes having a flake size of from 1 nm to 5 μm (e.g., from 50 nm to 5 μm, from 100 nm to 1 μm). In some embodiments, the graphene nanosheets include from 1 layer to 100 layers (e.g., from 1 layer to 50 layers, from 1 layer to 10 layers). In some embodiments, the graphene nanosheets have a carbon purity of at least 90% (e.g., at least 95%). In some embodiments, a surface of the graphene nanosheets includes functional groups. In some embodiments, the functional groups include hydroxyl, carbonyl, carboxyl and amino groups. In some embodiments, the graphene nanosheets are produced through a cathodic electrochemical exfoliation of graphite electrodes. In some embodiments, the cathodic electrochemical exfoliation is performed in a molten salt. In some embodiments, the molten salt includes at least one member selected from lithium chloride and sodium chloride. In some embodiments, the cathodic electrochemical exfoliation is performed at a temperature of from 500° C. to 900° C.

In some embodiments, the organic compound includes an amorphous phase.

In some embodiments, the organic compound is crystalline.

In some embodiments, the organic compound is nanostructured. In some embodiments, the organic compound has the formula CₓO_{y}H_{z}. In some embodiments: x is from 2 to 12; y is from 2 to 8; and z is from 2 to 14. In some embodiments: x is 8; y is from 4 to 6; and z is 4.

In some embodiments, the organic compound includes terephthalic acid, terephthalate, dimethyl terephthalate, Bis(2-Hydroxyethyl) terephthalate, ethylene glycol, phthalic acid, protocatechuic acid, and/or isophthalic acid. In some embodiments, the organic compound includes terephthalic acid. In some embodiments, the terephthalic acid is nanostructured with at least one dimension below 100 nm.

In some embodiments, heating is performed at a pressure in the range from 0.01 to 100 atm (e.g., from 0.1 to 10 atm, from 0.5 to 5 atm, from 0.8 to 1.5 atm).

In some embodiments, heating is performed at atmospheric pressure.

In some embodiments, the method further includes: forming a suspension including the composition and graphene nanosheets; sonicating the suspension; and collecting a product from the suspension, wherein the product includes the composition and graphene nanosheets. In some embodiments, the suspension further includes an acid. In some embodiments, the product includes from 50 wt. % to 99.9 wt. % of the composition. In some embodiments, the product includes from 0.1 wt. % to 50 wt. % of the graphene nanosheets.

In some embodiments, the reactants further include water.

In some embodiments, at least a portion of the depolymerization agent is hydrated.

In some embodiments, the depolymerization agent is hydrated.

In some embodiments, the depolymerization agent includes from 0.1 wt. % to 20 wt. % water.

In some embodiments, the method further includes: removing at least a portion of the composition; and adding additional polymer.

In some embodiments, the composition further includes a metal-organic framework embedded within the organic compound. In some embodiments, the metal-organic framework includes the organic compound and a metal.

In some embodiments, the metal is selected from Zn, Fe, Cu, Al, Zr, Cr, Co, Li, Na and K. In some embodiments, the metal is Zn.

In some embodiments, the metal-organic framework has the formula MCₓH_{y}O_{z}·nH₂O, wherein M is a metal and n is 0 to 5 (e.g., 0, 1, 2 or 3.5).

In some embodiments, the metal-organic framework has the formula MCₓH_{y}O_{z}·3.5H₂O wherein M is a metal.

In some embodiments, the metal-organic framework has the formula MCₓH_{y}O_{z} wherein M is a metal. In some embodiments: x is from 2 to 12; y is from 2 to 8; and z is from 2 to 14. In some embodiments: x is 8; y is 4; and z is 4.

In some embodiments, the metal-organic framework includes terephthalate.

In some embodiments: the metal-organic framework includes hydrated water; and the method further includes: heating the composition to a second temperature, wherein the heating removes at least a portion of the hydrated water from the metal-organic framework. In some embodiments, the metal-organic framework includes a first crystal structure prior to the heating to the second temperature and the metal-organic framework includes a second crystal structure after the heating to the second temperature, wherein the second crystal structure is different from the first crystal structure. In some embodiments, the second temperature is 50° C. to 400° C. In some embodiments, the composition is held at the second temperature for 1 millisecond to 10 hours. In some embodiments, the method further includes cooling the mixture after heating the mixture. In some embodiments, cooling is performed under the same atmosphere as the heating.

In some embodiments, the reactants are free from acids, bases and enzymes.

In some embodiments, the method does not include a separation step.

In some embodiments, heating forms a second organic compound, and the second organic compound is evaporated.

In some embodiments, the second organic compound is ethylene glycol.

In some embodiments, the composition consists of nanostructured organic compound.

In some embodiments, the composition consists of nanostructured terephthalic acid.

In some embodiments, the method further includes reacting the composition with a metal hydroxide to form a product including the composition and a metal of the hydroxide, wherein the reacting is performed for at most 18 hours. In some embodiments, the product includes the molecular formula Na₂C₈H₄O₄, Li₂C₈H₄O₄, K₂C₈H₄O₄, or ZnC₈H₆O₄.

In some embodiments, the reacting is performed for at most 12 hours (e.g., at most 6 hours).

In some embodiments, the hydroxide is from selected from NaOH, LiOH, KOH and Zn(OH)₂.

In some embodiments, the reactants and the composition include a transition metal dichalcogenide. In some embodiments, the transition metal dichalcogenide has a formula MX₂, wherein M is a transition metal atom, and X is a chalcogen atom. In some embodiments, M includes Mo or W. In some embodiments, the chalcogen atom includes a member selected from the group consisting of S, Se and Te. In some embodiments, the transition metal dichalcogenide includes a two dimensional transition metal dichalcogenide. In some embodiments, the transition metal dichalcogenide is embedded within the organic compound. In some embodiments: the composition includes a crystalline metal oxide; and the transition metal dichalcogenide is embedded within the crystalline metal oxide.

### BRIEF DESCRIPTION OF THE FIGURES

FIG. 1 schematically illustrates an embodiment of a composition.

FIG. 2 schematically illustrates an embodiment of a composition.

FIG. 3 schematically depicts an embodiment of an electrode.

FIG. 4 schematically depicts an embodiment of a cell of a battery.

FIG. 5a is a reaction scheme.

FIG. 5b is a reaction scheme.

FIG. 5c is a reaction scheme.

FIG. 5d is a reaction scheme.

FIGS. 6a-f show graphs of X-ray diffraction (XRD) data.

FIGS. 7a-b show graphs of X-ray diffraction data.

FIGS. 8a-c show graphs of X-ray diffraction data.

FIG. 9 shows a graph of Raman spectroscopy data.

FIG. 10 shows a graph of Fourier transform infrared spectroscopy (FTIR) data.

FIG. 11 show a graph of differential scanning calorimetry (DSC) and thermal gravimetry analysis (TGA) data.

FIGS. 12a-d show scanning electron microscopy (SEM) micrographs.

FIGS. 13a-b show energy dispersive spectroscopy (EDS) mappings.

FIGS. 14a-d show transmission electron microscopy (TEM) data.

FIGS. 15a-h show graphs of X-ray photoelectron spectroscopy data.

FIG. 16 shows a bar graph electrical conductivity data.

FIGS. 17a-g show graphs of electrochemical characterization data.

FIG. 18 shows a graph of electrochemical characterization data.

FIGS. 19a-b show scanning electron microscopy micrographs.

FIGS. 20a-d show graphs of electrochemical characterization data.

FIG. 21 shows a graph of X-ray diffraction data.

FIG. 22 shows a transmission electron microscopy micrograph.

FIG. 23 shows a scanning electron microscopy micrograph.

FIG. 24 shows a scanning electron microscopy micrograph.

FIG. 25 shows a graph of X-ray diffraction data.

FIGS. 26a-b show scanning electron microscopy micrographs.

FIGS. 27a-d show scanning electron microscopy micrographs.

FIG. 28a shows a photograph of heat treated PET+(SnCl₂−LiCl−KCl).

FIG. 28b shows a photograph of commercial terephthalic acid.

FIG. 29a shows a photograph of waste PET.

FIG. 29b shows a photograph of shredded PET.

FIG. 29c shows a photograph a mixture of PET+SnCl₂+LiCl+KCl before heat treatment.

FIG. 29d shows a photograph a mixture of PET+SnCl₂+LiCl+KCl after heat treatment.

FIGS. 29e-f show photographs of a solidified salt disc.

FIG. 30 shows a graph of X-ray diffraction data.

FIG. 31 shows a graph of X-ray diffraction data.

FIGS. 32a-c show transmission electron microscopy micrographs.

FIGS. 33a-b show graphs of dye concentrations over time.

FIG. 34 show a graph of electrochemical characterization data.

FIG. 35a is a schematic of an experimental setup.

FIGS. 35b-c show temperature profiles as a function of time during heating.

FIGS. 36a-d show graphs of X-ray diffraction data.

FIGS. 37a-f show graphs of differential scanning calorimetry data.

FIGS. 38a-c show scanning electron microscopy micrographs.

FIGS. 39a-b show scanning electron microscopy micrographs.

FIGS. 40a-b show graphs of electrochemical characterization data.

FIG. 41 shows a graph of X-ray diffraction data.

FIG. 42 shows a scanning electron microscopy micrograph.

FIG. 43 shows a scanning electron microscopy micrograph.

FIG. 44 shows a graph of X-ray diffraction data.

FIG. 45 shows a graph of electrochemical characterization data.

FIG. 46 shows a graph of X-ray diffraction data.

FIG. 47 shows a graph of thermal gravimetry analysis data.

FIG. 48 shows a graph of differential scanning calorimetry data.

FIG. 49 shows a graph of X-ray diffraction data.

FIG. 50 shows a backscattered electron micrograph.

FIGS. 51a-c show energy dispersive X-ray spectra.

FIG. 52 shows energy dispersive spectroscopy mappings.

FIG. 53a shows a backscattered electron micrograph.

FIG. 53b shows a histogram of crystal sizes.

FIG. 54a-d show backscattered electron micrographs.

FIGS. 55a-d show secondary electron scanning electron micrographs.

FIG. 56 shows a transmission electron microscopy micrograph.

FIG. 57a shows a transmission electron microscopy micrograph.

FIG. 57b shows a histogram of particle size distribution.

FIG. 58a shows a transmission electron microscopy micrograph.

FIG. 58b shows a histogram of particle size distribution.

FIG. 59 shows a graph of electrochemical characterization data.

FIGS. 60a-b show graphs of X-ray diffraction data.

FIG. 61a-d show graphs of electrochemical characterization data.

FIG. 62 shows a graph of electrochemical characterization data.

FIG. 63 shows a graph of electrochemical characterization data.

FIG. 64a-b show photographs of HDPE before and after heat treatment, respectively.

FIGS. 65a-b show graphs of X-ray diffraction data.

FIGS. 66a-b show graphs of UV-Vis data.

FIGS. 67a-g show graphs of X-ray diffraction data.

FIG. 68 shows a schematic for a method of making electrodes.

FIGS. 69a-c show photographs of polyimide (PI).

FIG. 70 shows a graph of X-ray diffraction data.

FIG. 71 shows a schematic of a charge transfer complex among PI chains.

FIGS. 72a-d show graphs of FTIR data.

FIG. 73 shows a graph of UV-Vis data.

FIGS. 74a-b show graphs of FTIR data.

FIG. 74c shows a schematic of hydrogen bonding between a Si nanoparticle (“SiNP”) and PI.

FIGS. 75a-b show graphs of electrochemical characterization data.

FIG. 76 shows a graph of electrochemical characterization data.

FIGS. 77a-b show graphs of electrochemical characterization data.

FIGS. 78a-d show SEM micrographs.

FIG. 79a-e show graphs of X-ray diffraction data.

FIG. 80 shows a graph of electrochemical characterization data.

FIG. 81 shows cycling performance of an electrode.

### DETAILED DESCRIPTION

### Compositions

In some embodiments, a composition according to the disclosure can include a nanostructured organic compound including a plurality of molecules. A composition is said to be nanostructured if it includes one or more constituent parts that has a nanoscale size (e.g., 1 nm to 100 nm). In some embodiments, a component of the composition (e.g., the nanostructured organic compound) includes at least one dimension below 100 (e.g. below 95 below 90, below 85, below 80, below 75, below 70, below 65, below 60, below 55, below 50, below 45, below 40, below 35, below 30, below 25, below 20, below 15, below 10, below 5, below 4, below 3, below 2, below 1) nm. In general, the organic compound contains carbon, oxygen, and hydrogen (see discussion below). Generally, the organic compound has the formula CₓO_{y}H_{z}. In some embodiments, x is 2 to 12 (e.g., 2 to 3, 2 to 4, 2 to 5, 2 to 6, 2 to 7, 2 to 8, 2 to 9, 2 to 10, 2 to 11, 3 to 4, 3 to 5, 3 to 6, 3 to 7, 3 to 8, 3 to 9, 3 to 10, 3 to 11, 3 to 12, 4 to 5, 4 to 6, 4 to 7, 4 to 8, 4 to 9, 4 to 10, 4 to 11, 4 to 12, 5 to 6, 5 to 7, 5 to 8, 5 to 9, 5 to 10, 5 to 11, 5 to 12, 6 to 7, 6 to 8, 6 to 9, 6 to 10, 6 to 11, 6 to 12, 7 to 8, 7 to 9, 7 to 10, 7 to 11, 7 to 12, 8 to 9, 8 to 10, 8 to 11, 8 to 12, 9 to 10, 9 to 11, 9 to 12, 10 to 11, 10 to 12, 11 to 12, 2, 3, 4, 5, 6, 7, 8, 9, 10, 11, 12). In some embodiments, y is 2 to 8 (e.g., 2 to 3, 2 to 4, 2 to 5, 2 to 6, 2 to 7, 3 to 4, 3 to 5, 3 to 6, 3 to 7, 3 to 8, 4 to 5, 4 to 6, 4 to 7, 4 to 8, 5 to 6, 5 to 7, 5 to 8, 6 to 7, 6 to 8, 7 to 8, 2, 3, 4, 5, 6, 7, 8). In some embodiments, z is 2 to 14 (e.g., 2 to 3, 2 to 4, 2 to 5, 2 to 6, 2 to 7, 2 to 8, 2 to 9, 2 to 10, 2 to 11, 2 to 12, 2 to 13, 3 to 4, 3 to 5, 3 to 6, 3 to 7, 3 to 8, 3 to 9, 3 to 10, 3 to 11, 3 to 12, 3 to 13, 3 to 14, 4 to 5, 4 to 6, 4 to 7, 4 to 8, 4 to 9, 4 to 10, 4 to 11, 4 to 12, 4 to 13, 4 to 14, 5 to 6, 5 to 7, 5 to 8, 5 to 9, 5 to 10, 5 to 11, 5 to 12, 5 to 13, 5 to 14, 6 to 7, 6 to 8, 6 to 9, 6 to 10, 6 to 11, 6 to 12, 6 to 13, 6 to 14, 7 to 8, 7 to 9, 7 to 10, 7 to 11, 7 to 12, 7 to 13, 7 to 14, 8 to 9, 8 to 10, 8 to 11, 8 to 12, 8 to 13, 8 to 14, 9 to 10, 9 to 11, 9 to 12, 9 to 13, 9 to 14, 10 to 11, 10 to 12, 10 to 13, 10 to 14, 11 to 12, 11 to 13, 11 to 14, 12 to 13, 12 to 14, 13 to 14, 2, 3, 4, 5, 6, 7, 8, 9, 10, 11, 12, 13, 14).

In some embodiments, the composition includes nanostructured terephthalic acid. Without wishing to be bound by theory, it is believed that nanostructured terephthalic acid differs from other forms of terephthalic acid due to the unique nanostructured morphology (see discussion below and Examples).

In certain embodiments, the composition consists of the nanostructured organic compound. In certain embodiments, the composition consists of the nanostructured terephthalic acid.

In some embodiments, the composition is nanostructured and nanocrystalline. A composition is said to be nanocrystalline if it includes crystalline domain sizes smaller than 100 nm (e.g. below 95 below 90, below 85, below 80, below 75, below 70, below 65, below 60, below 55, below 50, below 45, below 40, below 35, below 30, below 25, below 20, below 15, below 10, below 5, below 4, below 3, below 2, below 1) nm. In some embodiments, the organic compound is nanocrystalline.

In certain embodiments, the composition includes a metal oxide, a metal, a metal-organic framework, a silicon-containing material and/or a graphene-containing material embedded within the nanostructured organic compound (see discussion below).

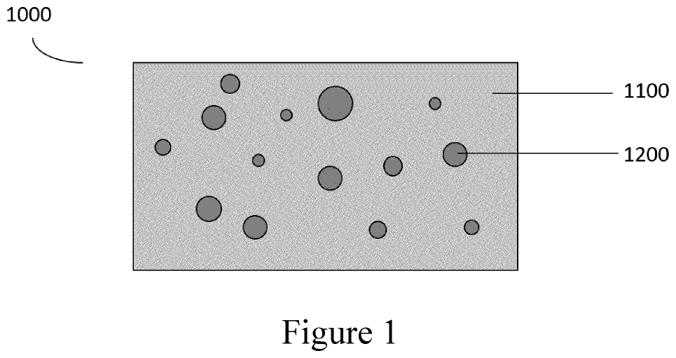

FIG. 1 schematically illustrates an embodiment of a composition 1000 of the disclosure. The composition 1000 includes an at least partially crystalline organic compound (e.g., terephthalic acid) 1100 and a crystalline metal oxide (e.g., tin oxide (SnO₂)) 1200 is dispersed in the organic compound 1100. In some embodiments, the composition 1000 is nanostructured and/or nanocrystalline.

In general, the organic compound (e.g., the nanostructured organic compound, the organic compound 1100) has the formula CₓO_{y}H_{z}. In some embodiments, x is 2 to 12 (e.g., 2 to 3, 2 to 4, 2 to 5, 2 to 6, 2 to 7, 2 to 8, 2 to 9, 2 to 10, 2 to 11, 3 to 4, 3 to 5, 3 to 6, 3 to 7, 3 to 8, 3 to 9, 3 to 10, 3 to 11, 3 to 12, 4 to 5, 4 to 6, 4 to 7, 4 to 8, 4 to 9, 4 to 10, 4 to 11, 4 to 12, 5 to 6, 5 to 7, 5 to 8, 5 to 9, 5 to 10, 5 to 11, 5 to 12, 6 to 7, 6 to 8, 6 to 9, 6 to 10, 6 to 11, 6 to 12, 7 to 8, 7 to 9, 7 to 10, 7 to 11, 7 to 12, 8 to 9, 8 to 10, 8 to 11, 8 to 12, 9 to 10, 9 to 11, 9 to 12, 10 to 11, 10 to 12, 11 to 12, 2, 3, 4, 5, 6, 7, 8, 9, 10, 11, 12). In some embodiments, y is 2 to 8 (e.g., 2 to 3, 2 to 4, 2 to 5, 2 to 6, 2 to 7, 3 to 4, 3 to 5, 3 to 6, 3 to 7, 3 to 8, 4 to 5, 4 to 6, 4 to 7, 4 to 8, 5 to 6, 5 to 7, 5 to 8, 6 to 7, 6 to 8, 7 to 8, 2, 3, 4, 5, 6, 7, 8). In some embodiments, z is 2 to 14 (e.g., 2 to 3, 2 to 4, 2 to 5, 2 to 6, 2 to 7, 2 to 8, 2 to 9, 2 to 10, 2 to 11, 2 to 12, 2 to 13, 3 to 4, 3 to 5, 3 to 6, 3 to 7, 3 to 8, 3 to 9, 3 to 10, 3 to 11, 3 to 12, 3 to 13, 3 to 14, 4 to 5, 4 to 6, 4 to 7, 4 to 8, 4 to 9, 4 to 10, 4 to 11, 4 to 12, 4 to 13, 4 to 14, 5 to 6, 5 to 7, 5 to 8, 5 to 9, 5 to 10, 5 to 11, 5 to 12, 5 to 13, 5 to 14, 6 to 7, 6 to 8, 6 to 9, 6 to 10, 6 to 11, 6 to 12, 6 to 13, 6 to 14, 7 to 8, 7 to 9, 7 to 10, 7 to 11, 7 to 12, 7 to 13, 7 to 14, 8 to 9, 8 to 10, 8 to 11, 8 to 12, 8 to 13, 8 to 14, 9 to 10, 9 to 11, 9 to 12, 9 to 13, 9 to 14, 10 to 11, 10 to 12, 10 to 13, 10 to 14, 11 to 12, 11 to 13, 11 to 14, 12 to 13, 12 to 14, 13 to 14, 2, 3, 4, 5, 6, 7, 8, 9, 10, 11, 12, 13, 14).

Examples of the organic compound (e.g., the nanostructured organic compound, the organic compound 1100) include terephthalic acid, terephthalate, dimethyl terephthalate, Bis(2-Hydroxyethyl) terephthalate, ethylene glycol, phthalic acid, protocatechuic acid, and isophthalic acid.

In certain embodiments, the organic compound 1100 is partially crystalline, i.e., contains an amorphous phase and a crystalline phase. In certain embodiments, the organic compound 1100 is crystalline and does not contain an amorphous phase.

In certain embodiments, the organic compound 1100 is nanostructured. In general, in such embodiments, the organic compound 1100 forms clusters with a size of at least 1 (e.g., at least 2, at least 5, at least 10, at least 20, at least 50) nanometers (nm) and/or at most 200 (e.g., at most 150, at most 100) nm.

In some embodiments, a composition according to the disclosure (e.g., a nanostructured organic compound, the composition 1000) includes at least 1 (e.g., at least 2, at least 3, at least 4, at least 5) distinct organic compounds (see Examples 17 and 18). In some embodiments, at least a portion (e.g., all) of the distinct organic compounds are at least partially crystalline. In some embodiments, at least a portion (e.g., all) of the distinct organic compounds are crystalline. In some embodiments, at least a portion (e.g., all) of the distinct organic compounds are nanostructured.

In certain embodiments, the composition 1000 contains at least 5 (e.g., at least 10, at least 15, at least 20, at least 25, at least 30, at least 35, at least 40, at least 45, at least 50) wt. % and/or at most 99 (e.g., at most 95, at most 90, at most 85, at most 80, at most 75, at most 70, at most 65, at most 60, at most 55, at most 50) wt. % of the organic compound (e.g., terephthalic acid) 1100.

Examples of the crystalline metal oxide 1200 include tin oxides (e.g., tin(IV) oxide (SnO₂), tin(II) oxide (SnO)), zinc oxides (e.g., zinc oxide (ZnO), zinc peroxide (ZnO₂)), calcium oxides, lithium oxides, potassium oxides, lead oxides, iron oxides, molybdenum oxides, cobalt oxides, chromium oxides, niobium oxides, and manganese oxides. In some embodiments, the crystalline metal oxide 1200 includes a semimetal such as a germanium oxide or silicon oxide. In some embodiments, the crystalline metal oxide 1200 includes Sn, Fe, Mo, Co, Cr, Nb, Mn, Zn, Ge and/or Si.

In some embodiments, the composition 1000 contains at least 1 (e.g., at least 5, at least 10, at least 15, at least 20, at least 25, at least 30, at least 35, at least 40, at least 45, at least 50) wt. % and/or at most 95 (e.g., at most 90, at most 85, at most 80, at most 75, at most 70, at most 65, at most 60, at most 55, at most 50) w.t. % of the crystalline metal oxide (e.g., SnO₂) 1200.

In some embodiments, the crystalline metal oxide 1200 forms nanoparticles. In such embodiments, the crystalline metal oxide 1200 has a particle size of at least 1 (e.g., at least 2, at least 3, at least 4, at least 5) nm and/or at most 100 (e.g. at most 50, at most 20, at most 10, at most 5) nm.

In some embodiments, an amount of the crystalline metal oxide 1200 in an interior region of the composition (i.e., in the bulk) 1000 is greater than an amount of crystalline metal oxide 1200 at the surface of the material 1000. In such embodiments, the amount of crystalline metal oxide 1200 in the interior region of the composition 1000 (i.e. a bulk amount) is at least 1 (e.g., at least 5, at least 10, at least 15, at least 20, at least 25, at least 30, at least 35, at least 40, at least 45, at least 50) wt. % and/or at most 95 (e.g., at most 90, at most 85, at most 80, at most 75, at most 70, at most 60, at most 50) wt. %, and/or the amount of crystalline metal oxide 1200 at the surface of the composition 1000 is at least 0.1 (e.g. at least 0.5, at least 1, at least 2, at least 5, at least 10, at least 15, at least 20) wt. % and/or at most 80 (e.g. at most 70, at most 60, at most 50) wt. %. Without wishing to be bound by theory, it is believed that the crystalline metal oxide (e.g., SnO₂) 1200 particles are covered by layers of the organic compound (e.g., terephthalic acid) 1100, resulting in the incorporation of the crystalline metal oxide 1200 particles into the bulk organic compound 1100 and therefore a greater amount of the of crystalline metal oxide 1200 being present in the bulk, relative to the surface of the composition 1000.

In certain embodiments, a composition of the disclosure (e.g., a composition containing a nanostructured organic compound, the composition 1000) has crystalline domain sizes of at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95) nm and/or at most 100 (e.g., at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 9, at most 8, at most 7, at most 6, at most 5, at most 4, at most 3, at most 2) nm.

A composition of the disclosure (e.g., a composition containing a nanostructured organic compound, the composition 1000) can contain nanoparticles with sizes of 1 (e.g., at least 10, at least 20, at least 50, at least 100, at least 150) nm and/or at most 200 (e.g., at most 100, at most 50, at most 20, at most 10) nm.

A composition of the disclosure (e.g., a composition containing a nanostructured organic compound, the composition 1000) can form particles with a size of at least 0.01 (e.g., at least 0.1, at least 1, at least 5, at least 10, at least 50 at least 100) μm and/or at most 100 (e.g., at most 50, at most 10, at most 5, at most 1) μm. In certain embodiments, the particles include nanoparticles with a size of at least 1 (e.g., at least 10, at least 20, at least 50, at least 100, at least 150) nm and/or at most 200 (e.g., at most 100, at most 50, at most 20, at most 10) nm. In certain embodiments the particles include sheet-like particles with sizes of 1 (e.g., at least 5, at least 10, at least 50, at least 100, at least 200 at least 500) nm and/or at most 1000 (e.g., at most 500, at most 200, at most 100, at most 50, at most 10, at most 5) nm.

In certain embodiments, composition of the disclosure (e.g., a composition containing a nanostructured organic compound, the composition 1000) has a surface area of at least 10 (e.g., at least 15, at least 17, at least 20) square meters per gram (m² g⁻¹) and/or at most 50 (e.g. at most 45, at most 40, at most 35, at most 30, at most 25, at most 21, at most 20) m² g⁻¹. Measurement of the surface area is described in Example 8 below.

In some embodiments, composition of the disclosure (e.g., a composition containing a nanostructured organic compound, the composition 1000) has a bulk electrical conductivity of at least 5 (e.g., at least 10, at least 20, at least 50, at least 100, at least 150, at least 200, at least 250, at least 300, at least 350, at least 400, at least 450, at least 500) Siemens per meter (S m⁻¹) and/or at most 5000 (e.g., at most 4000, at most 3000, at most 2000, at most 1500, at most 1000, at most 900, at most 800, at most 700, at most 600, at most 500) S m⁻¹ at 6.3 MPa. Measurement of the bulk electrical conductivity is described in Example 10 below. Without wishing to be bound by theory, it is believed that the relatively high electrical conductivity of the composition 1000 is due to the presence of crystalline metal oxide (e.g., SnO₂) 1200 particles. For example, although the crystalline metal oxide (e.g., SnO₂) 1200 is a semiconductor, it can still exhibit metallic conductivity for various reasons, including the presence of oxygen vacancies.

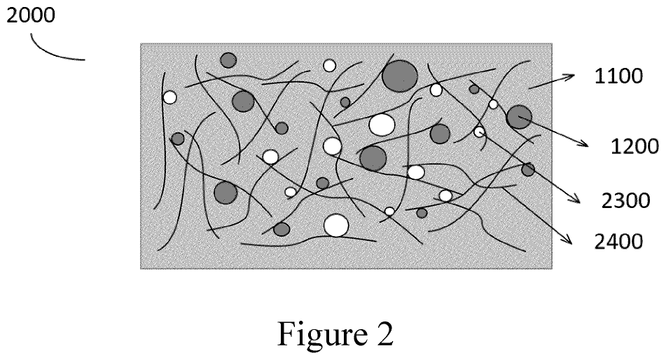

FIG. 2 schematically illustrates an embodiment of a composition 2000 of the disclosure. The composition 2000 includes the components of the composition 1000 in FIG. 1 as well as a silicon-containing material 2300 and graphene nanosheets 2400 dispersed in the organic compound 1100. Without wishing to be bound by theory, it is believed that the silicon-containing material 2300 is embedded in the organic compound 1100.

In some embodiments, a nanostructured organic compound (e.g., nanostructured terephthalic acid) includes a silicon-containing material and graphene nanosheets dispersed in the nanostructured organic compound.

In certain embodiments, a composition according to the disclosure (e.g., a nanostructured organic compound, the composition 2000) contains at least 0.1 (e.g., at least 0.5, at least 1, at least 5, at least 10) wt. % and/or at most 95 (e.g., at most 90, at most 85) wt. % silicon-containing material 2300. In certain embodiments, the silicon-containing material 2300 is elemental silicon. In certain embodiment, the silicon-containing material 2300 is SiNPs. In certain embodiments, the silicon-containing material 2300 has a size of at least 1 (e.g., at least 2, at least 3, at least 4, at least 5) nm and/or at most 1000 (e.g., at most 500, at most 200, at most 100, at most 50, at most 20, at most 10, at most 5) nm.

In some embodiments, a composition according to the disclosure (e.g., a nanostructured organic compound, the composition 2000) contains at least 0.1 (e.g., at least 0.5, at least 1, at least 5, at least 10) wt. % and/or at most 50 (e.g., at most 45, at most 40) wt. % graphene nanosheets 2400. In some embodiments, the graphene nanosheets 2400 contain flakes having a flake size of at least 1 (e.g., at least 2, at least 5, at least 10, at least 20, at least 50, at least 100) nm and/or at most 5 (e.g., at most 4, at most 3, at most 2, at most 1) μm. In some embodiments, the graphene nanosheets 2400 have at least 1 (e.g., at least 2, at least 3, at least 4, at least 5) layers and/or at most 100 (e.g., at most 90, at most 80, at most 70, at most 60, at most 50, at most 40, at most 30, at most 20, at most 10) layers. In some embodiments, the graphene nanosheets 2400 have a carbon purity of at least 90 (e.g., at least 91, at least 92, at least 93, at least 94, at least 95) %. In some embodiments, the graphene nanosheets 2400 contain functional groups (e.g., hydroxyl group, a carbonyl group, a carboxyl group and/or an amino group) on their surface.

In certain embodiments, the graphene nanosheets 2400 cover at least a portion of the crystalline metal oxide 1200 and/or the silicon-containing material 2300.

While FIG. 2 schematically illustrates an embodiment, in some embodiments, the composition 2000 contains a silicon-containing material 2300, in addition to the organic compound 1100 and the crystalline metal oxide 1200, but does not contain graphene nanosheets 2400. Further, in certain embodiments, the composition 2000 contains graphene nanosheets 2400, in addition to the organic compound 1100 and the crystalline metal oxide 1200, but does not contain a silicon-containing material 2300.

A composition according to the disclosure can include an organic compound and a metal-organic framework embedded within the organic compound. In some embodiments, the organic compound is at least partially crystalline. In some embodiments, the organic compound is crystalline. In some embodiments, the organic compound is nanostructured. In some embodiments, the metal-organic framework is at least partially crystalline. In some embodiments, the metal-organic framework is crystalline. In some embodiments, the metal-organic framework is nanostructured. In some embodiments, the metal-organic framework is nanocrystalline.

In certain embodiments, a composition of the disclosure (e.g., the nanostructured organic compound, the composition 1000, the composition 2000) can include a metal-organic framework embedded within the organic compound.

In certain embodiments, the metal-organic framework includes the organic compound and a metal.

In some embodiments, the metal-organic framework has the formula MCₓH_{y}O_{z}·nH₂O, wherein M is a metal and n is 0 to 5. In some embodiments, n is 0, 1, 2 or 3.5. In some embodiments, the metal-organic framework has the formula MCₓH_{y}O_{z}·3.5H₂O. In some embodiments, the metal-organic framework has the formula MCₓH_{y}O_{z}. In some embodiments, x is 2 to 12 (e.g., 2 to 3, 2 to 4, 2 to 5, 2 to 6, 2 to 7, 2 to 8, 2 to 9, 2 to 10, 2 to 11, 3 to 4, 3 to 5, 3 to 6, 3 to 7, 3 to 8, 3 to 9, 3 to 10, 3 to 11, 3 to 12, 4 to 5, 4 to 6, 4 to 7, 4 to 8, 4 to 9, 4 to 10, 4 to 11, 4 to 12, 5 to 6, 5 to 7, 5 to 8, 5 to 9, 5 to 10, 5 to 11, 5 to 12, 6 to 7, 6 to 8, 6 to 9, 6 to 10, 6 to 11, 6 to 12, 7 to 8, 7 to 9, 7 to 10, 7 to 11, 7 to 12, 8 to 9, 8 to 10, 8 to 11, 8 to 12, 9 to 10, 9 to 11, 9 to 12, 10 to 11, 10 to 12, 11 to 12, 2, 3, 4, 5, 6, 7, 8, 9, 10, 11, 12). In some embodiments, y is 2 to 8 (e.g., 2 to 3, 2 to 4, 2 to 5, 2 to 6, 2 to 7, 3 to 4, 3 to 5, 3 to 6, 3 to 7, 3 to 8, 4 to 5, 4 to 6, 4 to 7, 4 to 8, 5 to 6, 5 to 7, 5 to 8, 6 to 7, 6 to 8, 7 to 8, 2, 3, 4, 5, 6, 7, 8). In some embodiments, z is 2 to 14 (e.g., 2 to 3, 2 to 4, 2 to 5, 2 to 6, 2 to 7, 2 to 8, 2 to 9, 2 to 10, 2 to 11, 2 to 12, 2 to 13, 3 to 4, 3 to 5, 3 to 6, 3 to 7, 3 to 8, 3 to 9, 3 to 10, 3 to 11, 3 to 12, 3 to 13, 3 to 14, 4 to 5, 4 to 6, 4 to 7, 4 to 8, 4 to 9, 4 to 10, 4 to 11, 4 to 12, 4 to 13, 4 to 14, 5 to 6, 5 to 7, 5 to 8, 5 to 9, 5 to 10, 5 to 11, 5 to 12, 5 to 13, 5 to 14, 6 to 7, 6 to 8, 6 to 9, 6 to 10, 6 to 11, 6 to 12, 6 to 13, 6 to 14, 7 to 8, 7 to 9, 7 to 10, 7 to 11, 7 to 12, 7 to 13, 7 to 14, 8 to 9, 8 to 10, 8 to 11, 8 to 12, 8 to 13, 8 to 14, 9 to 10, 9 to 11, 9 to 12, 9 to 13, 9 to 14, 10 to 11, 10 to 12, 10 to 13, 10 to 14, 11 to 12, 11 to 13, 11 to 14, 12 to 13, 12 to 14, 13 to 14, 2, 3, 4, 5, 6, 7, 8, 9, 10, 11, 12, 13, 14).

Examples of the metal include Zn, Fe, Cu, Al, Zr, Cr, Co, Li, Na and K.

In some embodiments, the composition forms particles with a size of at least 0.5 (e.g., at least 1, at least 5, at least 10, at least 15, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550) μm and/or at most 600 (e.g., at most 550, at most 500, at most 450, at most 400, at most 350, at most 300, at most 250, at most 200, at most 150, at most 100, at most 90, at most 80, at most 70, at most 60, at most 50, at most 40, at most 30, at most 20, at most 15, at most 10, at most 5, at most 1) μm. In some embodiments, the particles include sheet-like particles with sizes of at least 10 (e.g., at least 20, at least 50, at least 100, at least 500, at least 1000, at least 5000) nm and/or at most 10000 (e.g., at most 5000, at most 1000, at most 500, at most 100, at most 50, at most 20) nm. In some embodiments, the sheet-like particles have a thickness of at least 1 (e.g., at least 2, at least 3, at least 4, at least 5, at least 6, at least 7, at least 8, at least 9) nm and/or at most 10 (e.g., at most 9, at most 8, at most 7, at most 6, at most 5, at most 4, at most 3, at most 2) nm. In some embodiments, the sheet-like particles include nanoparticles with sizes of at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 55) nm and/or at most 60 (e.g., at most 55, at most 50, at most 45, at most 40, at most 35, at most 30, at most 25, at most 20, at most 15, at most 10, at most 9, at most 8, at most 7, at most 6, at most 5, at most 4, at most 3, at most 2) nm. In some embodiments, the particles include agglomerated nanoparticles with sizes of at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 55) nm and/or at most 60 (e.g., at most 55, at most 50, at most 45, at most 40, at most 35, at most 30, at most 25, at most 20, at most 15, at most 10, at most 9, at most 8, at most 7, at most 6, at most 5, at most 4, at most 3, at most 2) nm. In some embodiments, the particles include metal-organic framework crystals with sizes of at least 10 (e.g., at least 50, at least 100, at least 500, at least 1000, at least 5000) nm and/or at most 10000 (e.g., at most 5000, at most 1000, at most 500, at most 100, at most 50) nm.

In some embodiments, the metal-organic framework has an average crystalline domain size of at least 30 (e.g., at least 35, at least 40, at least 45, at least 50, at least 55) nm and/or at most 60 (e.g., at least 55, at least 50, at least 45, at least 40, at least 35 nm).

In certain embodiments, a composition of the disclosure (e.g., the nanostructured organic compound, the composition 1000, the composition 2000) can include a tin-containing member. Examples of the tin-containing member include metallic tin, tin chloride, tin chloride hydrate, tin chloride hydroxide and tin oxide chloride hydroxide. In certain embodiments, the tin oxide chloride hydroxide has the formula Sn₂₁Cl₁₆(OH)₁₄O₆. Without wishing to be bound by theory, it is believed that of tin oxide chloride hydroxide can be formed during the hydrolysis of SnCl₂ during a solvent contacting step (see discussion below), such as washing with distilled water, as shown in the reaction below

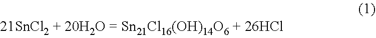

Tin oxide chloride hydroxide has a relatively low solubility in water, and therefore, remains in the organic compound (e.g., nanostructure of terephthalic acid) after washing and filtration.

In some embodiments, the tin-containing member is at least partially crystalline. In some embodiments, the tin-containing member is crystalline. In some embodiments, the tin-containing member is nanostructured. In some embodiments, the tin-containing member forms particles with a size of at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95) nm and/or at most 100 (e.g., at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 9, at most 8, at most 7, at most 6, at most 5, at most 4, at most 3, at most 2) nm.

In certain embodiments, a composition of the disclosure (e.g., the nanostructured organic compound, the composition 1000, the composition 2000) can include a metal. Examples of the metal include Sn, Zn, Fe, Cu, Ni, Cr, Al and Co.

In some embodiments, a composition of the disclosure (e.g., the nanostructured organic compound, the composition 1000, the composition 2000) has an absorbance of at least 1 (e.g., at least 1.1, at least 1.2, at least 1.3, at least 1.4, at least 1.5, at least 1.6, at least 1.7, at least 1.8, at least 1.9) a.u. and/or at most 2 (e.g., at most 1.9, at most 1.8, at most 1.7, at most 1.6, at most 1.5, at most 1.4, at most 1.3, at most 1.2, at most 1.1) a.u. at 242 nm at a concentration of 0.5 g/L. In some embodiments, a composition of the disclosure (e.g., the nanostructured organic compound, the composition 1000, the composition 2000) has an absorbance of at least 0.1 (e.g., at least 0.2, at least 0.3, at least 0.4, at least 0.5, at least 0.6, at least 0.7) a.u. and/or at most 0.8 (e.g., at most 0.7, at most 0.6, at most 0.5, at most 0.4, at most 0.3, at most 0.2) a.u., where absorbance is measured at 450 nm at a concentration of 0.5 g/L. In some embodiments, a composition of the disclosure (e.g., the nanostructured organic compound, the composition 1000, the composition 2000) has an absorbance of at least 0.1 (e.g., at least 0.2, at least 0.3, at least 0.4, at least 0.5, at least 0.6, at least 0.7) a.u. and/or at most 0.8 (e.g., at most 0.7, at most 0.6, at most 0.5, at most 0.4, at most 0.3, at most 0.2) a.u., where absorbance is measured at 500 nm at a concentration of 0.5 g/L. In some embodiments, a composition of the disclosure (e.g., the nanostructured organic compound, the composition 1000, the composition 2000) has an absorbance of at least 0.1 (e.g., at least 0.2, at least 0.3, at least 0.4, at least 0.5, at least 0.6, at least 0.7) a.u. and/or at most 0.8 (e.g., at most 0.7, at most 0.6, at most 0.5, at most 0.4, at most 0.3, at most 0.2) a.u., where absorbance is measured at 317 nm at a concentration of 0.5 g/L.

In some embodiments, a composition of the disclosure (e.g.: a composition including an at least partially crystalline organic compound a metal oxide; a composition including a nanostructured organic compound that includes a plurality of molecules having the formula CₓO_{y}H_{z}; a composition including an organic compound and a metal-organic framework embedded within the organic compound; or a composition including an organic compound and a crystalline metal oxide) further includes a transition metal dichalcogenide. Examples of such transition metal dichalcogenides include those having the empirical formula MX₂, where M is a transition metal atom (such as Mo or W) and X is a chalcogen atom (such as S, Se or Te). Without wishing to be bound by theory, it is believed that in some embodiments, the molten salt (see discussion below) can protect the transition metal dichalcogenide from oxidation at temperatures greater than 300° C., where these compounds would normally undergo oxidation. In some embodiments, the transition metal dichalcogenide is a two dimensional transition metal dichalcogenide. In some embodiments, the transition metal dichalcogenide is embedded within the organic compound and/or metal oxide (when present), which, without wishing to be bound by theory, it is believed to enhance the integrity and conductivity of the composition. Without wishing to be bound by theory, it is believed that this can result in enhanced kinetics of metal-ion insertion and extraction into and out of an electrode containing such as composition. In some embodiments, the presence of one or more transition metal dichalcogenides within a composition according to the disclosure can increase the rate capability of the resulting electrode for metal-ion battery application, such as the electrode for Li-ion battery, Na-ion battery and K-ion battery (see discussion below).

### Electrodes and Energy Storage Devices

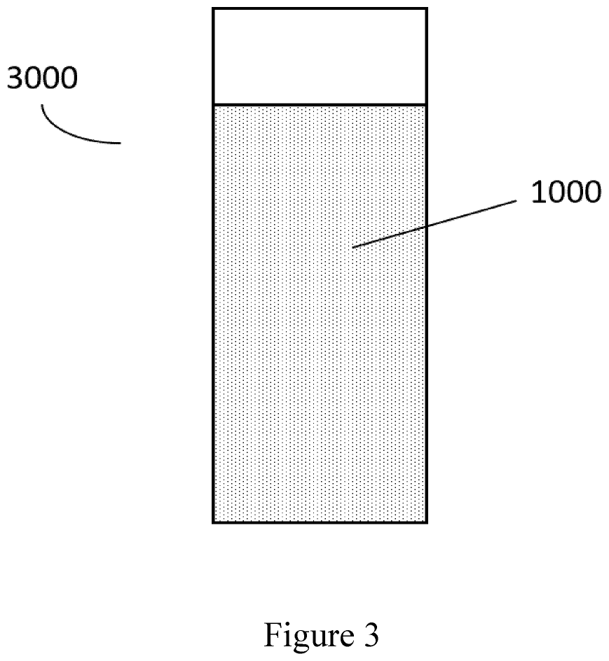

As schematically depicted in FIG. 3, the composition 1000 can be used to make an electrode 3000, such as an anode. While FIG. 3 schematically illustrates an embodiment, in some embodiments, the electrode 3000 contains the composition 2000, the nanostructured organic compound, and/or a composition containing an organic compound and a metal-organic framework embedded within the organic compound instead of or in addition to the composition 1000. In some embodiments, in addition to the composition 1000, the composition 2000, the nanostructured organic compound, and/or a composition containing an organic compound and a metal-organic framework embedded within the organic compound, the electrode 3000 contains a binder (e.g., polyvinylidene fluoride (PVDF), styrene-butadiene rubber (SBR), carboxymethyl cellulose (CMC), polyacrylic acid (PAA), polytetrafluorethylene, polyvinyl alcohol (PVA), poly-glutamic acid (PGA), sodium alginate (SA), chitosan (CS), polyacrylonitrile (PAN), polyimide (PI), gum), a solvent (e.g., N-methyl-2-pyrrolidone (NMP), water), conductive carbon, copper foil and/or graphene nanosheets. Optionally, the electrode 3000 can contain one or more additional constituents as appropriate.

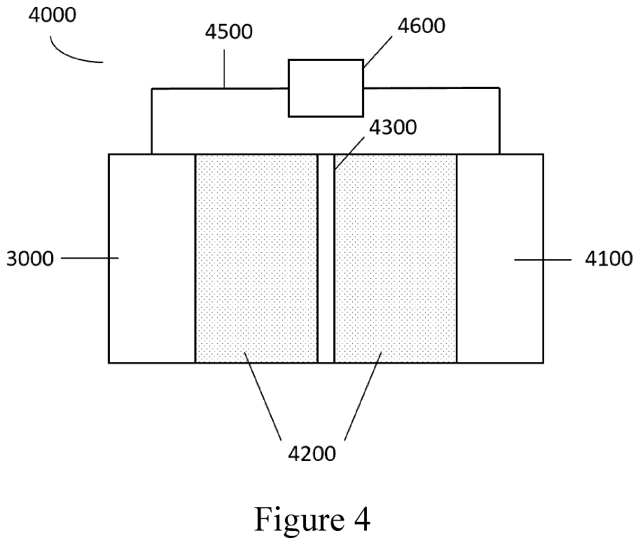

In general, the electrode 3000 can be used in an energy storage device, such as a battery or a supercapacitor. As an example, FIG. 4 shows a single cell of a battery 4000 including the electrode 3000 as an anode, a cathode 4100, an electrolyte 4200, and a separator 4300 between the anode 3000 and cathode 4100. The depicted cell of the battery 4000 also includes a wire 4500 and a load 4600 connecting the anode 3000 and the cathode 4100. The battery 4000 includes a plurality of such cells. Examples of batteries include lithium-ion batteries, sodium-ion batteries, calcium-ion batteries, and potassium-ion batteries.

Without wishing to be bound by theory, it is believed that the crystalline metal oxide (e.g., SnO₂) 1200 can be an active material in the electrode 3000. Furthermore, without wishing to be bound by theory, it is believed that the presence of the organic compound (e.g., terephthalic acid) 1100 can reduce (e.g. prevent) the disintegration of crystalline metal oxide (e.g., SnO₂) 1200 relative to the absence of the organic compound (e.g., terephthalic acid) 1100, as the organic compound (e.g., terephthalic acid) 1100 can support the integrity of the electrode 3000, can reduce (e.g., prevent) degradation of the crystalline metal oxide (e.g., SnO₂) 1200, and/or can maintain good contact between particles of the crystalline metal oxide (e.g., SnO₂) 1200. Without wishing to be bound by theory, it is believed that these properties may result, at least in part, from hydrogen bonding between the crystalline metal oxide 1200 and organic compound 1100 (e.g., between the oxygen of SnO₂ and hydrogen of terephthalic acid).

Without wishing to be bound by theory, it is believed that the compositions of the disclosure (e.g., the composition 1000 and/or the composition 2000), such as when in the form of the electrode 3000, can have desirable properties due, at least in part, to the reduced charge-transfer resistance from the uniform distribution of crystalline metal oxide (e.g., SnO₂) 1200 particles within the organic compound (e.g., terephthalic acid) 1100 matrix.

In certain embodiments, the electrode 3000 has a lithium-ion (“Li-ion”) discharge capacity of at least 10 (e.g., at least 20, at least 50, at least 100, at least 150, at least 200, at least 250, at least 300, at least 350, at least 400, at least 450, at least 500) milliAmpere hours per gram (mAh g⁻¹) and/or at most 1500 (e.g., at most 1400, at most 1300, at most 1200, at most 1100, at most 1000, at most 950, at most 900, at most 850, at most 800, at most 750, at most 700, at most 650, at most 600, at most 550, at most 500) mAh g⁻¹ after 500 cycles at a current density of 200 mA g⁻¹. In certain embodiments, the electrode has a Li-ion discharge capacity of at least 100 (e.g., 200, at least 500) milliAmpere hours (mAh) per gram of the crystalline metal oxide to at most 1800 (e.g., at most 1700, at most 1600) mAh per gram of crystalline metal oxide after 500 cycles. Measurement of the lithium-ion discharge capacity is described in Example 11 below.

In certain embodiments, the electrode 3000 has a Li-ion discharge capacity of at least 300 (e.g., at least 400, at least 500, at least 600, at least 700, at least 800, at least 900, at least 1000) mAh g⁻¹ and/or at most 1500 (e.g., at most 1400, at most 1300, at most 1200, at most 1100, at most 1000) mAh g⁻¹ after 10 cycles at a current density of 100 mA g⁻¹. In certain embodiments, the electrode 3000 has a Li-ion discharge capacity of at least 200 (e.g., at least 300, at least 400, at least 500, at least 600, at least 700, at least 800, at least 900, at least 1000) mAh g⁻¹ and/or at most 1500 (e.g., at most 1400, at most 1300, at most 1200, at most 1100, at most 1000) mAh g⁻¹ after 30 cycles at a current density of 500 mA g⁻¹. In certain embodiments, the electrode 3000 has a Li-ion discharge capacity of at least 200 (e.g., at least 300, at least 400, at least 500, at least 600, at least 700, at least 800, at least 900, at least 1000) mAh g⁻¹ and/or at most 1500 (e.g., at most 1400, at most 1300, at most 1200, at most 1100, at most 1000) mAh g⁻¹ after 50 cycles at a current density of 1000 mA g⁻¹. In certain embodiments, the electrode 3000 has a Li-ion discharge capacity of at least 50 (e.g., at least 100, at least 200, at least 300, at least 400, at least 500, at least 600, at least 700, at least 800, at least 900, at least 1000) mAh g⁻¹ and/or at most 1300 (e.g., at most 1200, at most 1100, at most 1000) mAh g⁻¹ after 60 cycles at a current density of 5000 mA g⁻¹. In certain embodiments, the electrode has a Li-ion discharge capacity of at least 100 (e.g., at least 200, at least 300, at least 400, at least 500, at least 600, at least 700, at least 800, at least 900, at least 1000, at least 1100, at least 1200, at least 1300, at least 1400, at least 1500, at least 1600, at least 1700) mAh g⁻¹ and/or at most 1800 (e.g., at most 1700, at most 1600, at most 1500, at most 1400, at most 1300, at most 1200, at most 1100, at most 1000, at most 900, at most 800, at most 700, at most 600, at most 500, at most 400, at most 300, at most 200) mAh per g⁻¹ after 500 cycles.

Without wishing to be bound by theory, it is believed that the lithium-ion discharge capacity of the electrode 3000 is due, at least in part, to a relatively small average size of crystalline metal oxide (e.g., SnO₂) 1200 particles and the presence of the organic compound (e.g., terephthalic acid) 1100, which impact the properties of the electrode as described above.

In some embodiments, the electrode has a coulombic efficiency of at least 70 (e.g., at least 75, at least 80, at least 85, at least 90, at least 95) % and/or at most 120 (e.g. at most 115, at most 100, at most 105, at most 100) % after 500 cycles. Measurement of the coulombic efficiency is described in Example 11 below.

Without wishing to be bound by theory, it is believed that during lithiation-dilithiation cycles, the reactions occurring are:

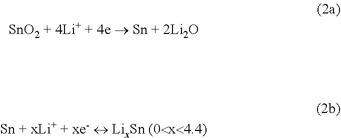

In some embodiments, a half-cell containing the electrode 3000 and an electrolyte has an increase in electrolyte resistance of at least 1.0 (e.g., at least 1.5, at least 2.0, at least 2.5, at least 3.0, at least 3.5, at least 4.0) Ω and/or at most 6.0 (e.g., at most 5.5, at most 5.0, at most 4.5, at most 4.0, at most 3.5, at most 3.0) Ω after 150 cycles. In some embodiments, a half-cell containing the electrode 3000 and an electrolyte has an increase in electrolyte resistance of at least 2.0 (e.g., at least 2.5, at least 3.0, at least 3.5, at least 4.0) Ω and/or at most 8.0 (e.g., at most 7.5, at most 7.0, at most 6.5, at most 6.0, at most 5.5, at most 5.0, at most 4.5, at most 4.0, at most 3.5, at most 3.0) Ω after 300 cycles. Measurement of the electrolyte resistance is described in Example 11 below.

In certain embodiments, the electrode 3000 has a lithium ion (“Li-ion”) diffusion rate of at least 10⁻¹¹ (e.g., at least 2×10⁻¹¹, at least 3×10⁻¹¹, at least 4×10⁻¹¹, at least 5×10⁻¹¹, at least 6×10⁻¹¹, at least 7×10⁻¹¹, at least 8×10⁻¹¹ at least 9×10⁻¹¹) cm²s⁻¹ and/or at most 9×10⁹ (e.g., at most 8×10⁻⁹, at most 7×10⁻⁹, at most 6×10⁻⁹, at most 5×10⁻⁹, at most 4×10⁻⁹, at most 3×10⁻⁹, at most 2×10⁻⁹ at most 10⁻⁹) cm²s⁻¹ after 150 cycles. In certain embodiments, the electrode 3000 has a lithium ion diffusion rate of at least 2×10⁻¹¹ (e.g., at least 3×10⁻¹¹, at least 4×10⁻¹¹, at least 5×10⁻¹¹, at least 6×10⁻¹¹, at least 7×10⁻¹¹, at least 8×10⁻¹¹ at least 9×10⁻¹¹, at least 10⁻¹⁰) cm²s⁻¹ and/or at most 10⁻⁸ (e.g., at most 9×10⁻⁹, at most 8×10⁻⁹, at most 7×10⁻⁹, at most 6×10⁻⁹, at most 5×10⁻⁹, at most 4×10⁻⁹, at most 3×10⁻⁹, at most 2×10⁻⁹ at most 10⁻⁹) cm²s⁻¹ after 300 cycles. Measurement of the lithium-ion diffusion rate is described in Example 11 below.

Without wishing to be bound by theory, it is believed that the enhanced Li-ion diffusion rate of the electrode 3000 can be explained based on the morphology of the composition 1000 and/or 2000 where particles of the crystalline metal oxide (e.g., SnO₂) 1200 are embedded in the organic compound (e.g., terephthalic acid) 1100. The electrode 3000 can exhibit both ionic conductivity and healing capability, due to the efficient formation of ion transport channels by the organic compound (e.g., terephthalic acid) 1100. The organic compound (e.g., terephthalic acid) 1100 matrix can effectively accommodate volume changes involved in the lithiation/dilithiation of the crystalline metal oxide (e.g., SnO₂) 1200 over prolonged cycling, promoted by the formation of hydrogen bonding between the two components.

Without wishing to be bound by theory, it is believed that the Li-ion diffusion rate increases in a cycled electrode containing the composition 1000 and/or 2000 relative to a non-cycled electrode containing the composition 1000 and/or 2000 as the volume changes involved in the cycling leads to the pulverization of the crystalline metal oxide (e.g., SnO₂) 1200 particles into finer particles, followed by the rearrangement of the fine particles in the organic compound 1100 matrix, and the formation of new hydrogen bonding between fine crystalline metal oxide (e.g., SnO₂) 1200 particles and organic compound (e.g., terephthalic acid) 1100. These interactions can lead to an increase of the surface area of the active material, a decrease in the lithium ion diffusion distances, and/or an enhancement of the electron and lithium ion transport on active materials/electrolyte interfacial area.

In some embodiments, a sodium ion (“Na-ion”) insertion into of the electrode 3000 occurs at a voltage of at least 0.1 (e.g., at least 0.11, at least 0.12, at least 0.13, at least 0.14, at least 0.15, at least 0.16, at least 0.17, at least 0.18, at least 0.19, at least 0.2, at least 0.25, at least 0.3 at least 0.35, at least 0.4, at least 0.45, at least 0.5, at least 0.55, at least 0.6, at least 0.65, at least 0.7, at least 0.75, at least 0.8, at least 0.85) V and/or at most 0.9 (e.g., at most 0.85, at most 0.8, at most 0.75, at most 0.7, at most 0.65, at most 0.6, at most 0.55, at most 0.5, at most 0.45, at most 0.4, at most 0.35, at most 0.3, at most 0.25, at most 0.2, at most 0.15) V. In some embodiments, a sodium ion (“Na-ion”) extraction out of the electrode 3000 occurs at a voltage of at least 0.3 (e.g., at least 0.4, at least 0.5, at least 0.6) V and/or at most 0.7 (e.g., at most 0.6, at most 0.5, at most 0.4) V.

In some embodiments, the binder includes PI. Without wishing to be bound by theory, it is believed that an electrode including a composition of the disclosure with SiNPs, PI, and with a heat treatment of the electrode can provide an electrode with enhanced electrochemical performance. The heat treatment can include heating to a temperature of at least 150 (e.g., at least 200, at least 250) ° C. and/or at most 400 (e.g., at most 350, at most 300, at most 250, at most 200) ° C. under flow of inert gas with H₂. Without wishing to be bound by theory, it is believed that hydrogen bonding between the PI and oxygen on the surfaces of the SiNPs can reduce (e.g., inhibit) disintegration of the electrode due to the expansion and contraction of the SiNPs. Without wishing to be bound by theory, it is believed that the formation of charge transfer complex structures within the PI chains during the heat-treatment process increases the toughness of the resultant electrode. In some embodiments, the enhanced electrochemical performance can include increased Li-ion storage capacity retention over several Li-ion insertion and extraction cycles relative to certain other electrode materials.

In some embodiments, heat-treatment of an electrode of the disclosure that includes Si (e.g., Si nanoparticles, a Si-containing composition of the disclosure) with PI as the binder (referred to herein as “Si@PI”) can result in a charge transfer complex (CTC) structure. Without wishing to be bound by theory, it is believed that such a CTC structure can improve the electrochemical performance of the electrode by forming a compact structure that reduces the charge transfer impedance. Also without wishing to be bound by theory, it is believed that this can substantially enhance the cycling performance of the silicon anode. For example, in some embodiments, electrodes containing Si with PI as the binder which are subjected to heat-treatment at 350° C. (Si@PI-350) exhibit a charge transfer impedance of 37.67Ω combined with a reversible Li⁺ storage capacity of 2334 mAh g⁻¹ recorded after 30 cycles at 200 mA g⁻¹, in comparison to the original (non-heat treated) Si@PI electrodes showing an enhanced impedance value of 130.4Ω and reduced capacity of 737 mAh g⁻¹. At a high current density of 2000 mA g⁻¹, the capacity of Si@PI-350 (1001 mAh g⁻¹) is substantially greater than Si@PI (455 mAh g⁻¹). This highlights the efficiency of the CTC structures formed during the thermal treatment. In some embodiments, the thermal treatment of Si@PI electrodes can significantly affect the Li-ion insertion/extraction cycling performance of the SiNPs.

In some embodiments, the electrodes of the disclosure include silicon particles and polymers containing imide monomers that are used as the binder. In some embodiments, the electrodes of the disclosure include SiNPs and PI. Such electrodes can be used, for example, as the anode of a metal-ion battery, such as a Li-ion battery, a Na-ion battery or a K-ion battery.

In some embodiments, the polymer (e.g., the PI) contains an imide group (—CO—N—CO—) on its main molecular chain. In some embodiments, the PI material is a thermoplastic polymer. In some embodiments the PI material is formed by the polycondensation and imidization of 1-(4-aminophenyl)-1,3,3-trimethyl-2H-inden-5-amine (DAPI) and benzophenone-3,3′,4,4′-tetra-carboxylic dianhydride (BTDA).

In some embodiments, the Si particles have sizes of at least 1 (e.g., at least 5, at least 10, at least 50, at least 100, at least 200, at least 400, at least 700, at least 1000, at least 3000, at least 5000) nm and/or at most 5000 (e.g., at most 3000, at most 1000, at most 700, at most 400, at most 200, at most 100, at most 50, at most 10, at most 5) nm. In some embodiments, the surface of silicon particles are at least partially oxidized to form SiOₓ (x=0.1-2.0). In some embodiments, silicon particles and polymers containing imide monomers are mixed with other additives such as conductive carbon. In some embodiments, silicon particles and polymers containing imide monomers are mixed with other additives such as conductive carbon and at least partially crystalline organic compound (e.g., a crystalline organic compound). In some embodiments, the at least partially crystalline organic compound is terephthalic acid. In some embodiment, the Si particles are embedded into the crystalline organic compound. In some embodiments, the mixture is heat-treated under a flow of inert gas at a target temperature for a specific period of time. In some embodiments, the target temperature is at least 140 (e.g., at least 150, at least 160, at least 180, at least 200, at least 230, at least 250, at least 280, at least 300, at least 318, at least 350, at least 400) ° C. and/or at most 400 (e.g., at most 350, at most 318, at most 300, at most 280, at most 250, at most 230, at most 200, at most 180, at most 160, at most 150) ° C. In some embodiments, the heating at target temperature is applied for at least 1 second (e.g., at least 5 seconds, at least 10 seconds, at least 10 minutes, at least 30 minutes, at least 1 hour, at least 5 hours) and/or at most 10 hours (e.g., at most 5 hours, at most 1 hour, at most 30 minutes, at most 10 minutes, at most 10 seconds, at most 5 seconds). In some embodiments, the heating atmosphere contains hydrogen with a volume percentage of at least 0.1 vol. % and/or at most 100.0 vol. %. In some embodiments, the heating atmosphere, in addition to hydrogen, contains argon, nitrogen and/or helium with a volume percentage of at least 0.1 vol. % and/or at most 99.9 vol. %.

Without wishing to be bound by theory, it is believed that the formation of hydrogen bonding between the polymer and the oxide phases present of the surfaces of Si particles, brought about by the heat-treatment process, can increase the stability of metal-ion insertion and extraction cycling processes in comparison with certain other electrodes. Additionally, also without wishing to be bound by theory, it is believed that the formation of charge transfer complexes within the polymer chains, brought about by the heat-treatment process, can increase the stability of metal-ion insertion and extraction cycling processes in comparison with certain other electrodes. Without wishing to be bound by theory, it is believed that the increase in cycling stability of the electrode is related to the crosslinking of the PI polymer.

In some embodiments, in the presence of the nanostructured organic compound, the PI reacts with the nanostructured organic compound during the heat-treatment to form a crystalline organic compound with a different XRD pattern than those of the PI and the nanostructured organic compound. In some embodiments, this reaction occurs at a temperature in the range of 250 to 400° C.

Without wishing to be bound by theory, in some embodiments, it is believed that the interaction between the polymer binder and the at least partially crystalline organic compound increases the toughness of the electrode in comparison with certain other electrodes.

In some embodiments, the electrode is formed from a mixture containing Si particles and the binder. In some embodiments, the Si particles are incorporated in a composition of the disclosure (e.g., the composition 2000). In some embodiments, the binder is a polymer containing imide monomers and the binder is at least 0.1 (e.g., at least 1, at least 5, at least 10, at least 15, at least 20, at least 25, at least 30, at least 35) wt. % and/or at most 40 (e.g., at most 35, at most 30, at most 25, at most 20, at most 15, at most 10, at most 5) wt. % of the mixture. In some embodiments, the silicon particles are at least 5 (e.g., at least 10, at least 15, at least 20, at least 25, at least 30, at least 35, at least 40, at least 45, at least 50, at least 55, at least 60, at least 65, at least 70, at least 75, at least 80, at least 85, at least 90) wt. % and/or at most 95 (e.g., at most 90, at most 85, at most 80, at most 75, at most 70, at most 65, at most 60, at most 55, at most 50, at most 45, at most 40, at most 35, at most 30, at most 25, at most 20, at most 15, at most 10) wt. % of the mixture. In some embodiments, the organic compound (e.g., terephthalic acid is at least 0.1 (e.g., at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90) wt. % and/or at most 95 (e.g., at most 90, at most 85, at most 80, at most 75, at most 70, at most 65, at most 60, at most 55, at most 50, at most 45, at most 40, at most 35, at most 30, at most 25, at most 20, at most 15, at most 10, at most 5, at most 1) wt. % of the mixture.

In some embodiments, the PI is insoluble in polar solvents such as NMP after the heat-treatment. In some embodiments the electrodes are fabricated by mixing materials containing silicon particles, PI and conductive carbon and the mixture is heat-treated at temperatures of at least 200 (e.g., at least 250, at least 300, at least 350) ° C. and/or at most 400 (e.g., at most 350, at most 300, at most 250) ° C. In some embodiments the electrodes provide a Li-ion storage charge capacity of at least at least 700 (e.g., at most 1000, at most 1500, at most 2000, at most 2500) mAh g⁻¹ and/or at most 3000 (e.g., at most 2500, at most 2000, at most 1500, at most 1000) mAh g⁻¹ after 100 Li-ion insertion and extraction cycles. In some embodiments, the Li-ion diffusion impedance (Rₛ) of the electrode obtained after the heat-treatment process is greater than that of the initial electrode before the heat-treatment process. In some embodiment, the mixture is heat-treated at 350° C., and the charge transfer resistance of the electrode reduces from 130.4Ω to 37.7Ω. In some embodiments, the electrode is fabricated by the heat-treatment of a mixture containing a metal-ion active material such as Si particles and a polymer containing imide monomers. The mixture is heat-treated at a maximum temperature in the range of 150-400° C. for is to 10 h. The metal-ion diffusion impedance (Rs) of the electrode made of the composition is at least 10 (e.g., at least 20, at least 30, at least 40, at least 50) Ω and/or at most 60 (e.g., at most 50, at most 40, at most 30, at most 20).

### Water Purification

A composition of the disclosure (e.g., the nanostructured organic compound, the composition 1000, or the composition 2000) can be used for water purification (see Example 19, for example). In some embodiments, the composition includes SnO and/or Sn. The SnO and/or Sn may be crystalline. A composition of the disclosure can reduce a concentration of a contaminant (e.g., an organic contaminant) in an aqueous solution. Without wishing to be bound by theory, it is believed that contaminants can be adsorbed onto a surface of the composition. Additionally, without wishing to be bound by theory, it is believed that under visible light exposure (e.g., 400-650 nm excitation) the composition can photocatalytically degrade contaminants. Examples of the contaminants include a hydrocarbon such as azo dye such as methyl yellow, methyl orange, methyl red, congo red, alizarin yellow, methyl blue, methylene blue, and rhodamine; and a xanthate-based compound, such as potassium ethyl xanthate, sodium isopropyl xanthate, sodium isobutyl xanthate, sodium butyl xanthate and butyl xanthate. The hydrocarbon can include a component of a produced oil or gas, a component of crude oil, an alkane (e.g., methane, ethane, propane, butane, pentane, hexane), an alkene, an alkyne, a halogenated compound, and/or an aromatic compound (e.g., benzene, toluene, xylene).

In certain embodiments, a composition of the disclosure has an adsorption capacity, as defined in equation (12) in Example 19, of at least 5 (e.g., at least 10, at least 15, at least 20, at least 25) mg/g and/or at most 30 (e.g., at most 25, at most 20, at most 15, at most 10) mg/g. In certain embodiments, a composition of the disclosure has an organic compound removal performance, as defined in equation (13) of Example 19 of at least 1 (e.g., at least 2, at least 3, at least 4, at least 5, at least 6, at least 7, at least 8, at least 9) mg/(g×h) and/or at most 10 (e.g, at most 9, at most 8, at most 7, at most 6, at most 5, at most 4, at most 3, at most 2) mg/(g×h) under light irradiation.

### Depolymerization of Polymers and Synthesis of Compounds

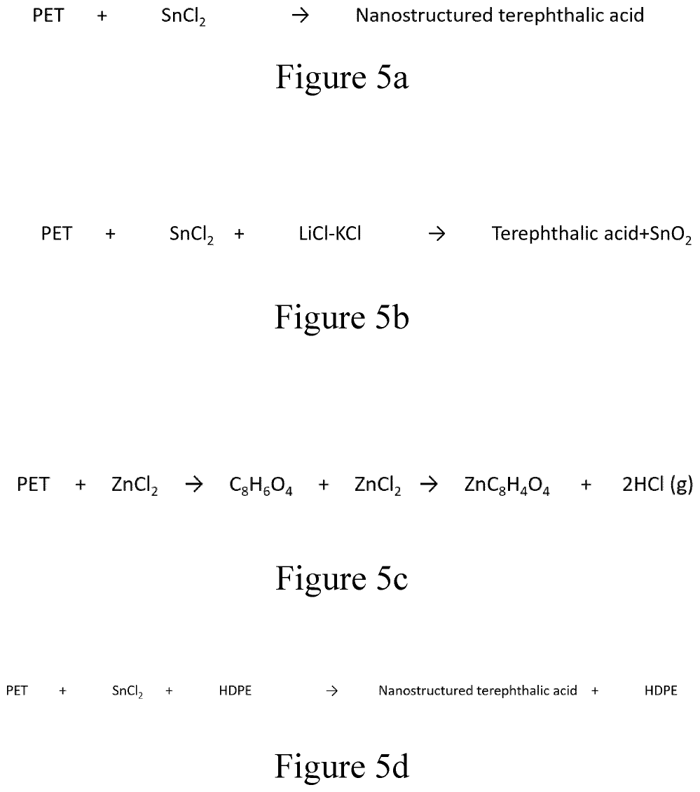

FIG. 5a shows a reaction scheme for the synthesis of nanostructured terephthalic acid. A mixture containing the reactants polyethylene terephthalate (PET) and SnCl₂ is heated to form nanostructured terephthalic acid. The polymer (PET) is depolymerized to form terephthalic acid. In some embodiments, the reactants further include a salt and heating the mixture makes the salt a molten salt.

FIG. 5b shows a reaction scheme for the synthesis of a composition 1000 containing terephthalic acid and SnO₂. A mixture containing the reactants polyethylene terephthalate (PET), SnCl₂ and KCl—LiCl is heated to form the composition 1000 (terephthalic acid+SnO₂). The polymer (PET) is depolymerized to form terephthalic acid, which forms the organic compound 1100 in the composition 1000.

Without wishing to be bound by theory, it is believed that the PET undergoes depolymerization to form terephthalic acid due to the presence of SnCl₂, which acts as a depolymerization agent. Without wishing to be bound by theory, it is believed that in the solid phase SnCl₂ exists as polymeric chains. Upon melting, SnCl₂ maintains its polymeric structure (SnCl₂)ₙ with three-coordinated Sn²⁺. Further increasing the temperature can reduce the degree of polymerization, thereby reducing the viscosity. Without wishing to be bound by theory, it is believed that PET and SnCl₂ melt at around 250° C. to form two polymeric melts. Increasing the temperature to 350° C. can create free Sn²⁺ and Cl⁻ that can break the chains of PET to form terephthalic acid.

Generally, the reactants include a polymer (e.g., PET) and a depolymerization agent (e.g., SnCl₂). In some embodiments, the reactants further include a salt (e.g., LiCl—KCl). Without wishing to be bound by theory, heating the mixture makes the salt a molten salt.

In some embodiments, the depolymerization of the polymer can create a first organic compound (e.g., terephthalic acid) and a second organic compound (e.g., ethylene glycol) that has a lower boiling point from the first organic compound. Without wishing to be bound by theory, if the first organic compound is incorporated into a product of the disclosure (e.g., the nanostructured organic compound, the organic compound 1100 in the composition 1000 or 2000), the second organic compound can be separated relatively easily, such as by evaporating the second organic compound. In some embodiments, the second organic compound is evaporated upon its formation. In such embodiments, the second organic compound can be condensed and collected as a liquid.

Without wishing to be bound by theory, it is believed that water, such as water associated with the depolymerization agent (e.g., SnCl₂, ZnCl₂), plays a role in the depolymerization of the polymer, as shown in reaction (3)

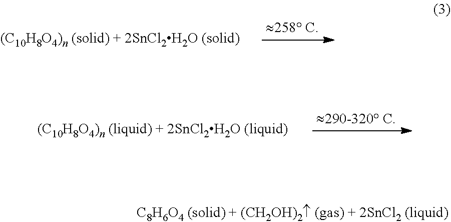

The hydrated SnCl₂ melts at around 258° C. and the SnCl₂ retains at least a portion (e.g., the majority) of its water content (see Example 22). In certain embodiments, at least a portion (e.g., all) of the depolymerization agent is hydrated. In certain embodiments, at least a portion (e.g., all) of the depolymerization agent maintains water at least until the depolymerization agent acts to depolymerize the polymer. In certain embodiments, the depolymerization agent includes at least 0.1 (e.g., at least 0.2, at least 0.3, at least 0.4, at least 0.5, at least 0.6, at least 0.7, at least 0.8, at least 0.9, at least 1, at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19) wt. % water and/or at most 20 (e.g., at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, at most 1.5, at most 1, at most 0.9, at most 0.8, at most 0.7, at most 0.6, at most 0.5, at most 0.4, at most 0.3, at most 0.2) wt. % water. Without wishing to be bound by theory, the hydration of the reactants (e.g., the depolymerization agent) may be due to moisture from the atmosphere. In certain embodiments, at least a portion (e.g., all) of the depolymerization agent absorbs moisture from environment, for example the surrounding atmosphere.

In certain embodiments, the reactants contain at least 1 (e.g., at least 2, at least 5, at least 10, at least 15, at least 20, at least 25, at least 30, at least 35, at least 40, at least 45, at least 50) wt. % and/or at most 95 (e.g., at most 90, at least 85, at least 80, at least 75, at least 70, at least 65, at least 60, at least 55, at least 50) wt. % of the depolymerization agent.

In some embodiments, the depolymerization agent is an inorganic salt. Examples of inorganic salts include tin(II) chloride (SnCl₂), zinc chloride (ZnCl₂), calcium chloride (CaCl₂)), lead chloride (PbCl₂), sodium chloride (NaCl), potassium chloride (KCl) and iron chloride (FeCl₂).

In certain embodiments, the inorganic salt contains a metal of the crystalline metal oxide 1200. In certain embodiments, the inorganic salt undergoes an oxidation during the heating. For example, SnCl₂ undergoes oxidation from oxygen present in the atmosphere to form SnO₂.

Without wishing to be bound by theory, the phase transition of SnCl₂ into SnO₂ nanoparticles is:

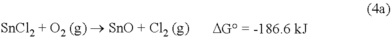

where O₂ is consumed to form SnO₂ and Cl₂ gas is released.

Generally, the depolymerization agent has a melting point relatively close to the melting point of the polymeric material. In certain embodiments, the different in melting temperatures of the polymer and the depolymerization agent is less than 100 (e.g., less than 95, less than 90, less than 85, less than 80, less than 75, less than 70, less than 65, less than 60, less than 55, less than 50) ° C.

In general, the polymer (e.g., PET) depolymerizes to form the organic compound (e.g., terephthalic acid). In some embodiments, the reactants contain at least 1 (e.g., at least 2, at least 5, at least 10, at least 15, at least 20, at least 25, at least 30, at least 35, at least 40, at least 45, at least 50) wt. % and/or at most 99 (e.g., at most 95, at most 90, at least 85, at least 80, at least 75, at least 70, at least 65, at least 60, at least 55, at least 50) wt. % of the polymer. Examples of the polymer include polyethylene terephthalate (PET), poly(acrylonitrile), poly(6-aminocaproic acid), poly(caprolactam), Nylon, poly(etheretherketone) (PEEK), Poly(ethylene) (PE), poly(hexamethylene adipamide), poly(methyl methacrylate), poly(methylene oxide), poly(4-methylpentene), poly(propylene), poly(styrene), poly(trans-1,4-butadiene), poly(vinyl alcohol), poly(vinyl chloride) (PVC), poly(vinyl fluoride), poly(vinylidene chloride), and poly(vinylidene fluoride). In some embodiments, the polymer is derived from waste plastic.

Without wising to be bound by theory, it is believed that the organic compound (e.g., terephthalic acid) can undergo sublimation and/or decomposition at relatively high temperatures (e.g., greater than 500° C., greater than 600° C., greater than 700° C., greater than 800° C.). Without wishing to be bound by theory, it is believed that the phase transitions are:

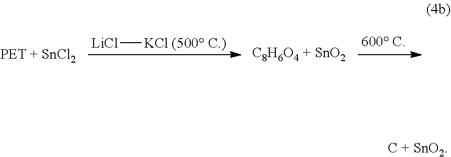

In some embodiments, the salt contains a chloride salt (e.g., LiCl and/or KCl). In some embodiments, the salt contains at least 40 (e.g., at least 45, at least 50, at least 55, at least 60) wt. % and/or at most 80 (e.g., 75, at most 70, at most 65, at most 60, at most 55, at most 50) wt. % LiCl. In some embodiments, the salt contains at least 20 (e.g., at least 25, at least 30, at least 35, at least 40, at least 45, at least 50) wt. % and/or at most 70 (e.g., at most 65, at most 60, at most 55, at most 50, at most 45, at most 40) wt. % KCl. In some embodiments, the salt contains at least 40 (e.g., at least 45, at least 50, at least 55, at least 60) wt. % and/or at most 80 (e.g., 75, at most 70, at most 65, at most 60, at most 55, at most 50) wt. % LiCl and/or at least 20 (e.g., at least 25, at least 30, at least 35, at least 40, at least 45, at least 50) wt. % and/or at most 70 (e.g., at most 65, at most 60, at most 55, at most 50, at most 45, at most 40) wt. % KCl. In some embodiments, the salt contains a eutectic mixture of LiCl—KCl. In some embodiments, the salt has a melting point of at least 250 (e.g., at least 300, at least 320) ° C. and/or at most 700 (e.g., at most 650, at most 600, at most 550, at most 500) ° C.

Without wishing to be bound by theory, it is believed that the salt (e.g., a eutectic mixture of KCl—LiCl) provides an ionic environment to enhance the formation of at least partially crystalline (e.g., crystallized) organic compound (e.g., terephthalic acid) monomers, plays a role in the formation of porosity within the resulting composition and supports the phase transition of the depolymerization agent to the crystalline metal oxide (e.g., molten SnCl₂ to SnO₂ particles) leading to the formation of at least partially crystalline organic compound (e.g., terephthalic acid) with crystalline metal oxide (e.g., SnO₂) distributed within.

Generally, the reactions of the disclosure (e.g., the reactions depicted in FIGS. 5a-c) can be performed in any suitable atmosphere. In some embodiments, a reaction of the disclosure (e.g., the reactions in FIGS. 5a-c) is performed in an atmosphere containing oxygen (e.g., air). In some embodiments, the atmosphere contains at least 1 (e.g., at least 2, at least 5, at least 10, at least 15, at least 20, at least 21) percent by volume oxygen. In some embodiments, a reaction of the disclosure (e.g., the reactions depicted in FIGS. 5a-c) is performed under an inert atmosphere (e.g., argon atmosphere, nitrogen gas atmosphere) and/or hydrogen gas (as described in Examples 14, 17 and 18 below). In some embodiments, a reaction of the disclosure (e.g., the reactions in FIGS. 5a-c) is performed under an atmosphere containing argon and at least 1 (e.g., at least 2, at least 4, at least 5, at least 10, a least 15, at least 20, at least 25, at least 30, at least 35, at least 40, at least 45, at least 50, at least 55, at least 60, at least 65, at least 70, at least 75, at least 80, at least 85, at least 90, at least 95) vol. % and/or at most 99 (e.g., at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 5, at most 4, at most 2) vol. % hydrogen gas.

Without wishing to be bound by theory, it is believed that performing the reaction under an inert atmosphere and/or a hydrogen gas can reduce (e.g., prevent) oxidation of SnCl₂ into SnO₂ by oxygen present in the atmosphere, thereby forming a composition without SnO₂, or with a reduced amount of SnO₂ relative to a reaction performed in the presence of oxygen, or an oxide with reduced amount of oxygen. In such embodiments, in addition to or instead of SnO₂, the composition can contain SnO and/or Sn. Without wishing to be bound by theory, it is believed that performing the reaction under an inert atmosphere and/or hydrogen gas can alter the organic compounds formed from the depolymerization of the polymer. In general, heating is performed to achieve a temperature greater than a melting point of the polymer, a melting point of the salt and a melting point of the depolymerization agent and/or at a temperature lower than a carbonization temperature of the polymer and a decomposition temperature of the organic compound. In certain embodiments, the mixture is heated to a maximum temperature of at least 250 (e.g., at least 300, at least 310, at least 350, at least 400, at least 450, at least 500) ° C. and/or at most 600 (e.g., at most 550, at most 500, at most 450, at most 400, at most 350, at most 310, at most 300) ° C. In certain embodiments, the mixture is held at the maximum temperature for at least 0.01 (e.g., at least 0.017, at least 0.1, at least 1, at least 2, at least 5, at least 10, at least 20, at least 30, at least 40, at least 50, at least 60) minutes and/or at most 120 (e.g., at most 110, at most 100, at most 90, at most 80, at most 70, at most 60, at most 50, at most 40, at most 30, at most 20, at most 10) minutes. In certain embodiments, the mixture is held at the maximum temperature for 1 second. In certain embodiments, the mixture is heated at a rate of at least 1 (e.g., at least 2, at least 3, at least 4, at least 5, at least 10, at least 15, at least 20, at least 30, at least 40, at least 50) ° C. min⁻¹ and/or at most 100 (e.g., at most 90, at most 80, at most 70, at most 60, at most 50, at most 40, at most 30, at most 20, at most 10, at most 5) ° C. min⁻¹.

In some embodiments, a reaction of the disclosure (e.g., the reactions in FIGS. 5a-c) can include cooling the mixture after heating the mixture. In some embodiments, the cooling is performed under the same atmosphere as the heating (see discussion above).

Generally, the reactions of the disclosure (e.g., the reactions depicted in FIGS. 5a-c) can be performed in any suitable pressure. In certain embodiments, a reaction of the disclosure (e.g., the reactions depicted in FIGS. 5a-c) is performed at a pressure of at least 0.01 (e.g., at least 0.05, at least 0.1, at least 0.2, at least 0.25, at least 0.3, at least 0.4, at least 0.5, at least 0.6, at least 0.7, at least 0.75, at least 0.8, at least 0.9, at least 1, at least 1.5, at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95) atm and/or at most 100 (e.g., at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 5, at most 2, at most 1, at most 0.9, at most 0.8, at most 0.75, at most 0.7, at most 0.6, at most 0.5, at most 0.4, at most 0.3, at most 0.25, at most 0.2, at most 0.1, at most 0.05) atm. In certain embodiments, a reaction of the disclosure (e.g., the reactions depicted in FIGS. 5a-c) is performed at atmospheric pressure.

In some embodiments, the reactants and/or the composition is (are) contacted with a solvent during the synthesis. In some embodiments, the solvent is an aqueous solution (e.g., an alkali aqueous solution, an acidic aqueous solution) and/or contains a polar organic liquid. In some embodiments, the polar organic liquid is an alcohol (e.g., methanol, ethanol, propanol, butanol).

In some embodiments, the solvent has a pH of at least 0 (e.g., at least 0.1, at least 1, at least 2, at least 3, at least 4, at least 5, at least 6) and/or at most 7 (e.g., at most 6, at most 5, at most 4, at most 3, at most 2, at most 1). In some embodiments, the solvent contains hydrochloric acid, sulfuric acid, nitric acid and/or phosphoric acid. In some embodiments, the solvent contains an acid at a concentration at least 1 (e.g., a t least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90) percent by volume (vol. %) and/or at most 98 (e.g., at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 5) vol. %.

In some embodiments, the solvent has a pH of at least 7 (e.g., at least 8, at least 9, at least 10, at least 11, at least 12, at least 13) and/or at most 14 (e.g., at most 13, at most 12, at most 11, at most 10, at most 9, at most 8). In some embodiments, the solvent contains a hydroxide (e.g., sodium hydroxide, potassium hydroxide). In some embodiments, the solvent contains a base acid at a concentration at least 1 (e.g., a t least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90) vol. % and/or at most 98 (e.g., at most 95, at most 90, at most 85, at most 80, at most 75, at most 70, at most 65, at most 60, at most 55, at most 50, at most 45, at most 40, at most 35, at most 30, at most 25, at most 20, at most 15, at most 10, at most 5) vol. %.

In certain embodiments, a composition of the disclosure (e.g., the nanostructured organic compound, the composition 1000, the composition 2000) includes the depolymerization agent and contacting the composition with the solvent (e.g., a leaching agent such as acidic water, HCl, H₂SO₄ or HNO₃) removes at least a portion of the depolymerization agent. In certain embodiments, composition of the disclosure (e.g., the nanostructured organic compound, the composition 1000, the composition 2000) includes the depolymerization agent and contacting the composition with the solvent hydrolyzes at least a portion of the depolymerization agent.

In some embodiments, the depolymerization agent is SnCl₂ and the hydrolysis of SnCl₂ forms a second material with tin, chlorine, hydrogen and oxygen, where the second material is dispersed within the organic compound of the composition. In some embodiments, the second material is a tin oxide chloride hydroxide. In some embodiments, the second material stoichiometry of Sn₂₁Cl₁₆(OH)₁₄O₆. In some embodiments, the second material is at least partially crystalline (e.g., crystalline).

In some embodiments, contacting the composition with the solvent (e.g., a leaching agent such as acidic water, hydrochloric acid, sulfuric acid or nitric acid) removes a Sn-containing material (e.g., SnO₂, SnO, Sn, SnCl₂) from the composition. In some embodiments, a composition containing Si, SnO₂, and terephthalic acid (e.g., as described in Example 15), can be contacted with a solvent (e.g., sulfuric acid, hydrochloric acid) to remove the SnO₂. Without wishing to be bound by theory, it is believed that this can avoid the hydrolysis reaction (1) (see discussion above).

In embodiments where a solvent is used, the methods can further include separating the composition from the solvent and/or drying the composition. Methods of separation are known in the art and include vacuum filtration and centrifugation. In some embodiments, the composition is dried under air, an inert atmosphere or vacuum. In certain embodiments, the composition is dried at a temperature of at least −196 (e.g., −100, −50, 0, 20) ° C. and/or at most 100 (e.g., at most 50, at most 20, at most 0) ° C.

As discussed previously, in some embodiments, the composition contains a silicon-containing material and/or graphene nanosheets. In some embodiments, the mixture contains a precursor of a silicon-containing material, and the resulting composition contains the silicon-containing material. In some embodiments, the mixture contains graphene nanosheets and the resulting composition contains graphene nanosheets. In some embodiments, the solvent contains a precursor of a silicon-containing material, and the resulting composition contains the silicon-containing material. In some embodiments, the solvent contains graphene nanosheets, and the resulting composition contains the graphene nanosheets.

In certain embodiments, the reactants contain at least 0.1 (e.g., at least 0.2, at least 0.5, at least 1, at least 2, at least 5, at least 10, at least 15, at least 20, at least 25, at least 30, at least 35, at least 40, at least 45, at least 50) wt. % and/or at most 98 (e.g., at most 95, at most 90, at most 85, at most 80, at most 75, at most 70, at most 65, at most 60, at most 55, at most 50) wt. % of the silicon-containing precursor. Examples of precursors of the silicon-containing material include elemental silicon, Ca₂Si, Ca₅Si₃, CaSi, Ca₃Si₄, CaSi₂ and Mg₂Si. In certain embodiments, the precursor of the silicon-containing material contains nanoparticles. In certain embodiments, the precursor of the silicon-containing material have a particle size of at least 1 (e.g., at least 2, at least 3, at least 4, at least 5) nm and/or at most 1000 (e.g., at most 500, at most 200, at most 100, at most 50, at most 20, at most 10, at most 5) nm.

In some embodiments, the precursor of the silicon-containing material is balled-milled. In some embodiments, the precursor of the silicon-containing material is ball-milled with a solvent. In some embodiments, the solvent is n-hexanes. Without wishing to be bound by theory, it is believed that the solvent (e.g., n-hexane) prevents the oxidation of silicon particles during the ball-milling process. Furthermore, without wishing to be bound by theory, it is believed that the solvent (e.g., n-hexane) can functionalize the silicon surfaces during the mechanical milling, which may provide desirable properties to the final composition.

In certain embodiments, the reactants contain at least 0.1 (e.g., at least 0.2, at least 0.5, at least 1, at least 2, at least 5, at least 10, at least 15, at least 20, at least 25, at least 30, at least 35, at least 40, at least 45, at least 50) wt. % and/or at most 80 (e.g., at most 75, at most 70, at most 65, at most 60, at most 55, at most 50, at most 45, at most 40, at most 35, at most 30, at most 25, at most 20, at most 15, at most 10, at most 5) wt. % of the graphene nanosheets. In certain embodiments, the graphene nanosheets contain flakes having a flake size of at least 1 (e.g., at least 2, at least 5, at least 10, at least 20, at least 50, at least 100) nm and/or at most 5 (e.g., at most 4, at most 3, at most 2, at most 1) μm. In certain embodiments, the graphene nanosheets have at least 1 (e.g., at least 2, at least 3, at least 4, at least 5) layers and/or at most 100 (e.g., at most 90, at most 80, at most 70, at most 60, at most 50, at most 40, at most 30, at most 20, at most 10) layers. In certain embodiments, the graphene nanosheets have a carbon purity of at least 90 (e.g., at least 91, at least 92, at least 93, at least 94, at least 95) %. In certain embodiments, the graphene nanosheets contain functional groups (e.g., hydroxyl group, a carbonyl group, a carboxyl group and/or an amino group) on their surface.

In some embodiments, the graphene nanosheets are produced through a cathodic electrochemical exfoliation of graphite electrodes. In some embodiments, cathodic electrochemical exfoliation is performed in a molten salt (e.g., lithium chloride and/or sodium chloride). In some embodiments, the cathodic electrochemical exfoliation is performed at a temperature of at least 500 (e.g., at least 600, at least 700, at least 800) ° C. and/or at most 900 (e.g., at most 800, at most 700, at most 600) ° C.

In certain embodiments, graphene nanosheets can be introduced into a composition of the disclosure by forming a suspension with the composition and graphene nanosheets and sonicating the suspension to form a product include the composition and the graphene nanosheets. In certain embodiments, the suspension includes an acid. In certain embodiments, the product contains at least 50 (e.g., at least 55, at least 60, at least 65, at least 70, at least 75, at least 80, at least 85, at least 90, at least 95, at least 99, at least 99.5) wt. % and/or at most 99.9 (e.g., at most 99.5, at most 99, at most 90, at most 85, at most 80, at most 75, at most 70, at most 65, at most 60, at most 55) wt. % of the composition. In certain embodiments, the product contains at least 0.1 (e.g., at least 0.2, at least 0.5, at least 1, at least 2, at least 5, at least 10, at least 15, at least 20, at least 25, at least 30, at least 35, at least 40, at least 45) wt. % and/or at most 50 (e.g., at most 45, at most 40, at most 35, at most 30, at most 25, at most 20, at most 15, at most 10, at most 5, at most 2, at most 1) wt. % of the graphene nanosheets.

FIG. 5c shows a reaction scheme for the synthesis of a composition of the disclosure that includes a metal-organic framework (see discussion above). A mixture containing the reactants polyethylene terephthalate (PET) and ZnCl₂ is heated to form terephthalic acid (C₈H₆O₄) by depolymerizing PET. The terephthalic acid (C₈H₆O₄) and ZnCl₂ form zinc terephthalate (ZnC₈H₄O₄) and HCl. The HCl can be removed from the zinc terephthalate (ZnC₈H₄O₄). The zinc terephthalate (ZnC₈H₄O₄) formed by the reaction shown in FIG. 5c is a zinc-based metal-organic framework. The zinc terephthalate (ZnC₈H₄O₄) is embedded in an organic compound (see discussion above). In some embodiments, the zinc terephthalate is hydrated. In some embodiments, the zinc terephthalate has the formula ZnC₈H₄O₄·nH₂O and n is 0 to 5. In some embodiments, n is 0, 1, 2 or 3.5.

In some embodiments, zinc hydroxide chloride is used as the depolymerization agent. Without wishing to be bound by theory, zinc hydroxide chloride can decompose to form ZnCl₂ upon heating.

In certain embodiments, a composition of the disclosure includes a metal-organic framework that includes hydrated water and the composition is heated to a second temperature, which removes at least a portion of the hydrated water from the metal-organic framework. In certain embodiments, the metal-organic framework has a first crystal structure prior to the heating to the second temperature and the metal-organic framework has a second crystal structure different from the first crystal structure after heating to the second temperature (see Example 32). In certain embodiments, the second temperature is at least 50 (e.g., at least 100, at least 150, at least 200, at least 250, at least 300, at least 350) ° C. and/or at most 400 (e.g., at most 350, at most 300, at most 250, at most 200, at most 150, at most 100) ° C. In certain embodiments, the composition is held at the second temperature for at least 1 millisecond (e.g., at least 0.1 seconds, at least 1 second, at least 10 seconds, at least 1 minute, at least 10 minutes, at least 1 hour) and/or at most 10 hours (e.g., at most 1 hour, at most 10 minutes, at most 1 minute, at most 10 seconds, at most 1 second).

In some embodiments, the reactions shown in FIGS. 5a and 5b can include removing at least a portion of the composition (e.g., nanostructured terephthalic acid, terephthalic acid+SnO₂) and adding additional polymer (e.g. PET).

In some embodiments, the methods of the disclosure further include the preparation of Na₂TP (Na₂C₈H₄O₄), Li₂TP (Li₂C₈H₄O₄), K₂TP (K₂C₈H₄O₄) or ZnTP (ZnC₈H₆O₄) from a compound of the disclosure. For example, Na₂TP can be prepared using acid-base reaction:

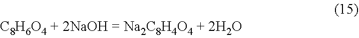

Without wishing to be bound by theory, it is believed that the compounds of the disclosure undergo the reaction depicted above with faster kinetics relative to certain other organic compounds (e.g., commercial terephthalic acid that is not nanostructured and/or nanocrystalline). The greater reaction kinetics in the nanostructured organic compound (e.g., terephthalic acid) may be related to its nanoscale size and nanocrystalline structure, allowing the material to react at greater kinetics in comparison with certain other organic compounds (e.g., commercial terephthalic acid that is not nanostructured and/or nanocrystalline). The rapid preparation of functional materials, such as Na₂TP, can reduce the cost of preparation, thereby promoting their use. For example, Na₂TP can be applied in the anode of Na-ion batteries.

Without wishing to be bound by theory, compounds such as Li₂TP (Li₂C₈H₄O₄), K₂TP (K₂C₈H₄O₄) and ZnTP (ZnC₈H₆O₄) could be produced by treating the nanostructured organic compound (e.g., nanostructured terephthalic acid) with appropriate solutions such as solutions of LiOH, KOH and Zn(OH)₂, respectively, at relatively short reaction times. Such compounds can be utilized as the electrodes of metal-ion batteries, such as Li-ion, K-ion and Zn-ion batteries, respectively.

In some embodiments, the reaction (15) can be performed in at most 18 (e.g., at most 16, at most 14, at most 12, at most 10, at most 8, at most 6, at most 5, at most 4, at most 3, at most 2, at most 1) hours.

### Separation of Plastics

FIG. 5d shows a reaction scheme. A mixture containing the reactants polyethylene terephthalate (PET), SnCl₂ and HDPE is heated to form nanostructured terephthalic acid. The polymer (PET) is depolymerized to form terephthalic acid whereas the HDPE does not undergo depolymerization. The HDPE can melt and sink to the bottom of the container, allowing relatively easy separation of the HDPE from the nanostructured terephthalic acid. Without wishing to be bound by theory, it is believed that the crystal structure of HDPE is not impacted by heating with a depolymerization agent (e.g., SnCl₂) (see Example 34).

While the reaction depicted in FIG. 5d shows SnCl₂ as the depolymerization agent, any depolymerization agent of the disclosure can be used. Similarly, while the reaction depicted in FIG. 5d shows the formation of nanostructured terephthalic acid, any composition of the disclosure can be formed using the appropriate reagents and reaction conditions.

Without wishing to be bound by theory, crystalline polymers are resistant to depolymerization due to their highly ordered structure and strong intermolecular forces whereas semi-amorphous or amorphous polymers can be more effectively depolymerized due to their less ordered structure. Additionally, polymers with a relatively high thermal stability, such as polyimides, may not be efficiently depolymerized.

In addition to PET, examples of the depolymerizable polymer include polystyrene, polyvinyl chloride, nylon, a polyurethane, a phenolic resin and an epoxy resin.

In addition to HDPE, examples of the non-depolymerizable (e.g., highly crystalline) polymer include a polyethylene and a polypropylene.

### EXAMPLES

### Example 1—Synthesis

Polyethylene terephthalate (PET) was cut into approximately 10×5 mm pieces using scissors. 20 g of PET pieces were placed in an alumina crucible with an internal diameter of approximately 5 mm and a height of approximately 100 mm. 10.10 g SnCl₂ (99.9%, Aladdin), 27.54 g KCl (99.9%, Aladdin) and 23.06 g LiCl (98%, Aladdin) were added to the crucible. The amounts of LiCl and KCl provided an eutectic composition (KCl: 54.8 wt. %-45.2 wt. % LiCl) with the melting point of about 360° C. The mixture was heated from room temperature to target temperatures of 500, 600, 700 and 800° C. (PDN-500, -600, -700, and -800, respectively) at a heating rate of 5° C. min⁻¹ in a vertical furnace equipped with an alumina tube. The dwell time at the maximum temperature was 20 minutes. The furnace was cooled with a rate of about 5° C. min⁻¹ to room temperature. The samples were washed with deionized water, vacuum filtered and dried at about 100° C. for 2 hours.

### Example 2—X-Ray Diffraction Measurements

Samples were measured on a powder diffractometer (Panalytical X'pert Pro) with Cu Kα radiation (λ=0.1542 nm) from 10 to 90° (2θ).

PET was heat treated with SnCl₂ at 300 and 350° C. for 20 minutes followed by washing and filtration of the sample.

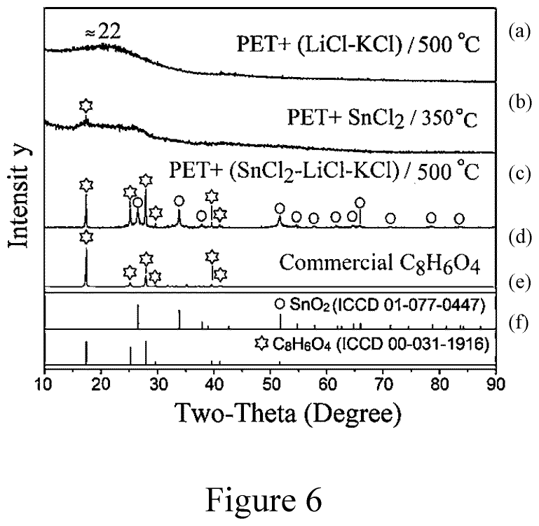

FIGS. 6a-c shows the X-ray diffraction pattern of PET heat treated with LiCl—KCl (PET+(LiCl—KCl)/500° C.), PET heat treated with SnCl₂ heated to 350° C. (PET+SnCl₂/350° C.), and PDN-500 (PET heat treated with SnCl₂ and LiCl—KCl with a target temperature of 500° C. as described in Example 1) (PET+(SnCl₂—LiCL-KCl)/500° C.), respectively. The XRD pattern for commercially available terephthalic acid (Commercial C₈H₆O₄), and standard diffraction patterns of SnO₂ and terephthalic acid are also shown in FIGS. 6d-f, respectively.

The XRD pattern of (PET+SnCl₂/350° C.) suggested the formation of terephthalic acid.

The XRD pattern of the PET heat treated with SnCl₂ at 350° C. shows evidence of the formation of terephthalic acid.

For PDN-500 (PET+(SnCl₂—LiCL-KCl)/500° C.)) diffraction peaks appeared in the two-theta values of around 26.60°, 33.90°, 37.97°, 39.00°, 51.81°, 54.79°, 57.87°, 61.92°, 64.79°, 66.01°, 71.33°, 78.76°, 81.19°, 83.78°, and 87.29° and can be indexed to the (110), (101), (200), (111), (211), (220), (002), (310), (112), (301), (202), (321), (400), (222), and (330) diffraction planes of the tetragonal SnO₂ (JCPDS≠01-070-4177), respectively. In addition to SnO₂, the diffraction pattern confirmed the presence of terephthalic acid (C₈H₆O₄, JCPDS≠031-1916) with anorthic crystalline structure. The peaks observed at 2θ=17.41°, 25.21°, 27.950 could be indexed to the (110), (0-10) and (200) diffraction planes of terephthalic acid. For comparison, the XRD pattern of commercially available terephthalic acid (C₈H₆O₄, Shanghai Macklin, 400-623-8666, 99%) is also shown in FIG. 6, confirming the formation of terephthalic acid by the molten salt process.

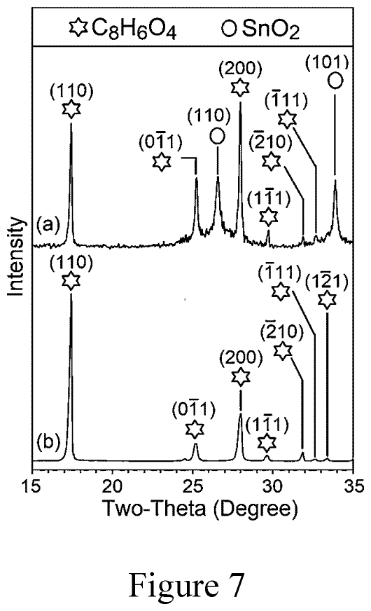

FIG. 7 shows the XRD patterns of (a) PDN-500 (PET heat treated with SnCl₂ and LiCl—KCl with a target temperature of 500° C. as described in Example 1 (PET+(SnCl₂—LiCL-KCl)/500° C.)) and (b) commercially available micrometer-sized terephthalic acid (C₈H₆O₄, Shanghai Macklin, 400-623-8666, 99%).

FIG. 7 further confirms the formation of terephthalic acid by the method described in Example 1.

### Example 3—Temperature Dependence

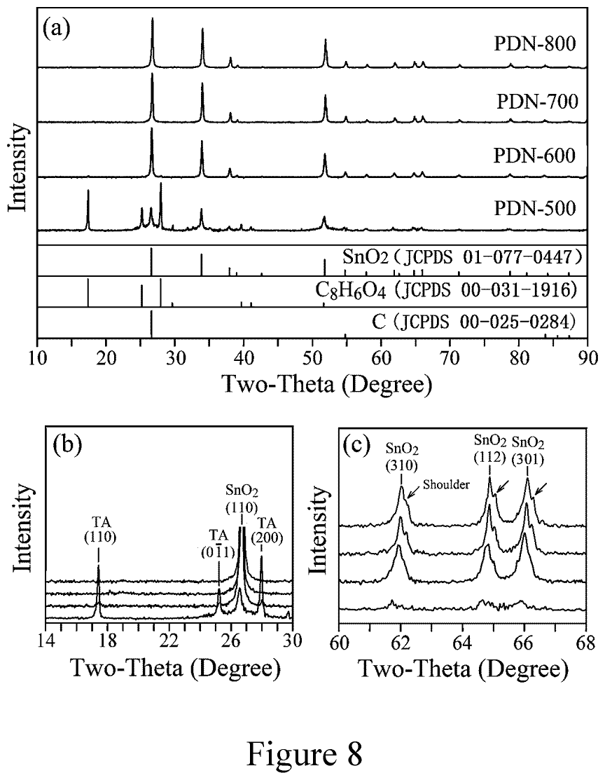

FIG. 8a shows the X-ray diffraction patterns of PDN-500, -600, -700, and -800 (the compounds synthesized from PET, SnCl₂, and LiCl—KCl, at target temperatures of 500, 600, 700 and 800° C. as described in Example 1) in the two-theta (2θ) range 10-90°. FIG. 8b shows high resolution XRD patterns of the samples in the two-theta range 14-30°. FIG. 8c shows high resolution XRD patterns of the samples in the two-theta range 60-68°. FIG. 8a additionally contains JCPDS reference patters.

FIGS. 8a and 8b show that X-ray peaks correspond to SnO₂ can be observed in all samples. However, peaks corresponding to terephthalic acid cannot be observed in the samples prepared at a target temperature of 600, 700 and 800° C.

FIG. 8a may further suggest the presence of graphitic carbon with the hexagonal crystalline structure (JCPDS≠00-025-0284). The (002) crystalline plane of the carbon phase has its maximum peak at the two-theta value of 26.603°, overlapping with the most intense peak of the SnO₂ phase, corresponding to the (110) planes at 2θ=26.597°.

FIG. 8c shows shoulders for the SnO₂ diffraction peaks on the higher angle side due to Kα₁/Kα₂ XRD peak splitting, caused by the copper-based X-ray tube used in the XRD equipment, which generates radiations at wavelengths 0.1541 nm (Kα₁ line) and 0.1544 nm (Kα₂ line). The well-separated doublets in FIG. 8c can be attributed to highly crystalline SnO₂ phase formed at higher temperatures due to the grown of SnO₂ crystallite into faceted crystals.

The XRD data show the conversion of PET in molten LiCl—KCl and SnCl₂ into at least partially crystalline terephthalic acid and SnO₂ at a temperature of 500° C. and carbon and SnO₂ at temperatures of 600, 700 and 800° C.

### Example 4—Raman Characterization

Raman spectra were recorded on a Jobin-Yvon LabRam HR800 spectrometer equipped with a 488 nm laser source.

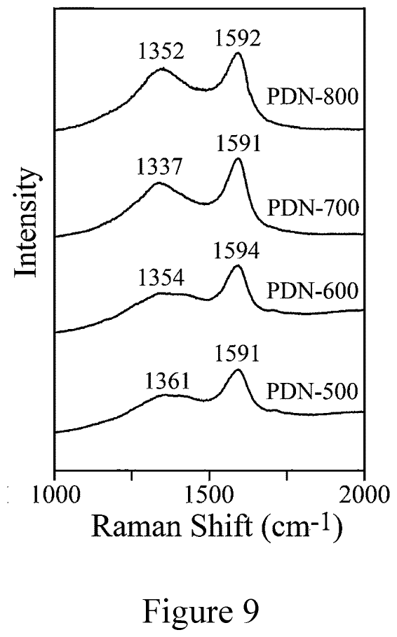

FIG. 9 shows Raman spectra of PDN-500, -600, -700 and -800 (compounds synthesized from PET, SnCl₂, and LiCl—KCl, at target temperatures of 500, 600, 700 and 800° C. as described in Example 1).

The Raman D band at 1337-1361 cm⁻¹ and G band at 1591-1594 cm⁻¹ observed in FIG. 9 confirm the presence of defective graphitic domains. The G bands observed in the spectra are the Raman signature for sp² carbons, and correspond to the presence of graphitic domains. The ratio of intensities of the D-band to the G-band (ID/IG) is an indicator of the degree of defects in graphitic domains. As observed in FIG. 9, the value of ID/IG increases with increasing temperature, indicating the development of disorder.

### Example 5—FTIR Characterization

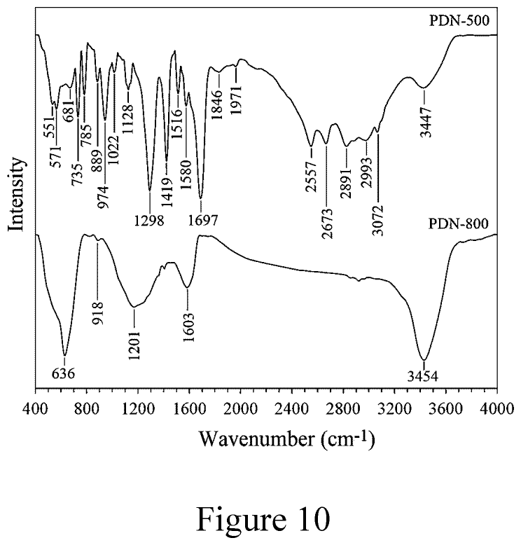

Fourier transform infrared (FTIR) spectroscopy was performed using a VERTEX 70 spectrometer within the wave range 400-4000 cm⁻¹. FIG. 10 shows FTIR spectra of PDN-500 and -800 (compounds synthesized from PET, SnCl₂, and LiCl—KCl, at target temperatures of 500 and 800° C. as described in Example 1).

The spectrum for PDN-800 had SnO₂ framework vibrations and Sn—O stretching at 636 and 918 cm⁻¹, respectively. O—H stretching signals were present at 1201, 1603 and 3454 cm⁻¹ from adsorbed water molecules.

The spectrum for PDN-500 had Sn—O stretching at 551, 571 and 681 cm⁻¹ and Sn—O—Sn vibration at 785 cm⁻¹. The spectra also contained a broad band at 3468 cm⁻¹ corresponding to O—H stretching. The spectrum for PDN-500 further contained FTIR characteristic peaks of terephthalic acid. The peak at 1697 cm⁻¹ was attributed to the asymmetric stretching vibrations of the carbonyl group (C═O), the peaks at 1419 cm⁻¹, 1298 cm⁻¹, 947 cm⁻¹, and 735 cm⁻¹ were assigned to C═C stretching, C—C stretching, O—H bending, and out of plane aromatic ring bending, respectively, which are commonly observed in the FTIR spectrum of terephthalic acid. The other FTIR peaks at 1022, 1846, 1971, 2557, 2673, 2891, 2907, 2993 and 3072 cm⁻¹ also corresponded to terephthalic acid. The FTIR peaks observed at 889 and 1128 cm⁻¹ were assigned to hydrogen bonding between oxygen of SnO₂ and hydrogen of the terephthalic acid (Sn—O—H). The peaks observed in the FTIR spectra are summarized in Table 1.

TABLE 1
Summary of the peaks observed in the FTIR spectra of samples prepared at 500 and 800° C.

| | | | Hydrogen | |
|---|---|---|---|---|
| | SnO₂ | Terephthalic Acid | bonding | H₂O |
| PDN-500 | Sn—O | Out of plane aromatic ring bending | Sn—O—H (889 | O—H stretching |
| (Sample | stretching | (735 cm⁻¹), O—H bending (947 cm⁻¹), | and 1128 cm⁻¹) | (3447 cm⁻¹) |
| prepared | (551, 571 | C—C stretching (1298 cm⁻¹), C═C | | |
| at | and 681 | stretching (1419 cm⁻¹) and carbonyl | | |
| 500° C.) | cm⁻¹) and | group (1697 cm⁻¹) + Peaks at 1022, | | |
| | Sn—O—Sn | 1846, 1971, 2557, 2673, 2891, 2907, | | |
| | vibration | 2993 and 3072 cm⁻¹ | | |
| | (785 cm⁻¹) | | | |
| PDN-800 | SnO₂ | — | — | O—H stretching |
| (Sample | framework | | | (1201, 1603 |
| prepared | vibration | | | and 3454 cm⁻¹) |
| at | (636 cm⁻¹) | | | |
| 800° C.) | and Sn—O | | | |
| | stretching | | | |
| | (918 cm⁻¹) | | | |

The FTIR results suggest the presence of terephthalic acid and SnO₂ in the sample prepared at a target temperature of 500° C.

### Example 6—Thermal Analysis

An SDT Q600 thermal analyzer equipped with alumina crucibles was used for differential scanning calorimetry (DSC), and thermal gravimetry analysis (TGA).

Generally, thermograms of terephthalic acid have partial sublimation in the form of an endothermic event at a temperature in the range of 300-400° C., followed by the decomposition of the remaining material at a higher temperature, without melting. The decomposition process is an exothermic event, leading to the formation of a gas phase (benzene, biphenyl, toluene, hydrogen and carbon monoxide) and residual carbon.

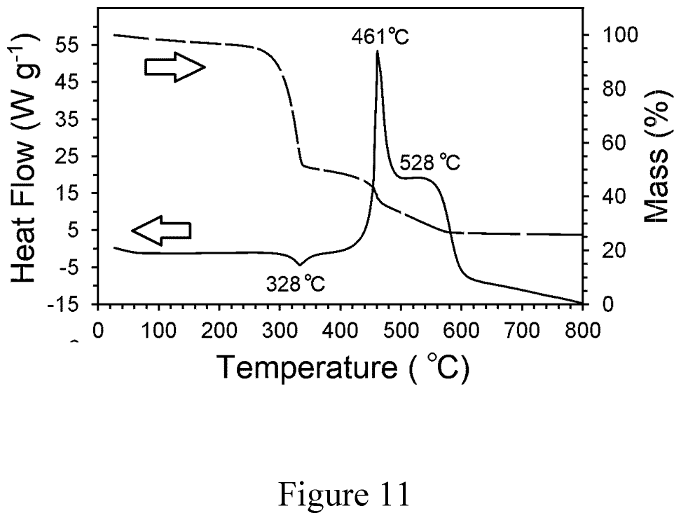

About 8 mg of PDN-500 (compound synthesized from PET, SnCl₂, and LiCl—KCl, at a target temperature of 500° C. as described in Example 1) was analyzed by DSC and TGA techniques under an air flow rate of 100 mL min⁻¹, and the results are presented in FIG. 11.

The TGA curve had a mass loss of 4.4% during the heating from room temperature to 250° C., corresponding to the removal of surface hydroxyls and/or adsorbed water. The TGA curve contained a sharp mass loss of 42.1% in the temperature range of 250-335° C. accompanied by an endothermic peak in the DSC curve at 328° C. This endothermic peak corresponded to the partial sublimation of terephthalic acid. Further heating caused a mass loss of 15.1% in the temperature range 334-465° C. in the TGA curve accompanied by an exothermic peak in the DSC thermograph at 461° C. This peak was attributed to the decomposition of the remaining terephthalic acid into gas species and residual carbon. The residual carbon was oxidized at higher temperatures, as evidenced by the exothermic peak with a maxima at 528° C., corresponding to a mass loss of 11.3% in the temperature range 462-573° C. in the TGA curve. The remaining mass of 27.1% was stable upon heating to 900° C., corresponding to the SnO₂ in the sample. With an initial moisture presence of 4.4%, the SnO₂ content of the sample was estimated as 28.3%.

The thermal analysis results suggest the presence of terephthalic acid and SnO₂ in the sample.

### Example 7—Microstructural Characterization

Morphological characterizations were conducted using scanning electron microscopy (SEM, Ultra-Plus ZEISS) as well as transmission electron microscopy (TEM, Tecnai F20).

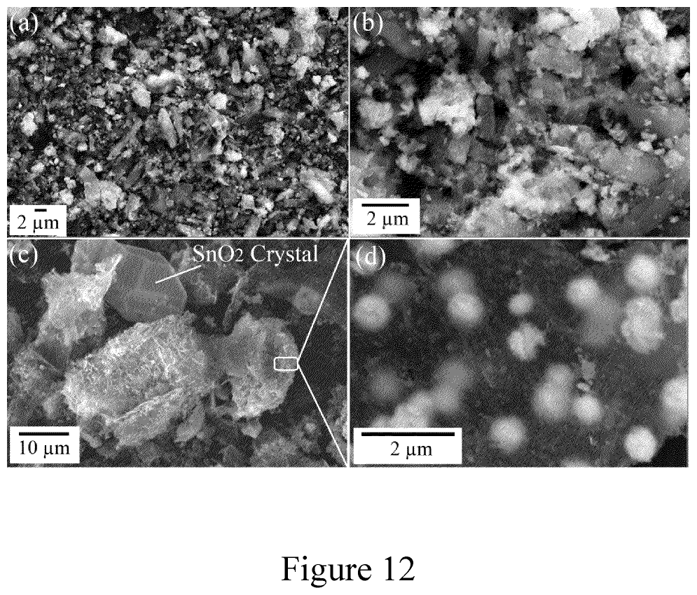

FIGS. 12a and 12b shows SEM micrographs of PDN-500 (compound synthesized from PET, SnCl₂, and LiCl—KCl, at a target temperature of 500° C. as described in Example 1). The micrographs showed that the compound contained nanostructured clusters with overall sizes of less than 2 μm. The clusters mainly contain terephthalic acid and SnO₂ based on the XRD patterns of Example 2.

FIGS. 12c and 12d shows SEM micrographs of PDN-800 (compound synthesized from PET, SnCl₂, and LiCl—KCl, at a target temperature of 800° C. as described in Example 1). The PDN-800 sample had a morphology different from that of the PDN-500 sample with large faceted crystals of SnO₂ with sizes of 10 μm. A large number of SnO₂ particles maintained a sub-micrometer sized morphology.

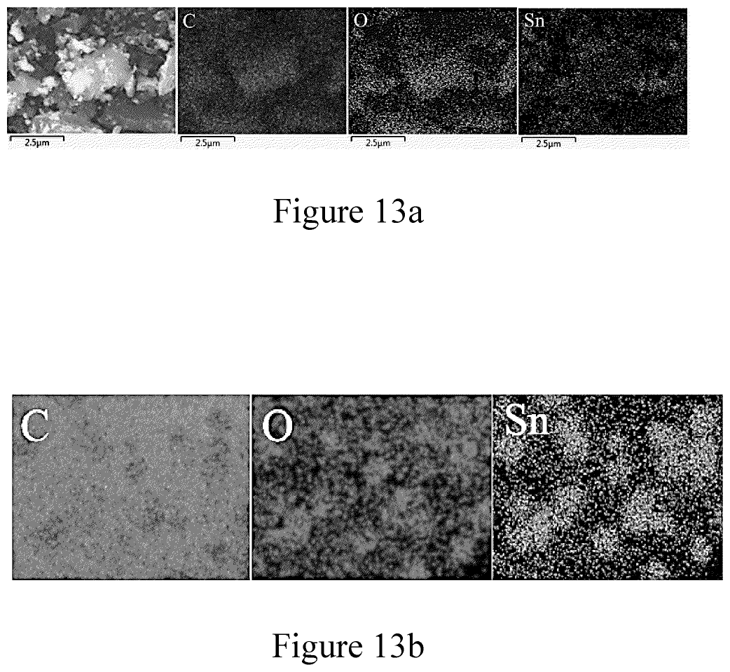

FIG. 13a shows EDS mapping of PDN-500. The relatively homogenous distribution of C, O and Sn suggests the formation of SnO₂-terephthalic acid hybrid structures. Hydrogen cannot be detected by EDS.

FIG. 13b shows EDS mapping of PDN-800. The SEM micrographs of FIG. 12d and EDS mapping of FIG. 13b indicated the presence of SnO₂ with a size of about 600 nm located in the carbon substrate formed by the carbonization of terephthalic acid.

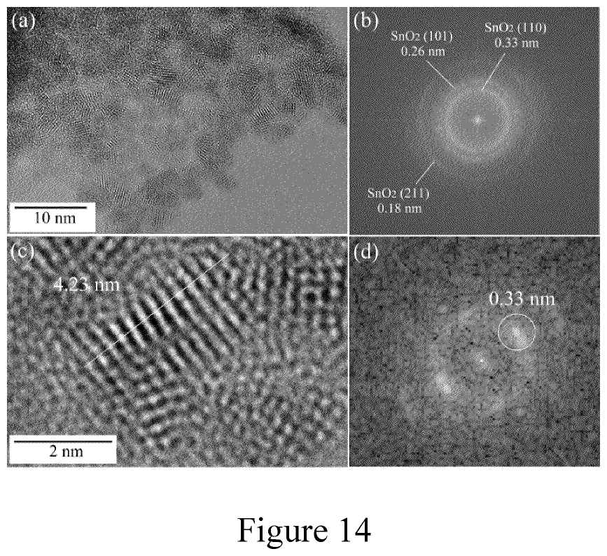

FIGS. 14a-d show TEM analysis of PDN-500. SnO₂ nanoparticles with sizes of less than 5 nm were identified in FIG. 14a. FIG. 14b shows the fast Fourier transform (FFT) pattern recorded on FIG. 14a. Rings corresponding to crystalline phases of tetragonal SnO₂ were observed in FIG. 14b. FIG. 14c shows a high magnification TEM micrograph of a nanocrystal with a length of 4.23 nm. The FFT recorded on this nanocrystal is shown in FIG. 14d and showed the presence of spots corresponding to the crystalline planes with the interlayer spacing of 0.33 nm, characteristic of the (110) SnO₂. TEM characterization of terephthalic acid was not possible due to its instability under the high voltage electron beam applied.

### Example 8—Surface Area Characterization

Brunauer-Emmett-Teller (BET) method was employed to evaluate the surface area of samples.

The BET specific surface area of PDN-500 was determined to be 19.2 m² g⁻¹.

### Example 9—XPS Characterization

X-ray photoelectron spectroscopy (XPS) of the compounds synthesized from PET and molten LiCl—KCl and PET, SnCl₂, and LiCl—KCl, at a target temperature of 500° C. (PDN-500) as described in Example 1 was measured using an XPS equipment (ESCALAB250, Thermo Fisher Scientific).

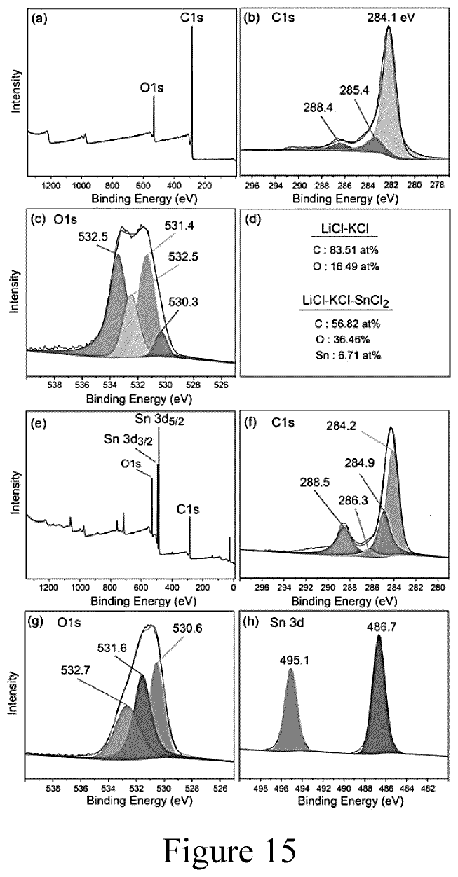

XPS spectra of the PET+LiCl—KCl sample is shown in FIGS. 15a-c. The spectra in FIG. 15a indicate the existence of C and O in the sample. As shown in FIG. 15b, the C is core-level spectrum is dominated by the peak at 284.1 eV, representing the presence of graphitic carbon with C—C bonding. The presence of the two broadening peaks centered at 285.4 and 288.4 eV is representative of the lattice disruption caused by random orientations of dangling bonds with respect to the carbon atoms, and defective carbon, respectively. The XPS observations match the XRD results of Example 2, further confirming that the PET+LiCl—KCl sample contains amorphous carbon. The curve fitting associated with the 0 is peak of the XPS spectrum of the sample (FIG. 15c) showed the existence of three types of surface oxygen bonding, which were assigned as C═O (530.3 eV), C—O, carbonyl (532.5 eV), C—OH and O—C═O (533.4 eV). The amount of surface oxygen in the sample was calculated as 16.49 atomic percentage (at %), which is comparable with that of amorphous carbons.

XPS spectra of the PDN-500 sample is shown in FIG. 15e-h. Compared to FIG. 15a, the spectra of FIG. 15e included the Sn peaks and a much lower ratio of carbon to oxygen due to the formation of terephthalic acid and SnO₂. The C is spectrum of PDN-500, shown in FIG. 15f, had a peak that could be split into four peaks at 284.2, 284.9, 286.3 and 288.5 eV, corresponding the four different types of nonequivalent carbon atoms observed in terephthalic acid corresponding to the excitations of the phenyl carbon into the π* molecular orbital. The core-level O 1s spectra, shown in FIG. 15g, could be separated into three peaks located at 530.6, 531.6 and 532.7 eV, corresponding to the O—Sn, C—O—Sn and chemisorbed oxygen, respectively. The peak at 532.7 eV is attributed to excitation from the carbonyl bond in terephthalic acid. The Sn 3d spectrum of the sample, shown in FIG. 15h, has peaks at 486.7 eV and 486.7 indicating the existence of Sn 3d_{5/2} and Sn 3d_{3/2}, respectively. These peaks are attributed to characteristics of Sn⁴⁺, indicating the formation of SnO₂, which is in agreement with the XRD results of Example 2. The Sn content at the surface was obtained as 6.71 at %.

FIG. 15d shows the results of the elemental analysis of each sample.

### Example 10—Bulk Electrical Conductivity

The room temperature electrical conductivity was measured by a four-probe system (DCY-3F, Hunan Zhenhua Analysis Co. Ltd.) equipped with a vertical unidirectional hydraulic press. The evaluations were conducted by compressing 2.0 g of sample into an acrylic tube (ID=20.05 mm, H=45.37 mm) using a copper piston (D=20.05 mm, H=85.36 mm) on a copper holder, at different pressure values up to about 6 MPa using a hydraulic press. At different pressures, various values of electric current in the range 0.10-0.30 A were conducted between the copper piston and holder, and the corresponding potentials were recorded using the four-probe DC method at 20° C. The control system had a display with voltage and current resolution of 0.1 mV and 0.1 mA, respectively. The values of powder density could then be calculated by applying different values of pressure on the samples. The electrical resistance could be calculated from the slope of the V-I curves. The resistivity (ρ) of the samples was then calculated as follows:

$$ρ = \frac{R \times S}{h} \qquad ( 5 )$$

where R is the electrical resistance obtained from the slope of voltage vs. current, S is the cross-sectional area of the sample pellet (D=2 cm) and h is the height of the sample pellet. By inversing the resistivity of sample, the electrical conductivity (σ) could be calculated as follows:

$$σ = \frac{1}{ρ} . \qquad ( 6 )$$

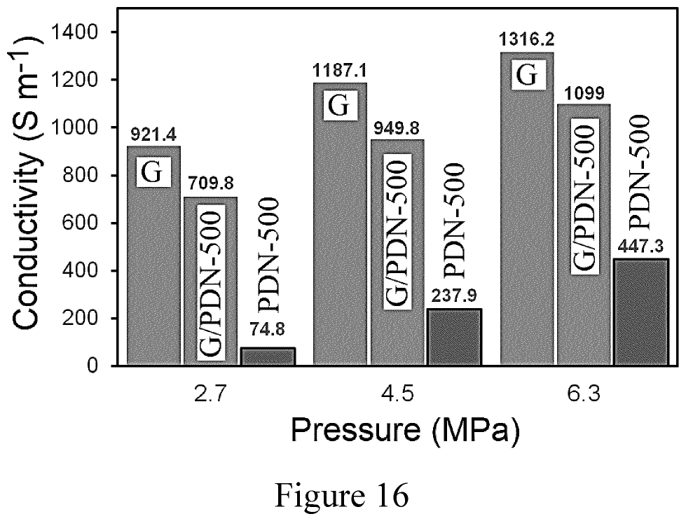

To measure the electrical conductivity values, PDN-500 was thoroughly mixed with synthetic graphite powder (G) with the mass ratio of 25:75, and the mixtures obtained were used for the conductivity measurements. For the measurement, 2.0 g of the sample (G or G+PDN-500 mixture) was fed into the cavity of the electrical measurement device and the values of electrical conductivity for G (C_{G}) and G+PDN-500 mixture (C_{G+PDN-500}) were measured at various compressive pressures. The presence of graphite powder allowed to measure the bulk electrical conductivity of PDN-500 (C_{PDN-500}) using the equation (7), and the results are shown in FIG. 16.

$${C}_{G + P D N - 500} = {C}_{G} \times G mass \% + {C}_{P D N - 500} \times PDN - 500 mass \% \qquad ( 7 )$$

As observed from FIG. 16, the bulk electrical conductivity of PDN-500 increased with the increase in applied pressure from 74.8 S m⁻¹ under a pressure of 2.7 MPa to 447.3 S m⁻¹ at 6.3 MPa. Additionally, the bulk density of PDN-500 was calculated by measuring the height of the compressed powder (and then its volume), with this consideration that values of the cross sectional area of the compressed powder and its mass remain constant during the uniaxial compression test. The bulk density of PDN-500 was obtained to be 1.2 g cm⁻³ at 6.3 MPa.

### Example 11—Li-Ion Storage Performance and Electrochemical Characterization

Slurry suspensions were made using the prepared samples (80 wt. %) mixed with conductive carbon (C65, 10 wt. %) and PVDF binder (10 wt. %) by the application of NMP solvent. The slurry suspensions were coated on copper foils, then vacuum dried at 100° C. overnight. Then, the copper foils loaded with active materials were cut into circular pieces with the diameter of 1.2 cm, and assembled into CR2025 half-cells. In this electrochemical cell, the coated copper foil functioned as the working electrode, while a Li disc functioned as both the reference electrode and the counter electrode. Meanwhile a glass microfiber separator (Whatman, 1823025) was placed between the above mentioned electrodes to prevent physical contact between them, while facilitating ion transport in the cell. The cell was assembled in an atmosphere controlled glove-box (Mikrouna) filled with high purity argon with 02 and H₂O contents of less than 0.1 ppm. The active material mass loading was measured as 1.3 mg cm⁻². The same mass loading was used to evaluate galvanostatic cycling performances, and also for the cyclic voltammetry measurements. The electrolyte was LiPF₆ (1 M) in ethylene carbonate (EC), dimethyl carbonate (DMC), and ethylmethyl carbonate (EMC) with 1:1:1 volume ratio. The coin cells were pressed using a punch machine (YLJ-24 T, MTI corporation). The half-cells were allowed to stabilize at room temperature for 10 h, and then assembled on a battery test system (Land CT2001A) to perform the charge and discharge processes in the voltage range 0.01-3.0 V (vs Li⁺/Li) at constant and variable current densities. The cyclic voltammetry (CV) measurement and electrochemical impedance spectroscopy (EIS) were conducted using a CHI-660E electrochemical workstation

The Li-ion storage performance of PDN-500, PDN-600, PDN-700 and PDN-800 were evaluated through constant current galvanostatic charge/discharge experiment and cyclic voltammetry using the half-cell configuration employing Li as both the counter and reference electrodes at the voltage range 0.01-3 V (vs Li⁺/Li) for 500 cycles.

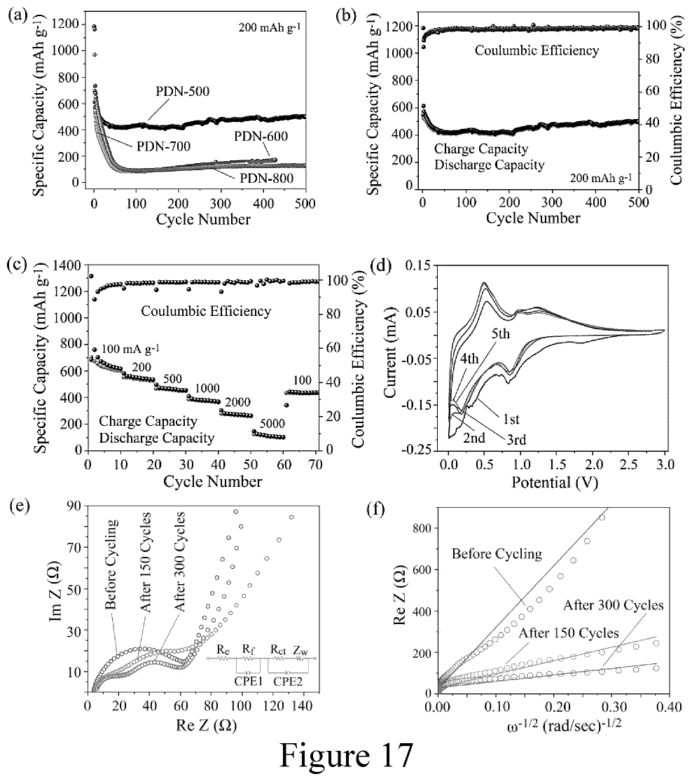

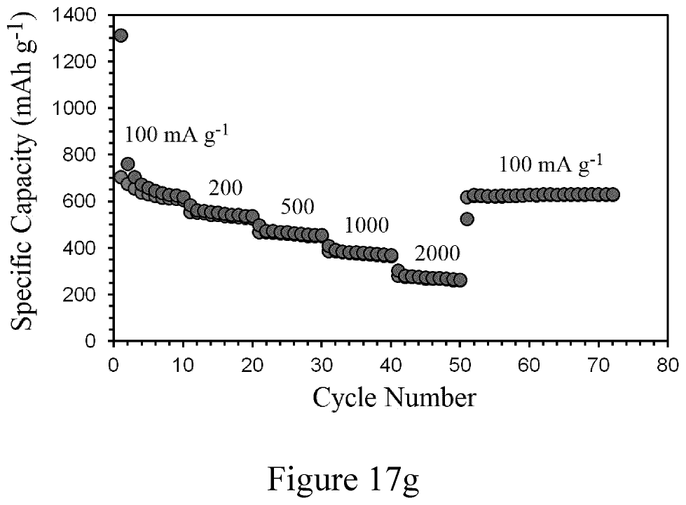

The discharge capacity values over the Li-ion insertion/extraction cycles into/out of PDN-500, -600, -700 and -800 at the current density of 200 mA g⁻¹ are shown in FIG. 17a. PDN-500 exhibited a discharge capacity of 498 mAh g⁻¹ after 500 cycles and was substantially greater than the discharge capacities of PDN-600 (169 mAh g⁻¹, 428^{th} cycle), PDN-700 (138 mAh g⁻¹, 360^{th} cycle) and PDN-800 (125 mAh g⁻¹, 500^{th} cycles).

The coulombic efficiency of the electrode made of PDN-500 at a current density of 200 mA g⁻¹ is shown in FIG. 17b. The first discharge/charge cycle had capacity values of 1183/545 mAh g⁻¹, providing a coulombic efficiency of 46.1%. The capacity loss was assigned to irreversible decomposition of the electrolyte on the surface of the electrode, leading to the formation of solid electrolyte interphase (SEI). The second discharge/charge cycle had capacity values of 612/535 mAh g⁻¹, giving a higher coulombic efficiency of 87.5%, showing the limited interaction of the electrolyte with the electrode during the second cycle. The third discharge/charge had capacity values of 572/524 mAh g⁻¹, giving a coulombic efficiency of 91.5%. The coulombic efficiency gradually increased with the cycling, providing a value of 97.3% after 22 cycles and 99.2% after 78 cycles. The coulombic efficiency fluctuated slightly until discharge/charge capacity values of 498/492 mAh g⁻¹ were recorded after 500 cycles, corresponding to a coulombic efficiency of 98.8%.

The Li-ion storage capacities of the waste plastic derived PDN-500 were measured at current densities between 100-2000 mA g⁻¹, as shown in FIG. 17g. The electrode delivered reversible capacities of 605.7 (10^{th} cycle), 527.2 (20^{th} cycle), 449.4 (30^{th} cycle), 363.3 (40^{th} cycle) and 260.5 mAh g⁻¹ (50^{th} cycle) at current densities of 100, 200, 500, 1000 and 2000 mA g⁻¹, respectively. Then, by returning the current density back to 100 mA g⁻¹, a reversible capacity of 628 mAh g⁻¹ was recorded at the 70^{th} cycle. This result confirms that the rate performance of PDN-500 electrode was desirable at current densities up to 2000 mA g⁻¹.

The Li-ion storage capacities of the PDN-500 electrode were measured at current densities between 100-5000 mA g⁻¹, as shown in FIG. 17c. After cycling at current densities of 100, 200, 500, 1000 and 2000 mA g⁻¹, the electrode delivered a capacity of 101.3 mAh g⁻¹ under 5000 mA g⁻¹ at the 60^{th} cycle. Upon returning the current density back to 100 mA g⁻¹, a reversible capacity of 436.8 mAh g⁻¹ was recorded at the 70^{th} cycle, which was smaller than the value of 600.7 mA g⁻¹ recorded at the 10^{th} cycle at the same current density. A relatively high capacity of 436.8 mAh g⁻¹ could still be delivered after 70 cycles and after a high current density of 5000 mA g⁻¹, suggesting relatively high rate performance of the material at current densities up to 5000 mA g⁻¹.

Cyclic voltammetry (CV) curves of PDN-500 recorded at different cycles are shown in FIG. 17d. The peaks observed in the CV curves represent electrochemical reactions involved during the battery cycling. In the first cathodic scan, a peak was detected at around 1.7 V vs. Li/Li⁺, which was not seen in the subsequent cycles. This peak was attributed to the formation of the SEI layer due to the reduction of the solvent (EC-DEC-DMC) on the electrode, reaction (8).

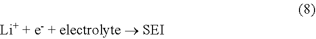

The prolonged cycling capability of electrodes requires the presence of a stable SEI layer.

Furthermore, the cathodic peaks observed at around 0.83 and 0.22 V were attributed to the transformation of SnO₂ to Sn, reaction (2a), and the alloying reaction between lithium and tin, leading to the formation of Li—Sn intermetallics, as shown in reaction (2b). The oxidation peak at around 0.54 V present during the first anodic cycle, was attributed to the lithium de-alloying of LiₓSn, the reaction (2b). The anodic peaks at around 0.97 and 1.24 V corresponded to the reversible transition from Sn to SnO₂ as shown in reaction (2a).

As shown in FIG. 17d, the second cathodic cycle was different from the first one in that there was no obvious cathodic peak at around 1.7 V in the second cycle, demonstrating the relatively high stability of the SEI layer formed during the first cycle. Moreover, the peaks associated with the reduction and oxidation of tin compounds were observed in the CV voltammograms recorded at subsequent cycles, demonstrating the reversibility of lithiation/dilithiation of SnO₂ nanocrystals embedded in terephthalic acid. Under this condition, terephthalic acid acted as the support for the accommodation of volume changes involved in the electrochemical reactions (2a) and (2b), which ensured the prolonged cyclability of the electrode.

Electrochemical impedance spectroscopy (EIS) measurements were performed on the PDN-500 electrode before cycling, and after 150 and 300 galvanostatic discharge/charge cycles at the current density of 200 mA g⁻¹. The electrochemical impedance spectra recorded at the frequency range from 10 mHz to 100 kHz at the amplitude of 5 mV, as well as the equivalent circuit for the Nyquist plots are shown in FIG. 17e. The spectra exhibited semicircles in the high frequency region and a sloping straight line in the low frequency range. The electrochemical behavior of PDN-500 at the high-range frequency region represented the resistance of the lithium ion transfer through the SEI film (R_{f}), and that at the medium-range frequency region was attributed to the charge transfer resistance of the active material (R_{ct}). On the other hand, the low-frequency impedance of the electrode was be attributed to the Warburg impedance (Z_{w}), representing the immigration of lithium ions. The equivalent circuit shown in FIG. 17e also contains constant phase element (CPE) components that model the behavior of a non-ideal capacitor. As can be observed from the spectra, the semicircle obtained after 150 cycles had a lower diameter than that of the fresh electrode, confirming the reduction of R_{ct} over cycling. The reduction of the cell resistance was attributed to the rearrangement of SnO₂ nanoparticles in the terephthalic acid matrix over cycling.

The electrochemical parameters derived from the EIS spectra in FIGS. 17e and 17f are shown in Table 2. From the measured values, the lithium ion diffusion coefficients were calculated using equation (9), and the results are shown in Table 2.

$${D}_{L i} = \frac{{R}^{2} \times {T}^{2}}{2 \times {A}^{2} \times {n}^{4} \times {F}^{4} \times {C}^{2} \times {σ}^{2}} \qquad ( 9 )$$

where R is the gas constant (8.314 J mol⁻¹ K⁻¹); T is the temperature (298 K); A is the surface area of the electrode (˜1.13 cm²); n is the molar number of electrons transferred (1 for lithium); F is the Faraday constant (96,485 C mol⁻¹); C is the concentration of Li-ions derived from tapping density of the active material, and a is the Warburg factor determined from the slope of real Z vs ω^{-1/2} shown in FIG. 17e.

TABLE 2
Electrode kinetic parameters obtained from the
equivalent circuit fitting of Nyquist plots

| Cycle | Re (Ω) | Rct (Ω) | Rf (Ω) | σ (Ω s^{-1/2}) | D_{Li} (cm²s⁻¹) |
|---|---|---|---|---|---|
| 0 | 3.47 | 34.06 | 6.41 | 600.30 | 2.80 × 10⁻¹¹ |
| 150 | 3.58 | 22.83 | 8.81 | 283.44 | 1.26 × 10⁻¹⁰ |
| 300 | 5.40 | 25.54 | 15.21 | 108.83 | 8.51 × 10⁻¹⁰ |

From Table 2, the electrolyte resistance (Rₑ) slightly increased from 3.47Ω in the non-cycled electrode to 3.58Ω after 150 cycles. This value considerably increased to 5.40Ω after 300 cycles. On the other hand, the charge transfer resistance (R_{ct}) considerably decreased from 34.06Ω to 22.83Ω after 150 cycles. This value slightly increased to 25.54Ω after 300 cycles. Also, the resistance of SEI film (R_{f}) slightly increased from 6.41Ω to 8.81Ω after 150 cycles, which further increased to 15.21Ω after 300 cycles. The Warburg coefficient, σ, was determined from the slope of the real impedance versus the reciprocal of the square root of the angular frequency (FIG. 17f). This value decreased from 600.30 s^{-1/2} in the non-cycled electrode to values of 283.44 and 108.83 s^{-1/2} in the 150- and 300-cycled electrode, respectively. A lower value of Warburg coefficient indicates a higher value of ion diffusion rate (D_{Li}) as can be observed in Table 2. The lithium diffusion rate reduces from the initial value of 2.80×10⁻¹¹ to 1.26×10⁻¹⁰ cm² S⁻¹ after 150 cycles.

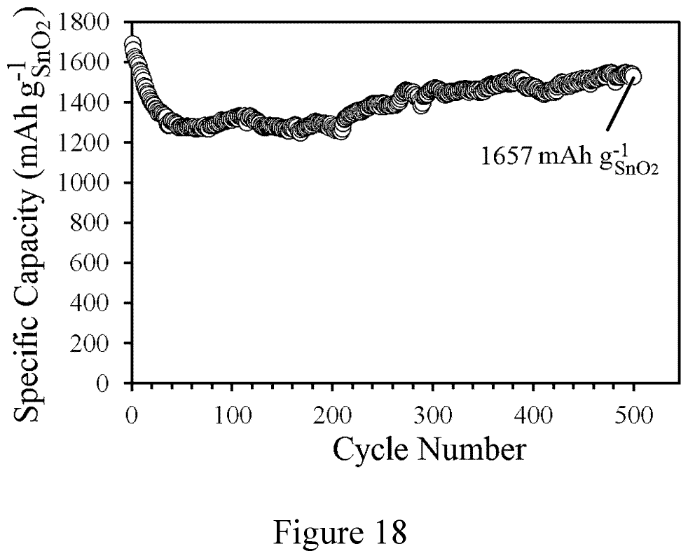

FIG. 18 shows the prolonged cycling performance of the electrode made of the sample (PET+(SnCl₂−LiCl−KCl)/500° C.) based on the mass of the SnO₂ (oxide phase) present the electrode measured at current density of 200 mA g⁻¹. The thermal analysis of PDN-500 from Example 6 indicates that PDN-500 contained around 28.3 wt. % SnO₂. The capacity of the SnO₂ component of the electrode could be obtained based on the capacity of terephthalic acid (14 mAh g⁻¹), and the conductive carbon employed for the preparation of the electrode (184 mAh g⁻¹). Accordingly, the capacity of the electrode was translated to be 1657 mAh per gram of SnO₂ which is approximately the theoretical capacity of SnO₂, demonstrating that that the SnO₂ nanocrystals exhibited their maximum capacity even after 500 cycles when incorporated into the terephthalic acid matrix.

### Example 12—Morphological Characterization of Electrodes After Cycling

Morphological characterization of the electrodes used in Example 11 after cycling were performed with the methods of Example 7.

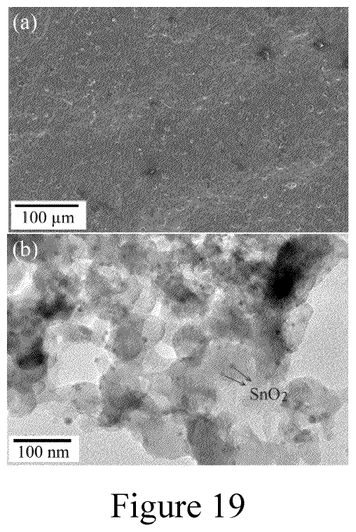

FIG. 19a shows a SEM micrograph of a PDN-500 electrode after 200 Li insertion/extraction cycles. The lack of obvious cracks was evident, showing the structural integrity of the material over cycling.

FIG. 19b shows a TEM micrograph of the PDN-500 electrode. Fine SnO₂ particles were dispersed within the terephthalic acid matrix. While the presence of holes is evident, the material maintained its structural integrity. The SnO₂ particles were entirely covered by the matrix, which enhanced the integrity of the electrode.

FIGS. 19a and 19b confirmed that that presence of terephthalic acid in PDN-500 was effective in preventing the electrode material from being disintegrated during the prolonged battery cycling.

### Example 13—Pseudocapacitive Performance

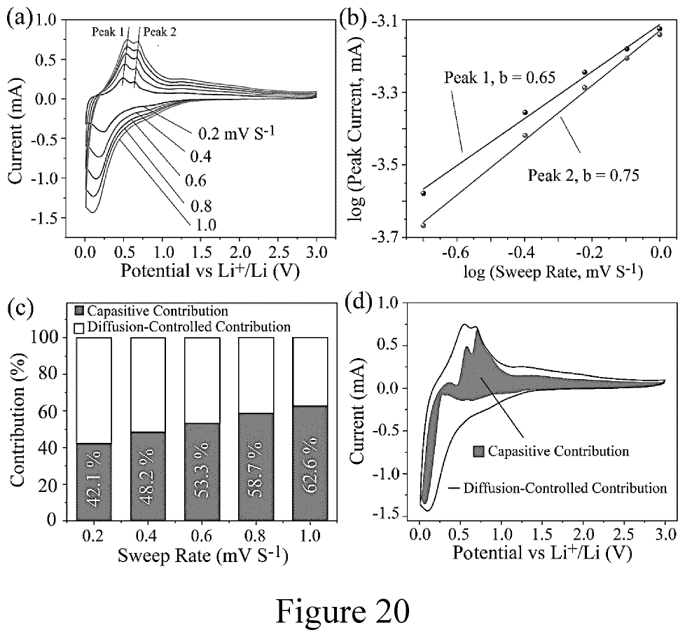

To evaluate the contribution of pseudocapacitive Li-storage to the cycling performance of PDN-500, sweep voltammetry measurements were carried out on the cell after 100 cycles at different scan rates of 0.2, 0.4, 0.6, 0.8 and 1.0 mV s⁻¹, as described in Example 11, and the results are shown in FIG. 20a. Two anodic peaks were detectable in the CV curves, corresponding to the dilithiation process of the electrode. These peaks shifted to higher potential values with increasing the sweep rate from 0.2 to 1.0 mV s⁻¹, which indicated the limitation of the ion diffusion. Moreover, the intensity of the peaks increased with increasing sweep rate. The current intensity (i) and the sweep rate (v) can be related by the power law shown as Eq. (10) and its logarithmic form of Eq. (11):

$$i = a \times {v}^{b} \qquad ( 10 )$$

$$log I = log a + b \times log v \qquad ( 11 )$$

where a and b are dimensionless variables. For a redox reaction limited by a semi-infinite diffusion-controlled process, b can have a value of 0.5, while that of the capacitive process would be close to unity. By plotting the log peak current vs log scan rate (FIG. 20b), the value of b for Peaks 1 and 2 in FIG. 20a were calculated as 0.65 and 0.75, respectively. The value of 0.65 is closer to 0.5, and therefore, the reaction taking place at Peak 1 is likely to be governed mostly by a diffusion-based process in addition to a limited contribution from capacitive charge storage processes. The second peak, with the b value of 0.75, corresponds to the capacitive behavior with limited contribution from diffusion-based processes. Based on the results obtained, the relative contributions of capacitive and diffusion-controlled processes at different sweep rates are exhibited in FIG. 20c, illustrating that the pseudocapacitive contribution gradually increases with the increase of the scan rate. FIG. 20d shows the capacitive contribution to the total current at 1.0 mV s⁻¹.

### Example 14—Oxygen-Free Synthesis

PET plastic pieces (10 g, polymeric material) were mixed with SnCl₂ (5.05 g, depolymerization agent) as well as KCl (13.79 g) and LiCl (11.53 g). The mixture was heated to a maximum temperature of 500° C. with a heating rate of 10° C. min⁻¹ and a duration of 10 min at maximum temperature, then the temperature was reduced to room temperature. The heat-treatment process was conducted in a tube furnace under gas stream of Ar (95%) and H₂ (5%). Then the materials obtained were washed with deionized water, and the suspension obtained was vacuum filtered using a polymer filter paper to collect the filtrate, which was dried afterward at 100° C. for a few hours.

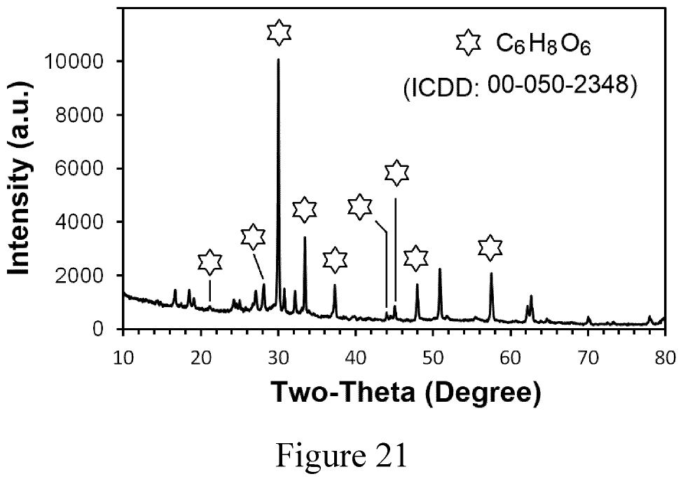

The XRD pattern of the product obtained is shown in FIG. 21, exhibiting the presence of terephthalic acid (C₆H₈O₆). Other phases arising from the depolymerization agent (SnCl₂) may also have be formed during the heat-treatment including SnO₂, SnO and Sn.

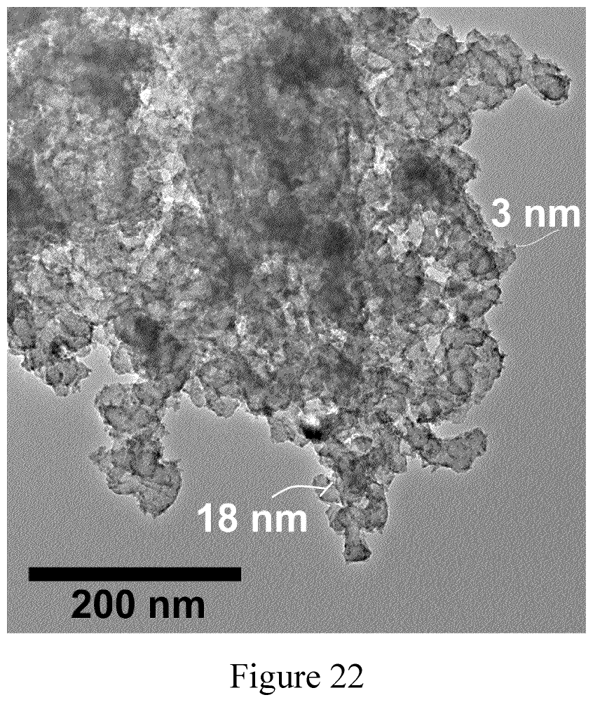

The TEM micrograph recorded on the organic compound is shown in FIG. 22, demonstrating the nanostructured feature of the material. Particles with sizes of 18 and 3 nm were identified in the micrograph.

### Example 15—Silicon-Containing Materials

Silicon particles with sizes of less than 500 μm (21.7 g) were ball milled for 50 hours using a planetary ball billing machine together with zirconia balls (D=15 mm, 650 g) in the presence of n-hexane at a rotational speed of 300 rpm.

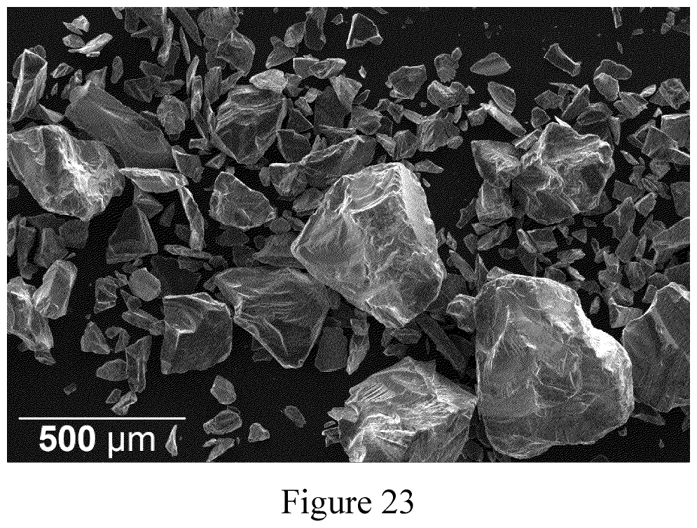

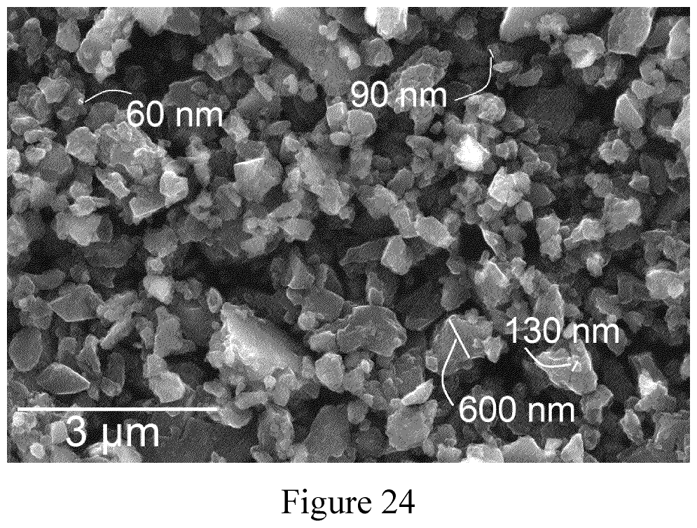

FIG. 23 shows the SEM micrograph of the raw Si material. After ball milling, the ball-milled Si was dried to remove n-hexane. FIG. 24 shows the SEM micrograph of the resulting Si, indicating that the particle sizes of the ball-milled Si were reduced to around 50 nm-700 nm.

The resulting SiNPs (2 g) were mixed with PET particles (4 g) and SnCl₂ (4 g). The mixture was placed in an alumina crucible, and the crucible was placed inside a muffle furnace, and heated under an air atmosphere to 500° C. with a heating rate of 10° C. min⁻¹ and a holding time at maximum temperature of 5 min. Then, the furnace was cooled down to the room temperature, and the material obtained was washed with deionized water, followed by vacuum filtration and drying.

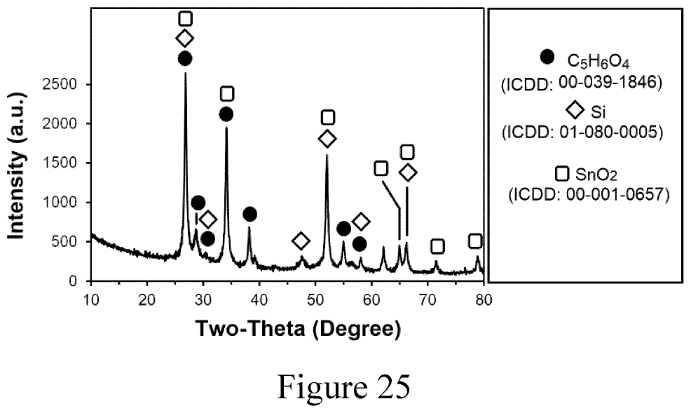

The XRD pattern of the product is shown in FIG. 25, from which the presence of terephthalic acid (C₅H₆O₄) is evident. Furthermore, FIG. 25 demonstrates the presence of Si and SnO₂.

The SEM morphology of the resulting product is shown in FIGS. 26a and 26b. From the micrograph, the terephthalic acid integrated SiNPs (with particles sizes of around 50-700 nm) into Si/SnO₂/terephthalic acid nanostructured composite material with particle sizes of larger than 10 μm.

### Example 16—Comparison Between Nanostructured and Commercial Terephthalic Acid

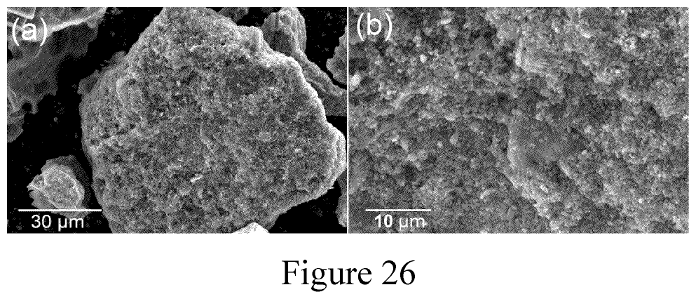

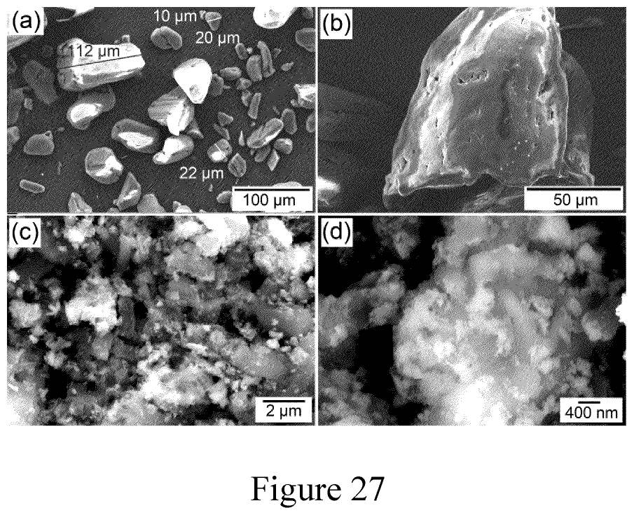

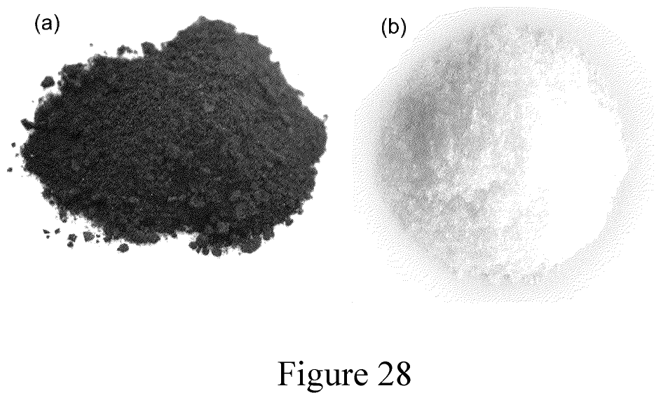

PET was nanostructured using SnCl₂ as the depolymerization agent in the presence of LiCl—KCl according to the Example 1. The nanostructured material was characterized to be the mixture of nanostructured terephthalic acid (C₈H₆O₄) and SnO₂ nanocrystals. Micrometer-sized terephthalic acid powder (Shanghai Macklin Biochemical Co., 400-623-8666, 99%) was characterized as a comparison to the composition of Example 1. FIG. 6d shows the XRD pattern of the commercial terephthalic acid, providing evidence for the crystalline structure of the material. FIGS. 27a-d exhibit the SEM morphology of the material, showing the presence of particles with sizes in the range of around 10-100 μm. FIGS. 28a and 28b show powders of PDN-500 (PET+(SnCl₂−LiCl−KCl)/500° C.) and the commercial terephthalic acid, respectively. The heat-treatment of PET in molten salt environments led to the formation of terephthalic acid, with the same crystalline structure, but substantially different morphology, microstructure and color than those of commercially available terephthalic acid. PDN-500 had a dark color, whereas the commercial terephthalic acid was white, as shown in FIGS. 28a and 28b, respectively. Considering the white color of SnO₂ nanoparticles, the dark appearance of the nanostructured material may be due to the black color from terephthalic acid. Since commercial terephthalic acid was a white crystalline solid, the black appearance of the terephthalic acid in PDN-500 may be related to the nanostructured nature of the material.

### Example 17—Heat Treatment of PET, SnCl₂, and Li-KCL Under Ar Atmosphere

60 g PET pieces were mixed with 184 g SnCl₂, 46.4 g LiCl and 54.9 g KCl. The mixture was placed inside an alumina crucible (H=10 cm, D=7.5 cm), and the crucible was covered with an alumina lid, and placed into a stainless steel retort equipped with gas inlet/out. The retort was heated in an electric furnace, under a flow of Ar gas to a maximum temperature of 445° C. with an average heating rate of 5° C./min, while the temperature of the materials inside the crucible was measured by a thermocouple placed inside the crucible. After reaching the maximum temperature, the heating was terminated, followed by natural cooling of the retort to room temperature.

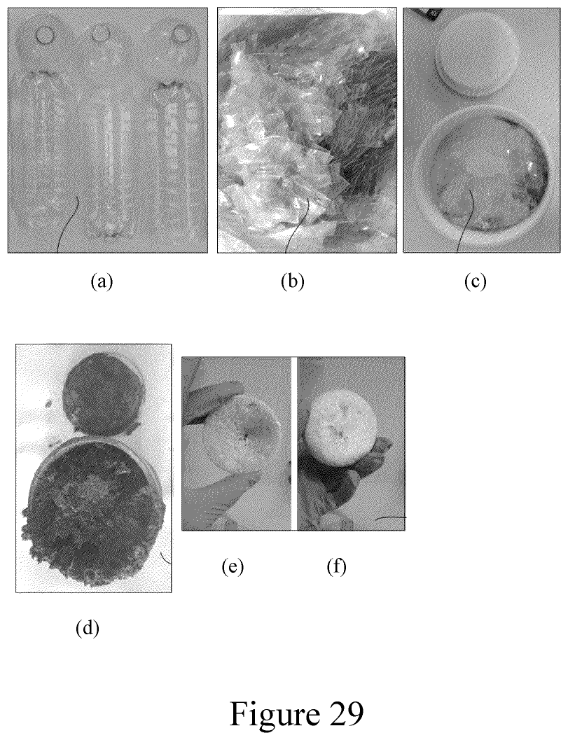

FIGS. 29a-d shows photographs of waste PET, shredded PET, as well as the mixture of PET+SnCl₂+LiCl+KCl loaded alumina crucible before and after heat-treatment at 445° C., respectively. As can be observed, the product obtained after the heat-treatment had a considerably larger volume than the initial materials, indicating that the product possessed a porous structure. The porous product could easily be separated from the crucible. Since the apparent density of salts was greater than the porous product, the salt mainly stuck to the bottom of the crucible during the molten salt process. A solidified salt disc was present at the bottom of the crucible, as shown in FIGS. 29e-f This observation suggests that SnCl₂−LiCl−KCl can be separated from the porous product based on its density, promoting the facile collection of the product without washing the main body of the salt mixture.

Based on these observations, it is believed that the depolymerization of PET occurs at the interface between the salt and PET, and the depolymerized organic compound moved toward the upper part of the reactor, so that at the end of the process, the organic compound was dominantly positioned at the upper part, and the salt at the bottom of the reaction container. This positioning can substantially ease the separation process.

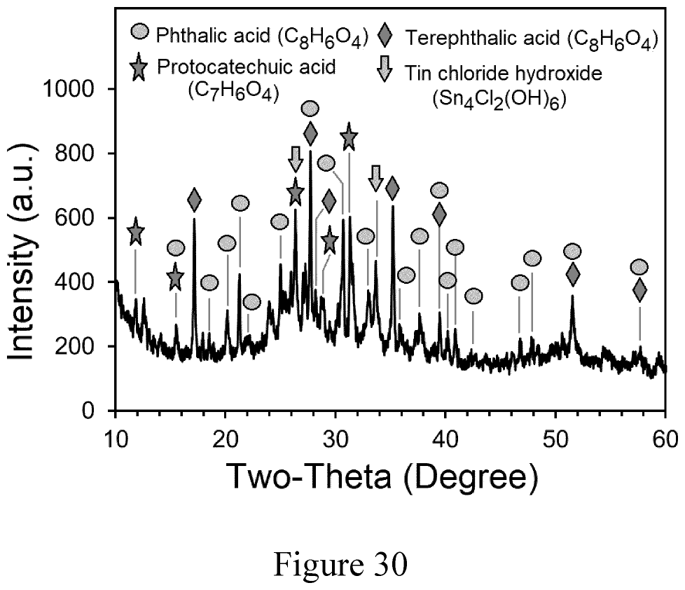

The material obtained at the upper part of the crucible was washed with water to remove remaining salt trapped within its porous structure, and the material obtained was dried at 80° C. for 2 hours. FIG. 30 shows the XRD pattern of the material obtained. As can be observed, the product contained various crystalline organic compounds including phthalic acid (C₈H₆O₄) with monoclinic crystalline structure (ICDD: 00-037-1919), terephthalic acid and protocatechuic acid (C₇H₆O₄; ICDD: 00-008-056), in addition to tin chloride hydroxide. The latter was likely formed during the washing step, through the hydrolysis of remaining SnCl₂.

### Example 18—Heat Treatment of PET, SnCl₂, and Li-KCL Under Ar+H₂ Atmosphere

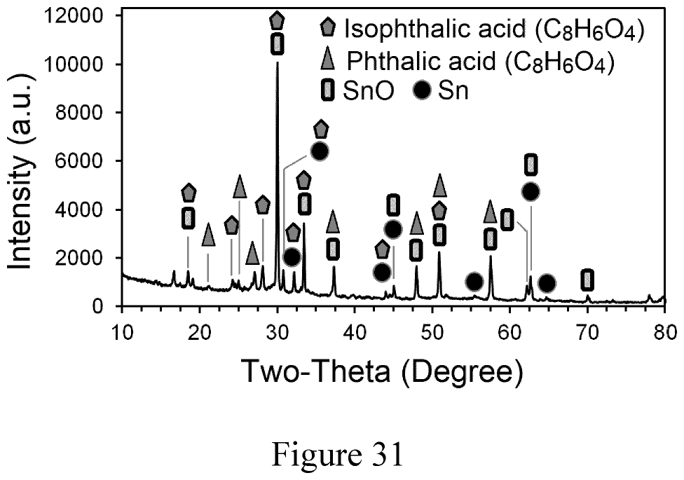

A mixture containing 13.79 g KCl, 11.53 g LiCl, 5.05 g SnCl₂ and 10 g plastic pieces was placed in an alumina crucible, and the crucible was placed in a tube furnace equipped with an alumina tube. The tube was subjected a flow of Ar-5% H₂ gas through the tube. The temperature was raised to 90° C. (8 h) and then with a heating rate of 10° C./min to 500° C. Then, the sample was maintained at 500° C. for 10 min before the temperature was reduced to room temperature. The heat-treatment was conducted under the flow of Ar-5% H₂. The product obtained was then washed with deionized water, and subsequently vacuum filtered using a polymer filter. The filtrate was then dried at 100° C. for 24 h. The XRD pattern of the product recorded using Cu-K_{α} (λ=0.1542 nm) is shown in FIG. 31. The pattern could be indexed to crystalline organic compounds such as isophthalic acid (C₈H₆O₄, ICCD: 00-037-1920) and phthalic acid (C₈H₆O₄, ICCD: 00-037-1919) both having monoclinic structure, in addition to SnO (ICCD: 01-085-0712) and metallic Sn (ICCD: 00-004-0673).

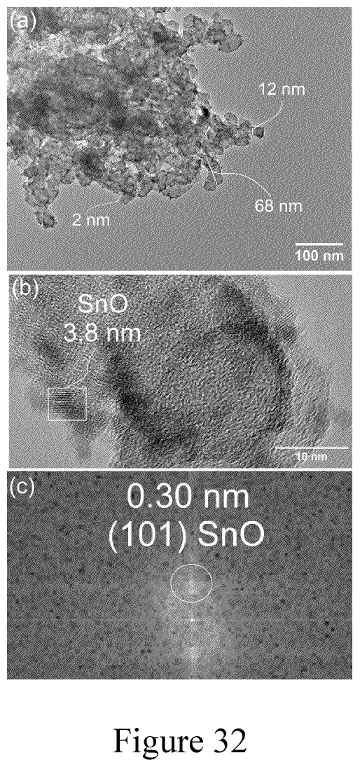

FIGS. 32a-c shows TEM micrographs of the product obtained by heating the mixture in Ar-5% H₂, followed by washing and drying steps. FIG. 32a shows a bright field TEM micrograph showing that the crystalline organic compounds contains agglomerated particles made of nanoparticles with sizes in the range of around to 1 to 100 nm. Nanoparticles with sizes of 2, 12 and 68 nm can be observed in the micrograph. FIG. 32b shows a SnO nanoparticle with the size of 3.8 nm embedded into the organic compounds. FIG. 32c shows the fast Fourier transform pattern recorded on the SnO nanoparticle, presenting the spots corresponding to (101) crystalline planes of SnO with tetragonal structure.

### Example 19—Water Purification

The product from Example 18, which contained crystalline organic compounds, SnO and Sn, was used as an adsorbent/photocatalyst for the removal of organic dyes from aqueous solutions. The photocatalytic and adsorption performances of the product were characterized in a closed metallic box at room temperature. 0.1 g product was added into 100 mL methyl orange (MO) or methylene blue (MB) solutions with a concentration of 50 mg/L. The suspension was subjected to magnetic stirring at various durations in the dark and under a LED light source module with a fixed wavelength of 450 nm in the visible region. A water cooling jacket was used to maintain the reaction temperature at 20° C. At specific intervals, volumes of 2 mL were extracted and filtered into a cuvette and characterized using a UV-Vis spectrophotometer.

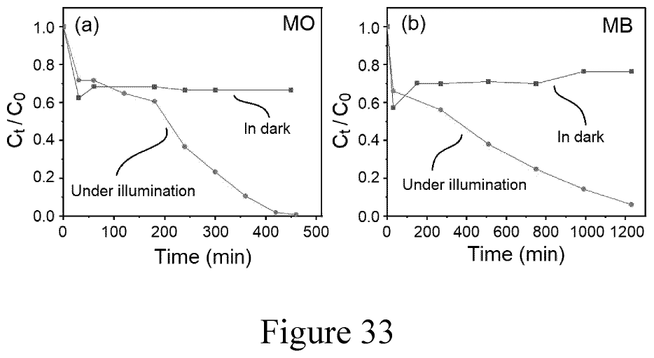

Based on the absorption peak obtained, the concentration of dye solutions could be calculated by comparing the absorption spectrum with that of standard curves. Values of the adsorption capacity can be calculated from equation (12):

$${q}_{e} = \frac{{C}_{0} - {C}_{e}}{m} \times V \qquad ( 12 )$$

where qₑ is the adsorption capacity at equilibrium, m (g) is the mass of adsorbent, and V is the volume of the dye solution. C₀ and Cₑ are the initial and the equilibrium concentration of dye solutions, respectively. The value of organic compound removal performance (either by adsorption or photocatalytic degradation) at a given time (t) can be evaluated based on equation (13):

$$Removal performance ( t ) = \frac{{C}_{t}}{{C}_{0}} \qquad ( 13 )$$

where Cₜ is the concentration of dye in solution at given time (t). The dye removal performances of the product are shown in FIGS. 33a-b. As shown, the adsorption of dye species on the adsorbent reaches the equilibrium after 30 min. The results provided evidence for an adsorption performance between 12-21 mg/g for organic dyes at room temperature, which could increase to 25 mg/g at the higher temperature of 40° C. For the case of MO, the dye was completely removed from the solution after 7 hours of LED illumination (450 nm). This period was 20 hours for MB. Without wishing to be bound by theory, it is believed that the presence of SnO nanocrystals embedded in the organic compounds was responsible for the visible-light photocatalytic performance of the nanocomposite material.

### Example 20—Electrochemical Characterization

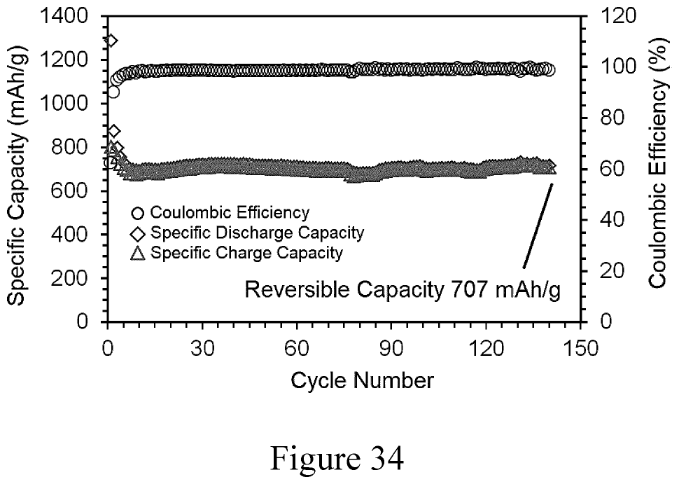

The product of Example 18 was mixed with conductive carbon (C45) and PAA-CMC (1:1, weight ratio) binder with the mass ratio of 7:1:1. The mixture was ground in deionized water to form a uniform slurry, and coated onto copper foil, followed by drying to form an electrode with a mass loading of ≈1.5 mg/cm². The electrode fabricated was assembled into CR2025 half-cell using Li-disc served as the reference/counter electrode, and the LB-010 electrolyte. FIG. 34 shows the Li-ion storage performance of the electrode, demonstrating a reversible capacity of 707 mAh/g after 140 cycles at the current density of 100 mA/g.

The results indicate that the product from Example 18, which contained crystalline organic compounds, SnO and Sn can be employed as the anode of metal-ion batteries, for example the anode of a lithium ion battery. The results also indicate that the organic compounds could effectively influence the electrochemical performance of the metal oxide to maintain its Li-ion storage capacity over at least 140 cycles. Without wishing to be bound by theory, it is believed that the organic compound is able to support maintaining the Li-ion, Na-ion or K-ion storage capacity of the metal oxide or the semimetal oxide over cycling in a battery device

### Example 21—Conversion of PET into Nanostructured Terephthalic Acid Using Inorganic Salts

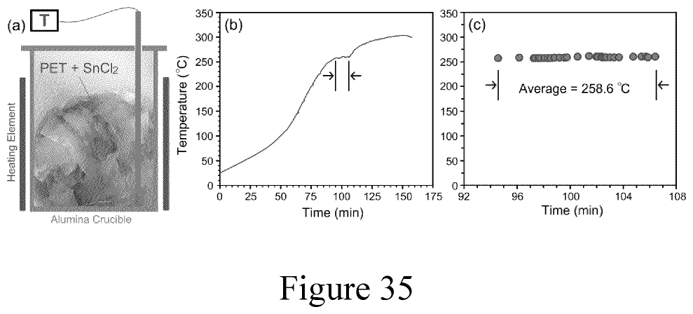

PET mineral water bottles were cut into pieces, and 138.5 g of PET pieces were placed into an alumina crucible. Then, 226.3 g of nominally anhydrous SnCl₂ (Sigma, 208256, reagent grade 98%) was added to the crucible. The crucible was placed in a vertical furnace, and partially covered by an alumina lid. The furnace was heated, while the temperature inside the crucible was recorded using an alumina-shielded thermocouple placed inside the crucible. A schematic of the setup is shown in FIG. 35a. The heating was performed for 151 min to a maximum temperature of 303° C. in air, before the furnace was allowed cool down to room temperature. After the heat-treatment, the contents of the alumina crucible were washed with 700 mL acidic water (HCl-27%). Acidic water was employed to dissolve the SnCl₂ salt without causing hydrolysis (interaction between the SnCl₂ and aqueous solvents to form insoluble oxide phases). Then, the suspension was vacuum filtered and the filtrate was dried at 100° C. for 2 hours.

FIG. 35b shows the temperature-time profile during the heating of the mixture from room temperature to the maximum temperature of 303° C. during 151 min of treatment. The profile shows two sloping lines corresponding to heating the materials from 0 min (26° C.) to 55 min (114° C.), and 55 min to 95 min (257° C.). Then, the profile exhibits a horizontal line from 95 min to 106 min (259° C.). FIG. 35c shows the isotherm event at a greater time resolution. The average temperature in this section was calculated as 258.6° C. Such horizontal temperature-time sections are typically observed during phase transition events, like melting. Thereafter, there is another slopping line, with considerably less slope than those of initial sections, from 106 min to 151 min, where the maximum temperature (303° C.) was recorded. After this point, the furnace was turned-off and the temperature was allowed to reduce to room temperature.

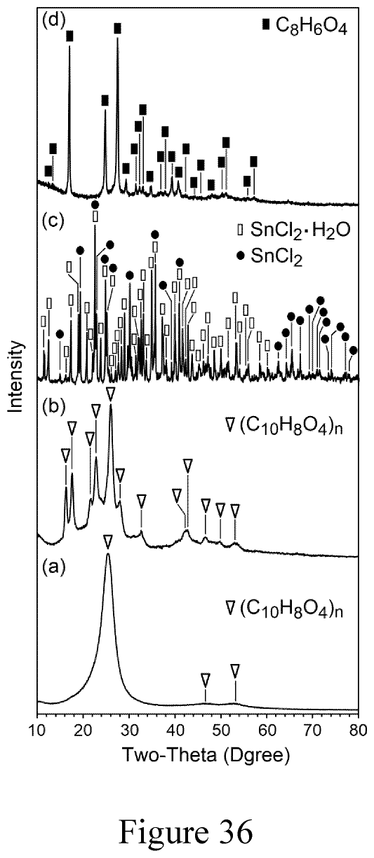

The phase transition that occurred during the heat-treatment process could be observed from the XRD patterns of FIG. 36a-d. FIG. 36a shows the XRD pattern recorded on small pieces of the plastic material, where the broad peak centered at 2θ≈25.4° corresponds to the (100) reflection in semi-amorphous (C₁₀H₈O₄)ₙ polyethylene terephthalate (PET) with disordered anorthic structure. The organic polymer material was heated to 260° C. overnight and then cooled down to room temperature. The XRD pattern of the heat-treated sample obtained is shown in FIG. 36b, indicating the presence of anorthic structured crystalline PET (ICDD≠00-049-2301). The most intense peak observed at 2θ≈26.0 is related to the (100) reflection which has shifted to larger angles in comparison with the original PET, confirming the progress of crystallization.

The XRD pattern of nominally anhydrous SnCl₂ used in this example is shown in FIG. 36c. As observed, the pattern can be characterized by the presence of tin chloride hydrate, SnCl₂·H₂O (ICDD≠01-077-0053) with monoclinic structure as the main phase, and tin chloride, SnCl₂ (ICDD≠01-072-0137) with orthorhombic structure. The quantitative phase analysis conducted on the XRD patterns suggested a proportion of around 59 wt % SnCl₂·H₂O and 41 wt % SnCl₂, indicating the presence of ≈11 wt % hydrated water with this sample. The hydration of the sample may be due to moisture available in the atmosphere during the handling of the sample. The sample obtained by the thermal treatment of nominally anhydrous SnCl₂ (containing hydrated water) and the PET to the maximum temperature of 303° C. is shown in FIG. 36d. As shown, the pattern can be indexed to the terephthalic acid, C₈H₆O₄ (ICDD≠00-022-1941) with anorthic crystalline structure.

### Example 22—Analysis of Thermal Phase Transitions

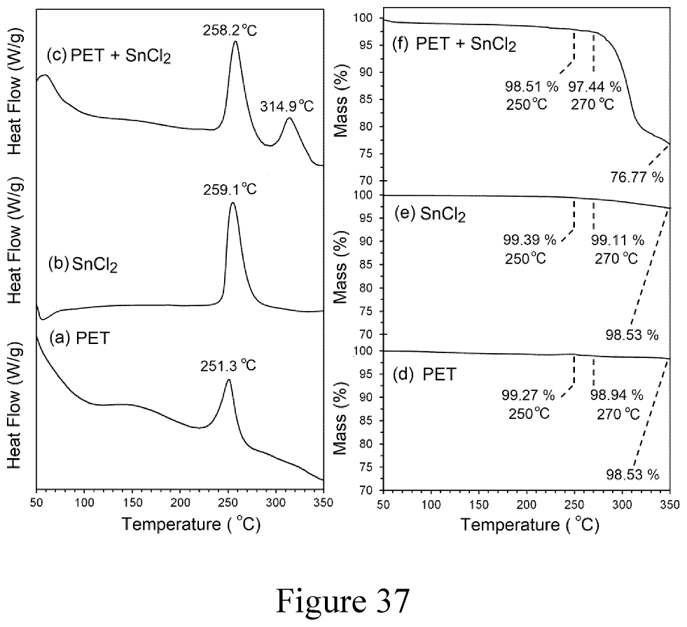

11.046 mg PET and 38.401 mg SnCl₂ were mixed and the mixture was placed in an alumina crucible, which was subsequently employed to measure the DSC and TGA curves of the mixture under air flow of 100 mL min⁻¹ at a heating rate of 10° C. min⁻¹. The results as well as thermograms obtained using PET and SnCl₂ individually recorded under the same conditions are shown in FIGS. 37a-f.

FIG. 37a shows the DSC curve of PET recorded at 50-350° C., in which the endothermic peak observed at 251.3° C. is related to the melting of the material. There is no additional peak, suggesting that the melting event is the sole transition caused by heating PET to 350° C. This is confirmed by the TGA results shown in FIG. 37d, where the total mass loss at 250, 270 and 350° C. were 0.73, 1.06 and 1.47%, respectively. The small mass loss observed was related to the gradual evaporation of surface organic contaminants and moisture. This result was further confirmed by the X-ray diffraction pattern of FIG. 36b, where no phase transition was observed by heating PET overnight at 260° C.

FIG. 37b shows the DSC curve of the nominally anhydrous SnCl₂, which contained around 11 wt. % hydrated water (FIG. 36c) from environmental moisture. The DSC curve only exhibited a noticeable endothermic peak at 259.1° C., related to the melting of SnCl₂. The TGA curve of the sample, shown in FIG. 37e, indicates mass losses of 0.61, 0.89 and 1.47% at 250, 270 and 350° C., respectively. Based on the observations, it can concluded that SnCl₂ is capable of absorbing moisture from the environment and holding a considerable portion of the hydrated moisture at relatively high temperatures, even after the melting event.

FIG. 37c shows the DSC curve recorded on PET (11.046 mg) and SnCl₂ (38.401 mg) mixture. The endothermic peak observed at 258.2° C. can be assigned to the co-melting of PET and SnCl₂, which is in agreement with the isotherm event at 258.6° C. in FIG. 35c. FIG. 37f shows the TGA result from the PET+SnCl₂ mixture, providing evidence for mass losses of 1.49, 2.56 and 23.23% at 250, 270 and 350° C., respectively. Considering the presence of about 11 wt. % hydrated water with the SnCl₂, the total amount of hydrated moisture originating from SnCl₂·H₂O in the salt was estimated as about 8.5 wt. % (4.224 mg). Therefore, a quantity of about 7 wt. % hydrated water was likely present in the mixture of PET+SnCl₂ at 250° C., just before the co-melting event.

Without wishing to be bound by theory, it is believed that the water content could lead to the depolymerisation of PET. According to proposed reaction (3) (see discussion above), the hydrated SnCl₂ and PET co-melt at around 258° C., while the hydrated SnCl₂ retains the majority of its water content. From temperatures of around 290-320° C., the water content of the melt caused the depolymerization of PET into its monomers, namely terephthalic acid (C₈H₆O₄) and ethylene glycol, (CH₂OH)₂, accompanied by the evaporation of the latter together with the remaining moisture. This event was identified in the DSC thermogram of FIG. 37c by the presence of an endothermic peak with maxima at 314.9° C. The 15 wt. % mass loss observed in the temperature range 290-320° C. (FIG. 37f) corresponded to the evaporation of ethylene glycol and remaining moisture leaving solid PET and molten SnCl₂ behind. The XRD pattern of the product obtained by heating of PET+SnCl₂ (FIG. 36d) confirmed the above mechanism. Values for the peaks are shown in Table 3.

TABLE 3
XRD peak information

| Pos. | Height | FWHM | d-spacing | Rel. Int. |
|---|---|---|---|---|
| [°2Th.] | [cts] | [°2Th.] | [Å] | [%] |
| 16.9948 | 3011.02 | 0.1496 | 5.21730 | 98.63 |
| 24.8315 | 1356.01 | 0.1309 | 3.58566 | 44.42 |
| 27.5409 | 3052.97 | 0.1870 | 3.23876 | 100.00 |
| 29.3149 | 286.71 | 0.1122 | 3.04668 | 9.39 |
| 31.4419 | 130.80 | 0.2991 | 2.84526 | 4.28 |
| 32.3090 | 159.47 | 0.1870 | 2.77086 | 5.22 |
| 33.0040 | 134.24 | 0.1870 | 2.71408 | 4.40 |
| 34.8216 | 139.16 | 0.2244 | 2.57646 | 4.56 |
| 36.5462 | 39.82 | 0.8974 | 2.45875 | 1.30 |
| 39.2597 | 372.40 | 0.1309 | 2.29483 | 12.20 |
| 40.6818 | 271.76 | 0.2617 | 2.21784 | 8.90 |
| 48.3471 | 31.43 | 1.1965 | 1.88261 | 1.03 |
| 51.1331 | 81.66 | 0.4487 | 1.78639 | 2.67 |
| 55.8152 | 42.89 | 0.4487 | 1.64712 | 1.40 |
| 57.2170 | 65.79 | 0.5235 | 1.61006 | 2.16 |
| 64.6527 | 20.10 | 0.7296 | 1.44051 | 0.66 |

Values of the full width at half maximum (FWHM, 2-theta) for the peaks at 2-theta values of 16.99, 24.54 and 29.31 are 0.1496, 0.1309 and 0.1870 degrees, respectively. Using these values, the average crystalline sizes were calculated at different crystalline directions based on the Scherer equation:

$$D = ( k λ / βcosθ ) \qquad ( 14 )$$

where k is the Scherer's constant (K=0.9), λ is the X-ray wavelength (1.54 Å), β is FWHM of the diffraction peak in Radian, and θ is the angle of diffraction. Accordingly, the average crystalline sizes for peaks located at 2-theta values of 16.99, 24.54 and 29.31 in the diffraction pattern of FIG. 36d were be calculated as 53.6 nm, 62.1 nm and 43.5 nm, respectively.

### Example 23—Microstructural characterization of waste plastic derived terephthalic acid

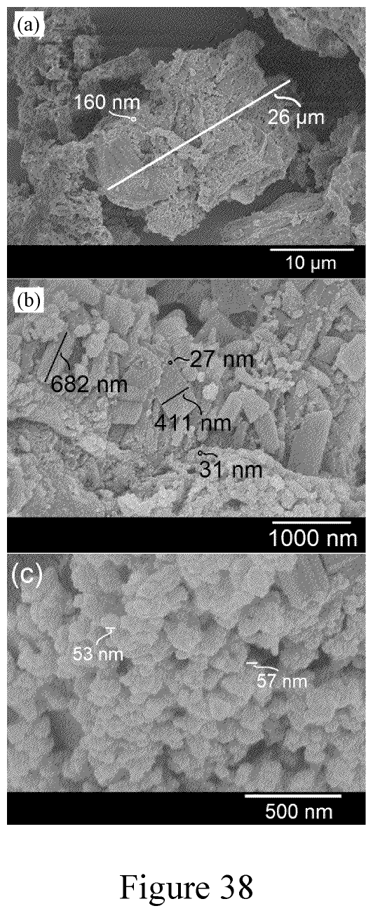

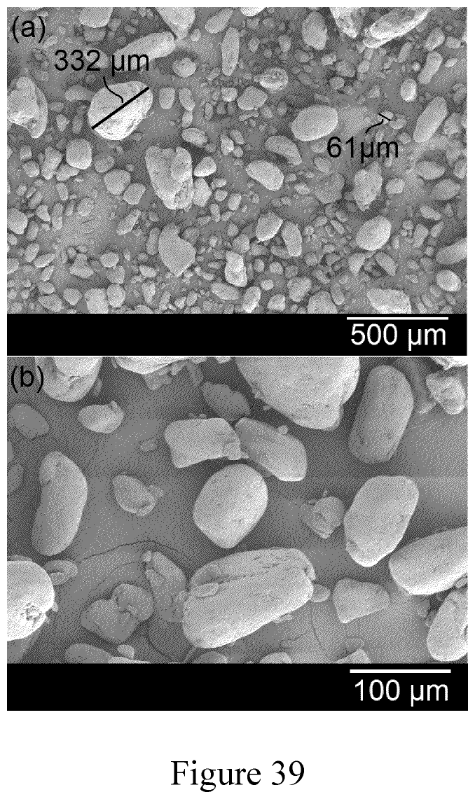

FIGS. 38a-c shows the SEM (FEI Nova Nano-SEM) micrographs of the terephthalic acid made by the heat-treatment of PET and nominally anhydrous SnCl₂ at 303° C. (Example 21). FIG. 38a shows an agglomerated particle with an overall size of 26 μm. This agglomerated particle contains nanometer-sized entities with sizes typically less than 200 nm, as shown in the micrograph. The other morphologies observed in the terephthalic acid product are shown in FIGS. 38b and 38c. FIG. 38b exhibits sheet-like morphologies with overall dimensions typically less than about 1 μm, for instance 411 nm. The sheet-like particles can contain nanometer-sized entities with sizes of less than 50 nm, for instance 27 nm. The presence of agglomerated nanostructured particles is also confirmed in FIG. 38b, where the presence of a nanoparticle with a size of 31 nm within the agglomeration is highlighted. FIG. 38c shows the agglomerated nanoparticles with a higher resolution. From this micrograph, the terephthalic acid nanoparticles were measured to be less than 100 nm, for instance 53 and 57 nm. The microstructure of the produced terephthalic acid was different from available terephthalic acid materials. FIGS. 39a-b shows the SEM micrographs of commercially available crystalline terephthalic acid (Merck, 8.00762, purity≥98%). As can be seen in FIG. 39a, the particles were typically considerably larger than 1 μm, with sizes up to several hundreds of micrometers, for instance 332 μm. FIG. 39b exhibits a larger magnification micrograph confirming that the particles are not porous, unlike the terephthalic acid material produced through the depolymerization of PET with SnCl₂ (FIG. 38a-c).

Terephthalic acid obtained through the depolymerization of PET with SnCl₂ differs from the commercially available terephthalic acid samples, and those reported in the literature due to the unique nanostructured morphology of the former.

### Example 24—Electrochemical Performance of Nanostructured Terephthalic Acid

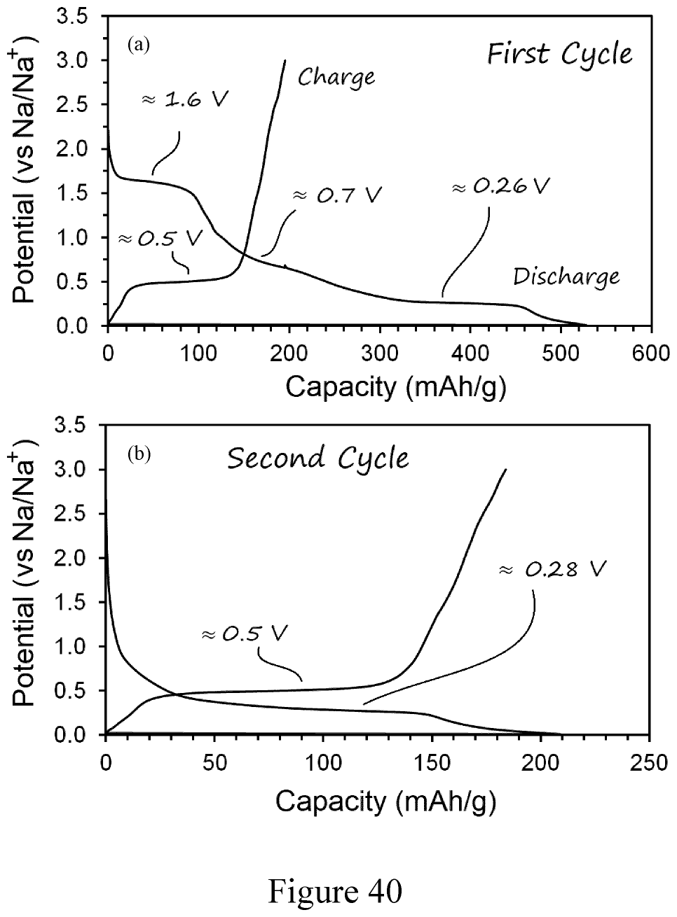

The terephthalate acid produced by depolymerization of waste PET using SnCl₂ (Example 21, as shown in FIG. 38a-c) was uniformly mixed with conductive carbon (C45) and PVDF binder with a mass ratio of 60:30:10 using NMP as the solvent. The resulting slurry was then coated on copper foil using the doctor blade method, and dried in a vacuum oven at 80° C. for 12 h to obtain a mass loading of greater than 1.5 mg/cm². Then, coin-type half-cells (CR 2032) were assembled using metallic sodium as the counter electrode, 1.0 M NaCF₃SO₃ in diglyme as the electrolyte, and glass microfiber (Whatman, 1823025) as the separator. The cells were allowed stabilize at room temperature for 10 hours, and then assembled on a battery test system, where the Na-ion insertion/extraction cycles were conducted at constant current density of 30 mA/g in the voltage range 0.01-3.0 V vs Na/Na⁺. Recorded galvanostatic discharge-charge curves for the first and second cycles are shown in FIGS. 40a-b, respectively. As can be seen, the first cycle can be characterized by the presence of a plateau at around 0.26 V vs Na/Na⁺ (0.23-0.33 V vs Na/Na⁺) in the discharge process and a plateau at around 0.5 V vs Na/Na⁺ (0.45-0.55 V) in the charge process. Likewise, the second cycle can be characterized by the presence of a plateau at around 0.28 V vs Na/Na⁺ (0.25-0.35 V vs Na/Na⁺) in the discharge process, and a plateau at around 0.5 V vs Na/Na⁺ (0.45-0.55 V vs Na/Na⁺) in the charge process. The plateaus observed in the second discharge/charge events were repeated in the subsequent Li-ion insertion/extraction cycles for 100, 500, 1000 and 5000 cycles. The Na-ion storage capacity observed at the second discharge process was recorded at 209.1 mAh/g.

The presence of plateaus at around 0.28/0.5 V vs Na/Na⁺ in the discharge/charge processes is highly beneficial for Na-ion storage technology. These values provide a high-level of safety, avoiding sodium plating on the surface of the electrode that might arise from approaching a voltage of 0 V vs Na/Na⁺. These values are also relatively low, ensuring a high energy density of the battery using the nanostructured terephthalate acid as the anode material.

The results show that Na-ion insertion/extraction into/out of the electrode made of the nanostructured terephthalic acid occurs at voltages of 0.28/0.5 V vs Na/Na⁺, providing both safety and high-energy density characteristics for a battery using the nanostructured terephthalic acid as the anode material. The results suggest that the nanostructured terephthalic acid can be used as the electrode of metal-ion batteries, including the anode of Li-ion, Na-ion and K-ion batteries.

### Example 25—Conversion of PET into Nanostructured Terephthalic Acid with Oxide or Hydroxide Phases using Inorganic Salts

Pieces of PET plastic (20.1 g) made by cutting waste water bottles were mixed with nominally anhydrous SnCl₂ (142.0 g) and the mixture was placed into an alumina crucible. The crucible containing the mixture (162.1 g) was placed in a gas-tight steel retort, and the retort was placed in a vertical resistance furnace. The temperature inside the crucible was measured using an alumina-shielded thermocouple placed inside the mixture. The furnace was heated from room temperature to a maximum temperature of 333° C., while the retort was maintained under flow of Ar gas (60 mL/min). The temperature inside the crucible increased from 23° C. to a maximum temperature of 312° C. over 137 min. Then, the temperature was maintained at the maximum temperature for 13 min before the furnace was turned-off. After cooling the furnace to room temperature, the material inside the crucible was weighed to be 148.3 g. The weight loss (13.8 g) mainly correspond to the evaporation of ethylene glycol and the remaining moisture of the melt, upon conversion of PET into nanostructured terephthalic acid. Conversion of PET into terephthalic acid using hydration (moisture) of the salt, with ethylene glycol removed, can lead to the 13.54% mass loss as shown in reaction (3).

20.1 g PET produced 17.3 g terephthalic acid, leading to a ≈2.8 g mass loss. The additional ≈11 g mass loss was attributed to the evaporation of the remaining hydration moisture during the depolymerization process. Based on these calculations, it is expected that 142 g SnCl₂ can produce 85 g nanostructured terephthalic acid at temperatures around 300° C.

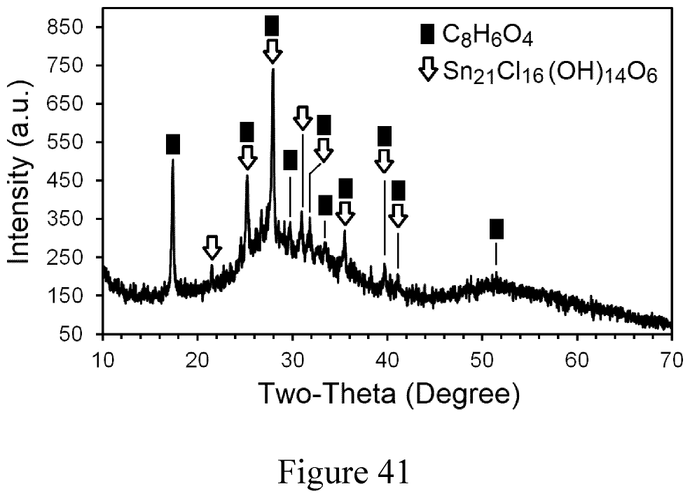

The material obtained by the heat-treatment of PET and SnCl₂ was washed with distilled water (pH≈7), and the suspension obtained was filtered. The filterate was dried at 80° C. for 2 hours, and then subjected to X-ray diffraction analysis. The pattern obtained is shown in FIG. 41, where the presence of terephthalic acid monomer, C₈H₆O₄ (ICDD≠00-021-1919) with anorthic crystalline structure as the main phase and tin oxide chloride hydroxide, Sn₂₁Cl₁₆(OH)₁₄O₆ (ICDD≠00-035-0907), with rhombohedral crystalline structure as the minor phase can be confirmed. The formation of tin oxide chloride hydroxide can be related to the hydrolysis of SnCl₂ during the washing step with distilled water (as shown in reaction (1)).

Tin oxide chloride hydroxide has a relatively low solubility in water, and therefore, remains in the nanostructure of terephthalic acid after washing and filtration.

### Example 26—Heat Treatment of PET with SnCl₂ in the Presence of Si to Fabricate Organic Compound Embedded with SiNPs

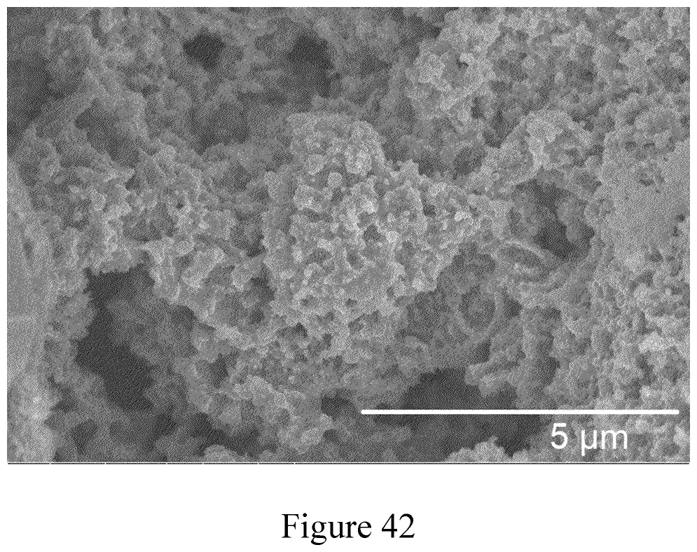

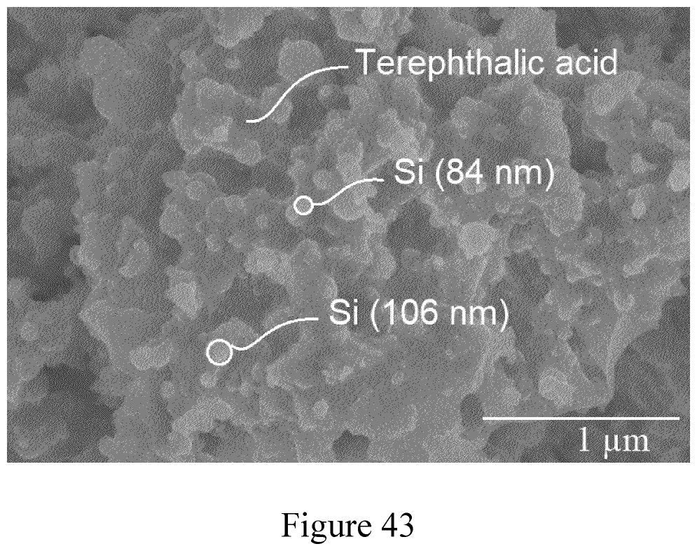

6 g SiNPs (Sigma Aldrich, 633097) with nominal particle sizes of less than 100 nm and spherical morphology was mixed with 20 g waste PET plastic bottles (cut into pieces of few centimes in length) and 159.6 g SnCl₂ powder (Sigma Aldrich, 208256). The mixture was loaded into an alumina crucible. The crucible was placed in an Inconel retort equipped with gas inlet/outlet ports. The retort was located inside a resistance furnace, and heated under flow of Ar-5% H₂ from room temperature to a maximum temperature of 284° C., followed by a dwell time of 5 min. Then, the temperature was reduced to room temperature, and 1 g of the product obtained was washed with diluted HCl acid (10%) to remove the remaining salt, followed by drying at 100° C. for 2 h under flow of Ar-5% H₂. Then, the sample obtained was measured using electron microscopy. FIG. 42 shows SEM micrograph of the product demonstrating the presence of a porous morphology in which SiNPs were embedded within terephthalic acid. FIG. 43 exhibits a higher magnification SEM micrograph demonstrating the presence of spherical SiNPs within the terephthalic acid matrix. Two SiNPs with diameters of 84 and 106 nm are highlighted in the micrograph.

### Example 27—Electrochemical Performance of Organic Compound Embedded with SiNPs

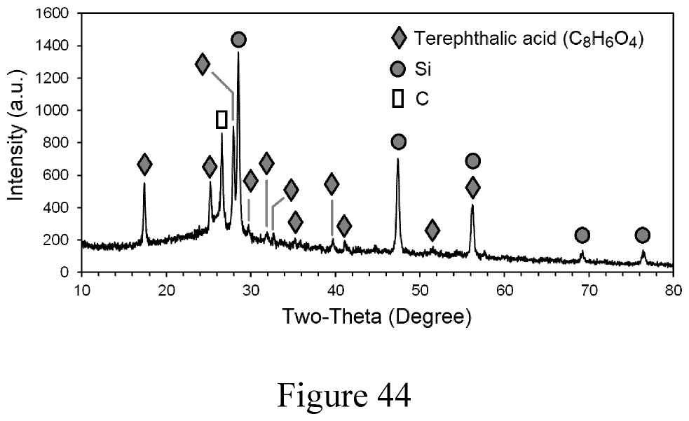

12.21 g of the product obtained by heating the mixture of PET, SnCl₂ and SiNPs to 284° C. (Example 26) together with 2.05 graphene nanosheets were added into 400 mL concentrated sulfuric acid, and the suspension was subjected to ultrasonication for 20 min, followed by 20 min magnetic stirring. The suspension was filtered, and the filtrate was washed with deionized water and dried at 180° C. under flow of Ar-5% H₂ for 5 h. The XRD pattern of the product obtained is shown in FIG. 44. As can be observed, the product obtained contained terephthalic acid (ICCD: 00-021-1919), elemental Si (ICCD: 01-089-2749) and graphitic carbon (ICCD: 01-075-2078). EDS analysis performed on the sample revealed the presence of Si (˜28 wt. %) in the sample.

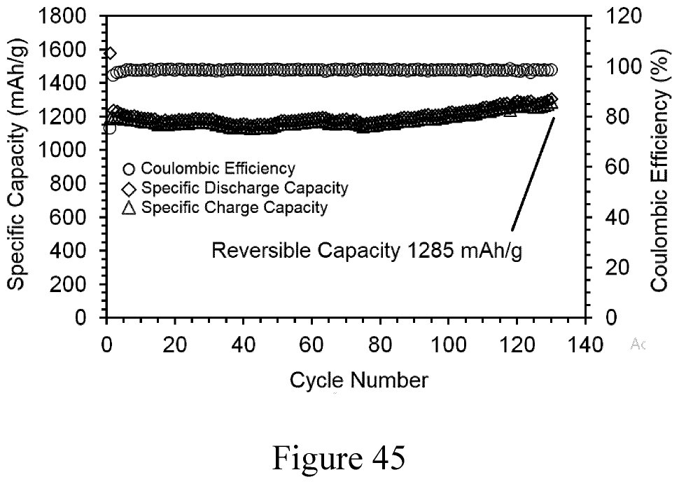

The material obtained was used to prepare a lithium ion battery anode. PI powder was added into 400 μL NMP and stirred. Then, the sample containing Si, terephthalic acid and graphene (FIG. 44) was added into the PI solution, and the mixture was subject to ultrasonication to obtain a uniform slurry followed by 10 hours of magnetic stirring. The resulting uniform slurry was coated on Cu foil, and dried at room temperature for 10 min, and then at 100° C. for 2 hours under vacuum to remove NMP. After drying, the electrode was heated to 250° C. with a heating rate of 2° C./min under flow of Ar-4% H₂ in a tube furnace, with a dwell time at 250° C. of 2 h. The temperature was then reduced to room temperature under the same gas flow. The electrode obtained was used to assemble coin cell CR 2025 half-cells in which metallic Li was used as both the counter and reference electrode. 1.0 M LiPF₆ in EC:DEC:EMC (1:1:1 wt. %) and Celgard 2400 polypropylene films were used as the electrolyte and separator, respectively. The cells were allowed to equilibrate for 10 h at room temperature, and then galvanostatically tested at 0.01-3.0 V vs Li/Li⁺ under a constant current density of 100 mA/g. Typical cycling performance of the electrode is shown in FIG. 45. As can be observed, a reversible capacity of 1285 mAh/g could be recorded after 130 Li-ion insertion and extraction cycles. The capacity was measured based on the total mass of the Si/terephthalic acid/graphene.

The results show that the material with SiNPs embedded within the crystalline organic compound can be used as the anode of metal-ion batteries, such as the anode of lithium-ion batteries.

### Example 28—Conversion of Waste Plastics into Zinc Metal-Organic Frameworks Embedded in Organic Compounds

16.1 g ZnCl₂ (Sigma, 98%) was mixed with small pieces of waste PET material (11.5 g). The mixture (27.6 g) was placed into an alumina crucible, and the crucible was placed inside a gas-tight steel retort. The latter was placed inside a vertical furnace. The retort was heated while a flow of Ar gas (60 mL/min) was directed through the retort. The temperature inside the crucible (measured using an alumina-shielded thermocouple) was increased from room temperature to 373° C. by a heating rate of 7° C./min. Then, the furnace was turned-off and the temperature was allowed to cool-down to room temperature. The product obtained (21.4 g) was washed with distilled water to dissolve the salt, followed by filtration. The filtrate obtained was dried at 80° C. for 2 h, and then characterized.

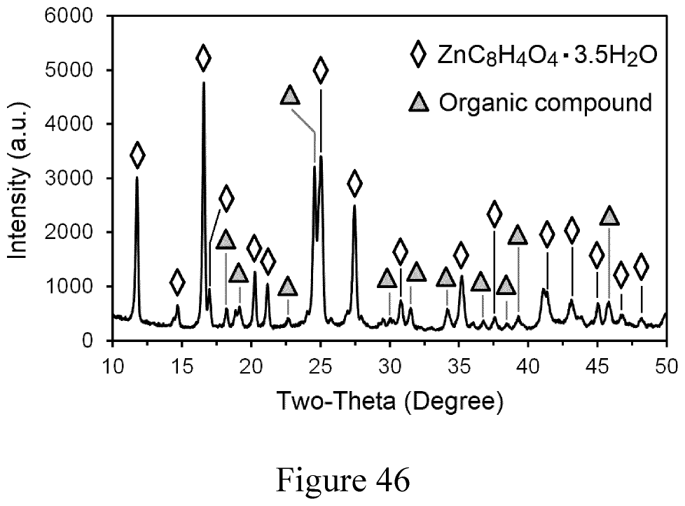

FIG. 46 shows the X-ray diffraction pattern of the product, based on which the presence of zinc terephthalate hydrate (ZnC₈H₄O₄·3.5H₂O, or ZnTP·3.5H₂O, ICDD: 00-038-1777) as the main phase was confirmed. FIG. 46 shows that in addition to zinc metal-organic frame work, there are also XRD peaks related to an organic compound with general formula of CₓH_{y}O_{z}. The two-theta degree, FWHM and intensity values associated with (ZnC₈H₄O₄·3.5H₂O and the observed organic compound are reported in Table 4.

TABLE 4
XRD peak information, indicating the ZnC₈H₄O₄•3.5H₂O
(herein called A) and the organic compound (herein called B)
in the two-theta 1-50 degree.

| Pos. | Height | FWHM Left | d-spacing | Rel. Int. | |
|---|---|---|---|---|---|
| [°2Th.] | [cts] | [°2Th.] | [Å] | [%] | Phase |
| 9.482808 | 146.116600 | 0.112176 | 9.32675 | 3.33 | B |
| 11.751230 | 2650.078000 | 0.149568 | 7.53095 | 60.39 | A/B |
| 14.736880 | 340.517500 | 0.130872 | 6.01124 | 7.76 | A/B |
| 16.592320 | 4388.416000 | 0.186960 | 5.34297 | 100.00 | A/B |
| 16.987260 | 651.004000 | 0.112176 | 5.21963 | 14.83 | A/B |
| 18.230590 | 323.419600 | 0.149568 | 4.86636 | 7.37 | B |
| 18.860090 | 292.950500 | 0.112176 | 4.70533 | 6.68 | B |
| 19.148790 | 362.369500 | 0.112176 | 4.63504 | 8.26 | B |
| 20.269260 | 1003.419000 | 0.168264 | 4.38128 | 22.87 | B |
| 21.162970 | 788.494300 | 0.149568 | 4.19823 | 17.97 | B |
| 22.708300 | 135.086900 | 0.186960 | 3.91591 | 3.08 | B |
| 24.054380 | 282.649900 | 0.112176 | 3.69974 | 6.44 | A/B |
| 24.571930 | 2962.649000 | 0.168264 | 3.62298 | 67.51 | A/B |
| 25.088680 | 2969.713000 | 0.186960 | 3.54951 | 67.67 | B |
| 25.774590 | 160.098400 | 0.112176 | 3.45659 | 3.65 | B |
| 26.944970 | 291.683300 | 0.224352 | 3.30905 | 6.65 | B |
| 27.463230 | 2248.561000 | 0.261744 | 3.24777 | 51.24 | A/B |
| 27.968800 | 190.193600 | 0.149568 | 3.19020 | 4.33 | A/B |
| 29.549270 | 147.371100 | 0.149568 | 3.02307 | 3.36 | B |
| 30.018930 | 171.266600 | 0.149568 | 2.97684 | 3.90 | B |
| 30.793560 | 504.628800 | 0.149568 | 2.90370 | 11.50 | B |
| 31.532050 | 369.745100 | 0.186960 | 2.83736 | 8.43 | B |
| 32.206890 | 34.180160 | 0.224352 | 2.77943 | 0.78 | B |
| 34.174190 | 356.208200 | 0.261744 | 2.62380 | 8.12 | B |
| 35.202210 | 993.416100 | 0.186960 | 2.54949 | 22.64 | A/B |
| 36.032150 | 121.249400 | 0.186960 | 2.49265 | 2.76 | B |
| 36.746310 | 145.452400 | 0.186960 | 2.44583 | 3.31 | B |
| 37.595320 | 209.144000 | 0.186960 | 2.39253 | 4.77 | B |
| 38.528160 | 80.418400 | 0.224352 | 2.33672 | 1.83 | B |
| 39.279810 | 205.299900 | 0.149568 | 2.29372 | 4.68 | B |
| 41.060180 | 663.329000 | 0.149568 | 2.19829 | 15.12 | B |
| 41.378000 | 581.749900 | 0.112176 | 2.18213 | 13.26 | A/B |
| 43.869840 | 72.456390 | 0.149568 | 2.06379 | 1.65 | B |
| 44.999370 | 339.056400 | 0.112176 | 2.01458 | 7.73 | A/B |
| 48.160850 | 143.961500 | 0.261744 | 1.88947 | 3.28 | B |

### Example 29—Conversion of Waste Plastics into Metal-Organic Frameworks

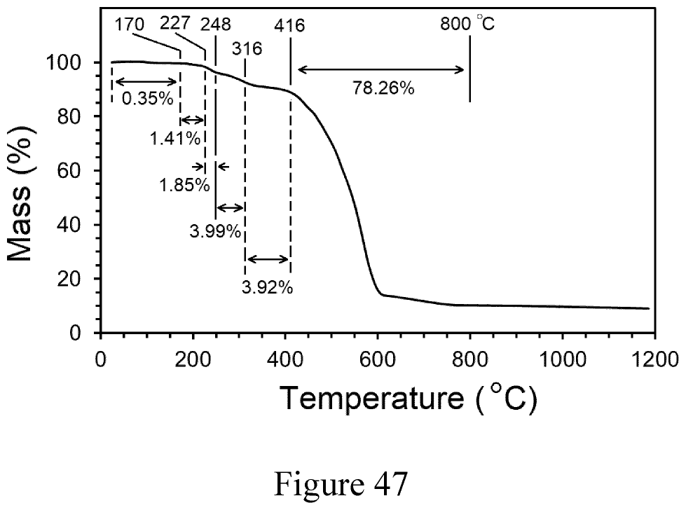

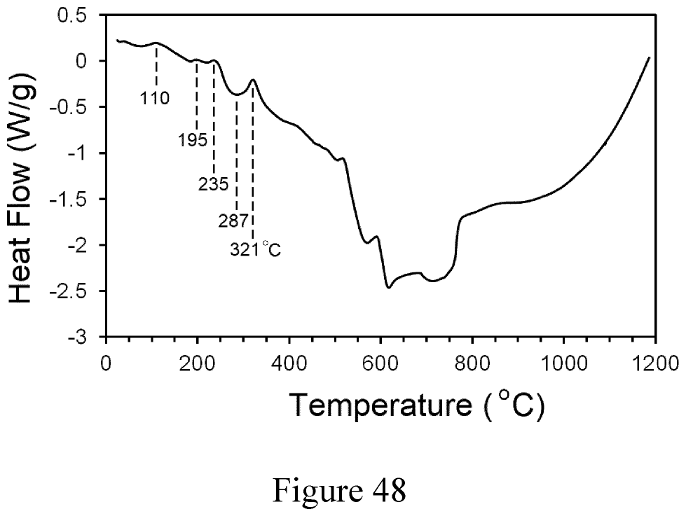

8.47 mg PET pieces were mixed with 30.51 g nominally dry ZnCl₂ and the mixture (38.98 mg) was placed in an alumina crucible. The crucible was placed in a thermal analysis aperture, and heated at a heating rate of 20° C./min to 1200° C. under Ar flow of 100 mL/min. FIGS. 47 and 48 show the recorded TGA and DSC curves, respectively.

According to the TGA curve of FIG. 47, there is a mass loss of 0.35% upon heating to 170° C., which is accompanied by an endothermic event with a peak at 110° C. (FIG. 48). This event is related to the evaporation of non-hydrated moisture of the material. The second endothermic peak is observed at 195° C. which is related to the glass transition event. This event is accompanied by a further evaporation of moisture (1.41%) at the temperature window of 170-227° C. The third endothermic peak (235° C., FIG. 48) is accompanied by a mass loss of 1.85% in the temperature window of 227-248° C. This event can be related to the evaporation of hydrated water of ZnCl₂. The dehydration of ZnCl₂ provides moisture for the depolymerization of PET. This event can be related to an exothermic event with a peak at 287° C., which is accompanied by the evaporation of the by product of the depolymerization (e.g, ethylene glycol) and the remaining moisture leading to a mass loss of 3.99 wt % in the temperature window of 248-316° C. Without wishing to be bound by theory, based on the DSC curve of FIG. 48, there is an endothermic event at 321° C. which corresponds to the melting of dehydrated ZnCl₂, and the reaction (e.g., instant reaction) of melted ZnCl₂ melt with monomers obtained by the depolymerization of PET (such as terephthalic acid, TPA) to form zinc terephthalate (see FIG. 5c).

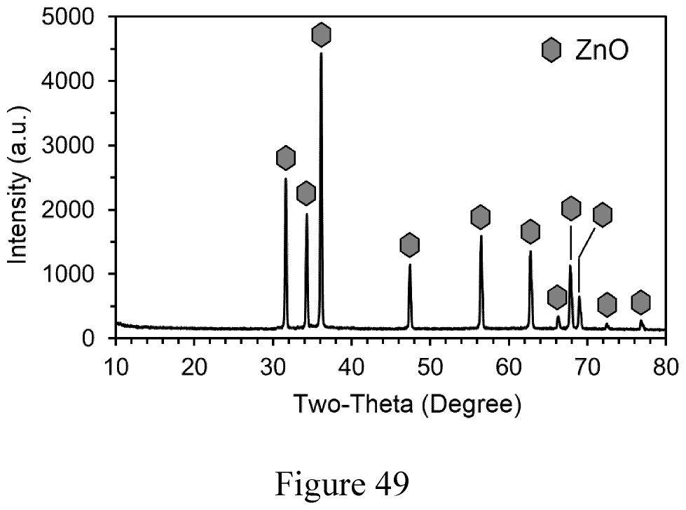

As can be realized from the TGA graph of FIG. 47, a mass loss of 3.92 wt % is observed within 316-416° C., which can be related to the gradual removal of HCl from the system. From 416 to ˜800° C., a major mass loss of 78.26% occurred which can be related to the decomposition of the zinc metal-organic framework, the remaining organic matter (terephthalic acid+terephthalate) and the evaporation of molten ZnCl₂. The remaining mass was relatively stable at greater temperatures and a quantity of 9.0 g was obtained at the end of the thermal treatment at 1200° C. The remaining material was analyzed by XRD (FIG. 49) and found to be ZnO with hexagonal crystalline structure (ICSD: 01-079-0207).

### Example 30—Morphological Characterization of Zinc Metal-Organic Framework Embedded in Organic Compound

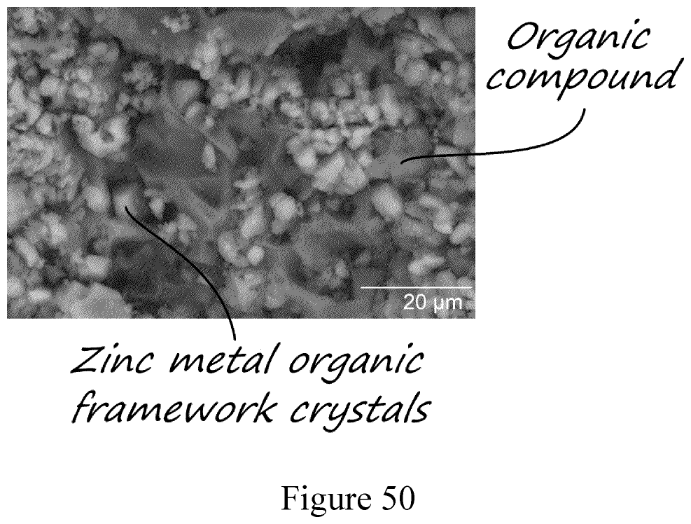

FIG. 50 shows a backscattered electron micrograph of the product of Example 28 demonstrating the presence of crystals with dimensions of substantially less than 1 μm to several micrometers, embedded in the organic compound.

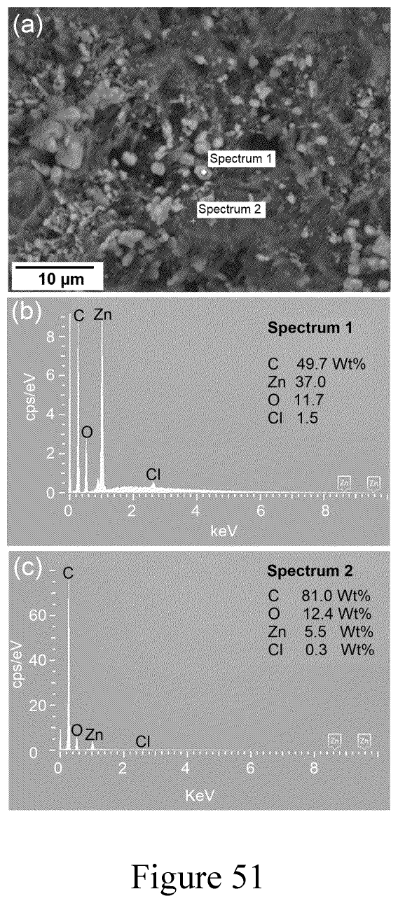

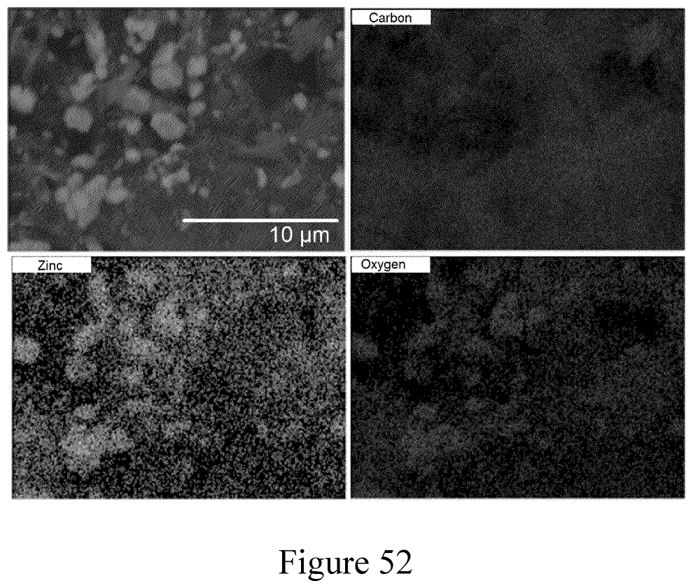

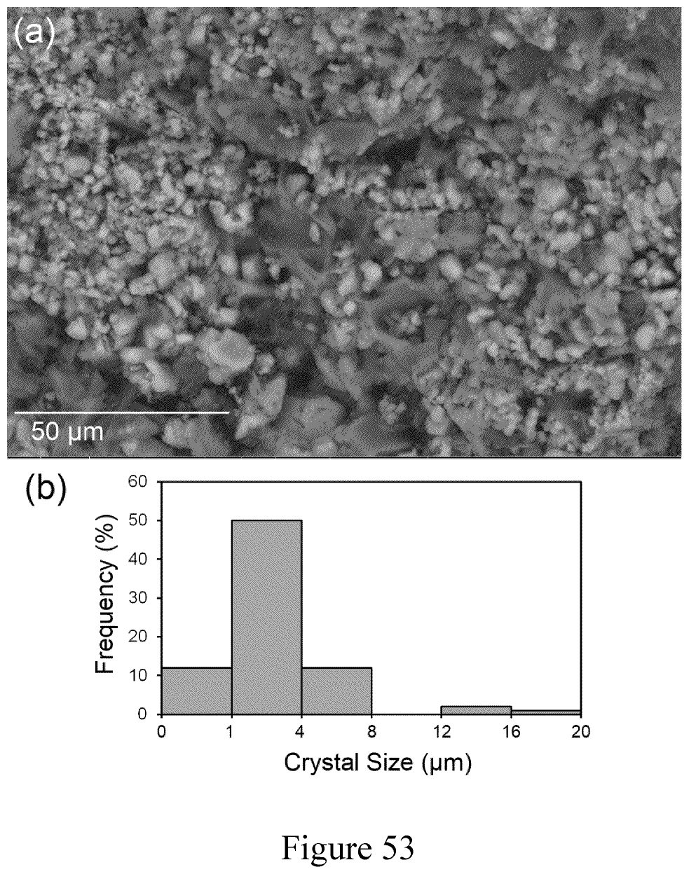

The energy dispersive X-ray spectra (EDS) recorded on the zinc metal-organic framework crystal and the organic compound are shown in FIG. 51a-c, confirming the presence of the dominant elements of C, Zn, and O in the zinc metal-organic framework, and C and O in organic compound. Not that hydrogen is not detectable through EDS. The presence of small amounts of Cl in the sample is evident, which can be residual of the ZnCl₂ used during the preparation of the product. Furthermore, the EDS elemental map analysis recorded on backscattered electron micrograph is shown in FIG. 52. The much greater concentration of zinc in the zinc metal-organic framework crystals was evident. The zinc metal-organic crystals have various sizes and shapes. A histogram of size distribution of the crystals was generated by counting about 100 crystals, as shown in FIG. 53b. As can be seen, the crystals typically have dimensions less than 20 μm, while majority of them have dimensions between 1-4 μm. There is also a fraction of crystals in the range of about 10 nm to 1 μm.

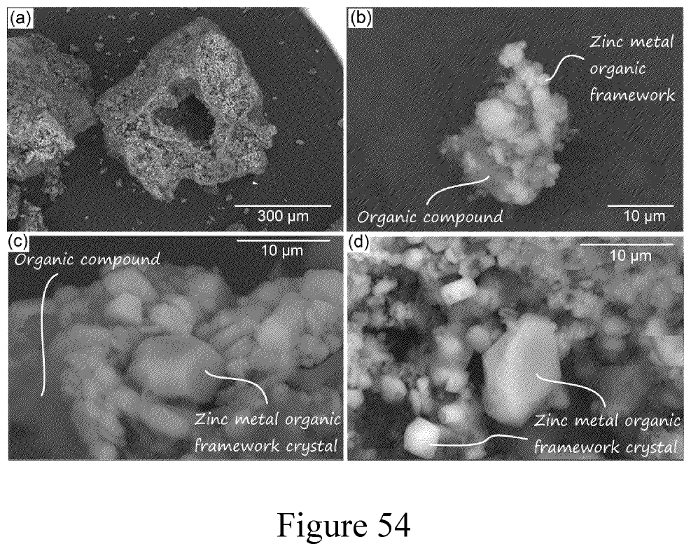

FIGS. 54a-d show backscattered electron micrographs of zinc metal-organic framework embedded in organic compound (product of Example 28), highlighting some morphological features of the nanocrystals. As can be observed from FIG. 54a, the overall size of the zinc metal-organic framework embedded in organic compound particles was at most several hundreds of micrometers, for example 700 μm, 500 μm and 400 μm. According to FIG. 54b, the overall size of the zinc metal-organic framework embedded in organic compound particles was at least 50 μm, 40, μm, 30 μm, 20 μm, 10 μm, 8 μm, 5 μm, 2 μm, 1 μm or 500 nm. According to FIGS. 54c and 54d, zinc metal-organic framework crystals were faceted crystals, and these crystals were embedded within the organic compound. The faceted zinc metal-organic framework crystals had sizes in the range of 50 nm to 10 μm.

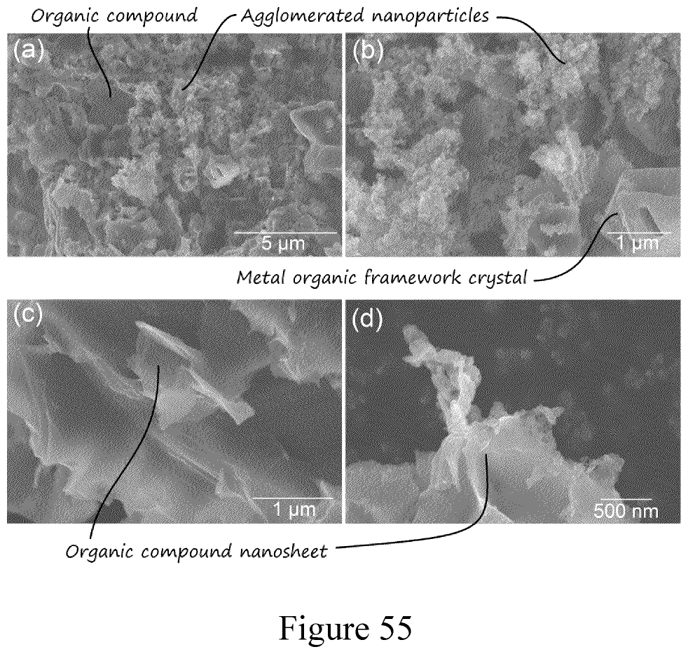

FIGS. 55a-d show secondary electron scanning electron micrographs of zinc metal-organic framework embedded in organic compound (product of Example 28), highlighting some morphological features of the organic compound. According to FIGS. 55a and 55b, the product contained particles with overall dimensions as large as 600 μm or as small as 10 μm. These particles contained zinc metal-organic crystals embedded in organic compound. The shapes of zinc metal-organic framework crystals are highlighted in FIGS. 55c and 55d. These figures show the presence of faceted crystals with square, rectangular, pentagonal or hexagonal surfaces. Although the majority of crystals were embedded in the organic compound, separated zinc metal-organic framework crystals were also be detected.

Based on the micrographs of FIGS. 55a-d, apart from metal-organic framework crystals, two morphologies were observed: sheet-like particles and agglomerated nanoparticles. The organic compound sheet-like particles can be observed in FIGS. 55c and 55d. The sheet-like particles have lateral dimensions from 100 nm to several micrometers, 10 μm for instance. The thickness of the sheet-like particles was assumed to be between 1-10 nm.

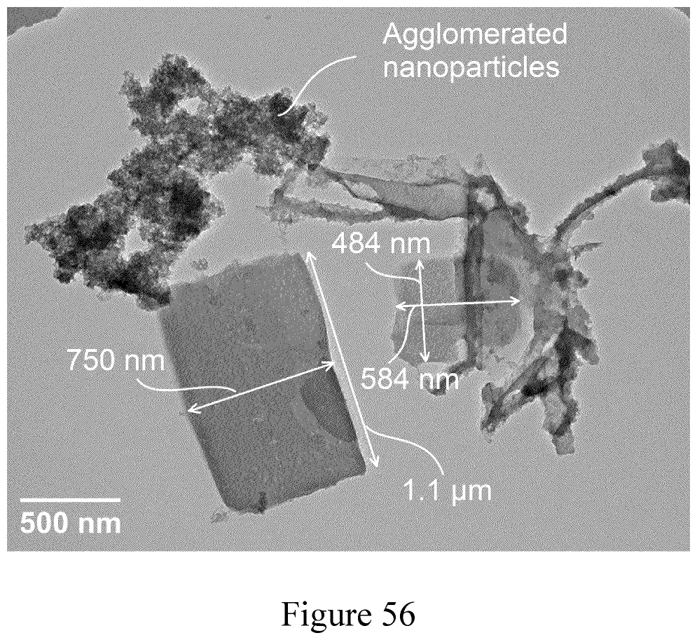

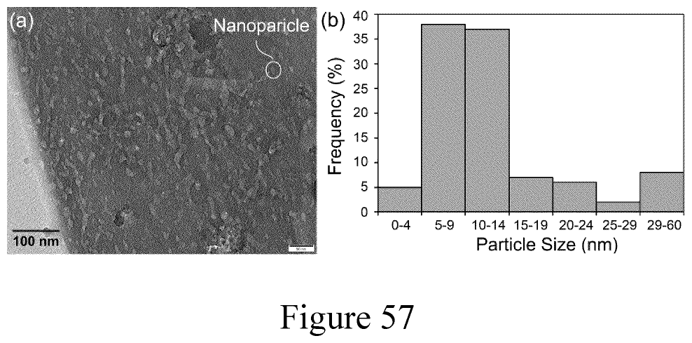

Further insights into the morphological characteristics of the zinc metal-organic framework embedded in organic compound product were be obtained by transmission electron microscopy (TEM, Tecnai F20, 200 kV). FIG. 56 exhibits a low-magnification TEM micrograph of the product, characterized as zinc terephthalate hydrate (ZnC₈H₄O₄·XH₂O) embedded in organic compound. The presence of textured sheet-like particles and agglomerated nanoparticles is evident. Two textured sheet-like particles with dimensions of 750 nm×1.1 μm and 484 nm×584 nm can be observed in FIG. 56. The surface of the sheet-like particles is textured/decorated with nanoparticles, as can be seen in high magnification TEM micrograph of FIG. 57a, recorded on a sheet-like particle.

As can be seen in FIG. 57a, the surfaces of sheet-like particles are patterned with nanoparticles of various sizes and shapes, mainly semi-spherical and worm-like nanoparticles. FIG. 57b shows a histogram of nanoparticle size distribution, obtained by recording the dimensions of more than 100 nanoparticles present of the surface of the sheet-like particle of FIG. 57a. Surface nanoparticles had dimensions in the range 0-4 nm (5%), 5-9 nm (38%), 10-14 nm (37%), 15-19 nm (7%), 20-24 nm (6%), 25-29 nm (2%) and 30-60 nm (8%).

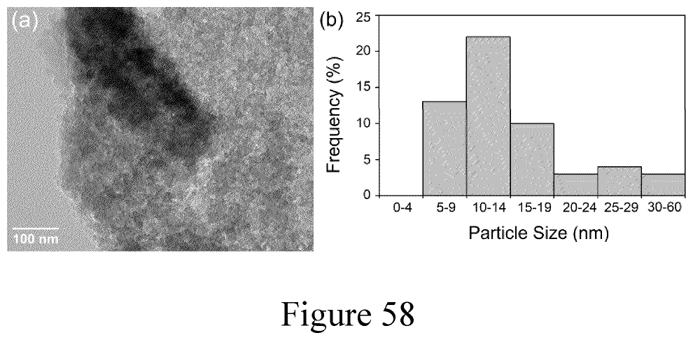

FIG. 58a shows a TEM micrograph recorded on prepared zinc metal-organic framework embedded in organic compound, showing a micrometer-sized particle containing agglomerated nanoparticles. As can be observed, nanoparticles of various sizes are agglomerated leading to the formation of the micrometer-sized particle. FIG. 58b shows a histogram of nanoparticles size distribution, obtained by recording the dimensions of more than 60 nanoparticles present in the micrograph of FIG. 58a. Nanoparticles had dimensions in the ranges 5-9 nm (38%), 10-14 nm (22%), 15-19 nm (10%), 20-24 nm (3%), 25-29 nm (4%) and 30-60 nm (3%).

### Example 31—Li-Ion Storage Performance of Zinc Metal-Organic Framework Embedded in Organic Compound

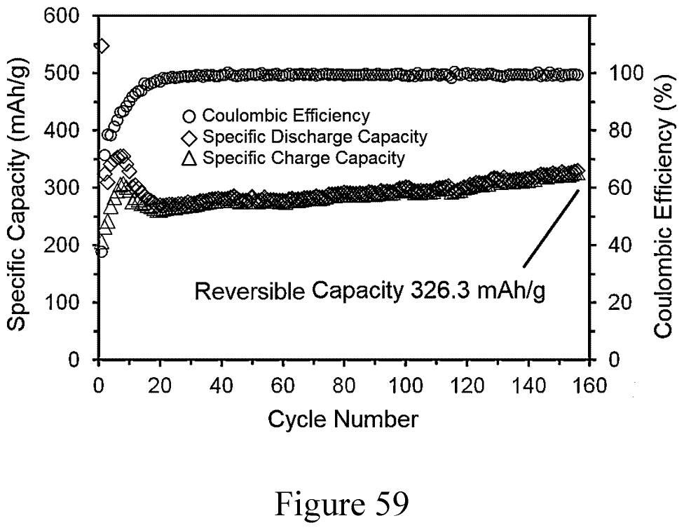

ZnC₈H₄O₄·3.5H₂O embedded in C_{X}H_{y}O_{z} (as described in Examples 28 and 30) was used to make an electrode. ZnC₈H₄O₄·3.5H₂O embedded in C_{X}H_{y}O_{z}, PVDF binder (in NMP) and conductive carbon (Super P) (6:3:1) were thoroughly mixed to make a uniform slurry, which was then coated on copper foil, and dried at 80° C. for 10 h under vacuum to remove NMP. The electrode obtained with a mass loading of greater than 1.5 mg/cm² was used to assemble a CR 2025 coin cell, employing metallic Li as both the counter and reference electrode, and 1.0 M LiPF₆ in EC:DEC:EMC (1:1:1 wt %) and Celgard 2400 polypropylene films as the electrolyte and separator, respectively. The cycling performance was measured at the current density of 100 mA/g in potential range 0.01-3 V vs Li/Li⁺. The results obtained are shown in FIG. 59. A reversible specific capacity of 326.3 mAh/g could be recorded after 156 Li-ion insertion and extraction cycles, demonstrating an impressive capacity.

### Example 32—Heat Treatment of ZnC₈H₄O₄·3.5H₂O Embedded in Organic Compound

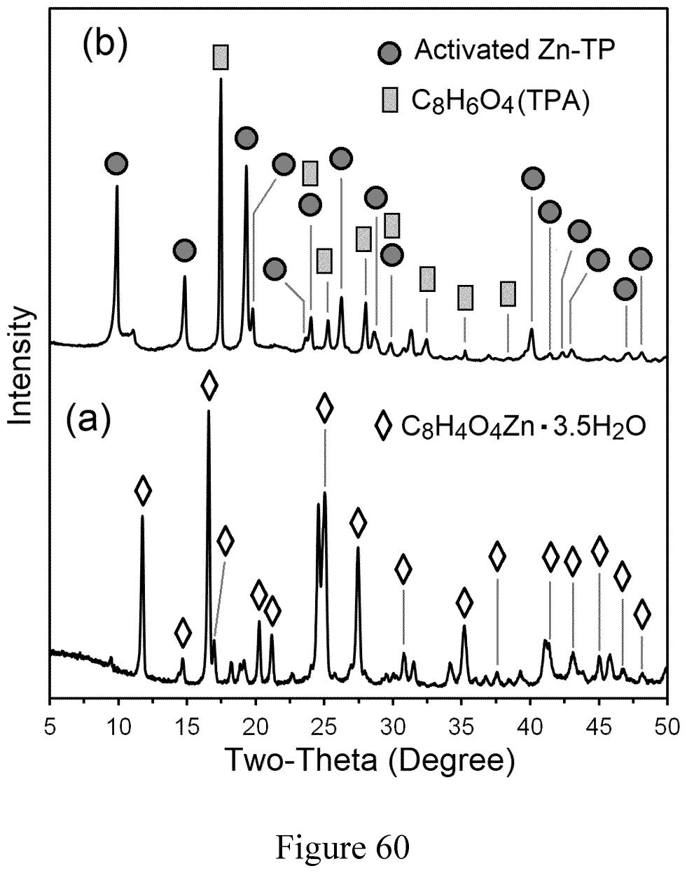

ZnC₈H₄O₄·3.5H₂O embedded in organic compound (C_{X}H_{y}O_{z}) was be transformed into heat-treated zinc terephthalate (Zn-TP) embedded in terephthalic acid (TPA) by a heat-treatment process. ZnC₈H₄O₄·3.5H₂O embedded in organic compound (C_{X}H_{y}O_{z}) was heated at 150° C. for 2 hours under Ar. X-ray diffraction pattern of the heat-treated sample, in comparison with that of the original sample is shown in FIGS. 60a-b. The XRD original sample is shown in FIG. 60a and the XRD heat-treated sample is shown in FIG. 60b. The heat-treated material is characterized to contain heat-treated zinc terephthalate (Zn-TP) and terephthalic acid (TPA). The heat-treated Zn-TP was indexed based on the simulated XRD pattern of the single crystal compound [M. Nakhaei et al., Antibacterial activity of three zinc-terephthalate MOFs and its relation to their structural features, Inorganica Chimica Acta 522 (2021) 120353].

In FIG. 60a, the diffraction peak at 2-theta value of 11.75° had a FWHM of 0.150° and the crystalline domain size of 53.2 nm. The diffraction peak at 2-theta value of 16.5920 had a FWHM of 0.187° and a crystalline domain size of 42.9 nm. The diffraction peak at 2-theta value of 35.2020 had a FWHM of 0.228° and a crystalline domain size of 36.5 nm.

In FIG. 60b, the FWHM (2-theta, degree) values for peaks at 9.89, 19.33, 25.27 and 40.11 degree are 0.1299, 0.1948, 0.1624 and 0.2922, respectively. The average crystalline domain sizes associated to these peaks were calculated to be 61.4 nm, 41.3 nm, 50.1 nm and 28.9 nm. Values for the peaks are shown in Table 5.

TABLE 5
XRD peak information

| Pos. | Height | FWHM | d-spacing | Rel. Int. |
|---|---|---|---|---|
| [°2Th.] | [cts] | [°2Th.] | [Å] | [%] |
| 9.8864 | 13729.89 | 0.1299 | 8.94689 | 60.97 |
| 11.0894 | 1415.97 | 0.1624 | 7.97887 | 6.29 |
| 14.8318 | 5953.82 | 0.1624 | 5.97297 | 26.44 |
| 17.4498 | 22520.21 | 0.1624 | 5.08230 | 100.00 |
| 19.3259 | 15308.36 | 0.1948 | 4.59296 | 67.98 |
| 19.7846 | 3602.42 | 0.1299 | 4.48749 | 16.00 |
| 21.4834 | 597.91 | 0.3897 | 4.13633 | 2.65 |
| 23.6231 | 1319.29 | 0.1624 | 3.76630 | 5.86 |
| 24.0256 | 3054.31 | 0.1624 | 3.70410 | 13.56 |
| 25.2705 | 2852.44 | 0.1624 | 3.52439 | 12.67 |
| 26.2512 | 4809.08 | 0.2273 | 3.39491 | 21.35 |
| 28.0173 | 4416.08 | 0.1948 | 3.18479 | 19.61 |
| 28.6306 | 1987.39 | 0.2273 | 3.11794 | 8.82 |
| 29.8468 | 1138.66 | 0.2273 | 2.99362 | 5.06 |
| 30.7975 | 788.42 | 0.1624 | 2.90334 | 3.50 |
| 31.3231 | 2301.06 | 0.2598 | 2.85581 | 10.22 |
| 32.4478 | 1582.85 | 0.1624 | 2.75934 | 7.03 |
| 33.4543 | 217.96 | 0.1624 | 2.67860 | 0.97 |
| 34.5762 | 279.46 | 0.2273 | 2.59420 | 1.24 |
| 35.2634 | 711.20 | 0.0974 | 2.54521 | 3.16 |
| 36.9912 | 429.07 | 0.1948 | 2.43020 | 1.91 |
| 38.4714 | 181.42 | 0.3897 | 2.34004 | 0.81 |
| 40.1129 | 2649.52 | 0.2922 | 2.24798 | 11.77 |
| 41.4170 | 630.40 | 0.1624 | 2.18017 | 2.80 |
| 42.3843 | 732.05 | 0.1948 | 2.13262 | 3.25 |
| 42.9927 | 972.31 | 0.1948 | 2.10385 | 4.32 |
| 45.3727 | 418.25 | 0.2273 | 1.99887 | 1.86 |
| 46.0717 | 210.05 | 0.1948 | 1.97016 | 0.93 |
| 47.2000 | 714.43 | 0.1948 | 1.92566 | 3.17 |
| 48.1390 | 789.32 | 0.2273 | 1.89027 | 3.50 |
| 49.1294 | 227.60 | 0.1299 | 1.85446 | 1.01 |
| 50.0380 | 425.32 | 0.5196 | 1.82290 | 1.89 |
| 50.7185 | 383.78 | 0.0974 | 1.80002 | 1.70 |
| 51.5949 | 447.53 | 0.1299 | 1.77149 | 1.99 |
| 53.2626 | 191.67 | 0.2598 | 1.71989 | 0.85 |
| 55.7023 | 225.11 | 0.1948 | 1.65021 | 1.00 |
| 56.7651 | 272.43 | 0.1624 | 1.62181 | 1.21 |
| 57.7324 | 194.05 | 0.2598 | 1.59692 | 0.86 |
| 59.4284 | 177.27 | 0.1948 | 1.55533 | 0.79 |
| 60.1083 | 232.57 | 0.1948 | 1.53936 | 1.03 |
| 60.7418 | 165.12 | 0.3897 | 1.52481 | 0.73 |
| 61.8632 | 186.59 | 0.3247 | 1.49983 | 0.83 |
| 62.7501 | 87.62 | 0.3247 | 1.48076 | 0.39 |
| 63.5379 | 139.52 | 0.3247 | 1.46428 | 0.62 |
| 68.2510 | 164.90 | 0.3247 | 1.37420 | 0.73 |
| 70.2445 | 168.44 | 0.3247 | 1.34001 | 0.75 |
| 70.8199 | 73.07 | 1.0391 | 1.33053 | 0.32 |
| 73.1061 | 42.28 | 0.3897 | 1.29446 | 0.19 |
| 74.3049 | 96.87 | 0.6494 | 1.27652 | 0.43 |
| 78.1805 | 70.90 | 0.4752 | 1.22164 | 0.31 |

### Example 33—Metal-Ion Storage Performance of Heat-Treated Zinc Terephthalate (Zn-TP) Embedded in Terephthalic Acid (TPA)

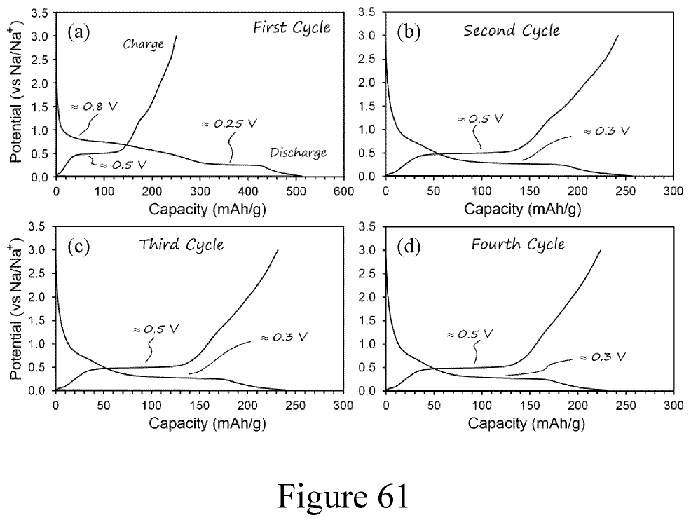

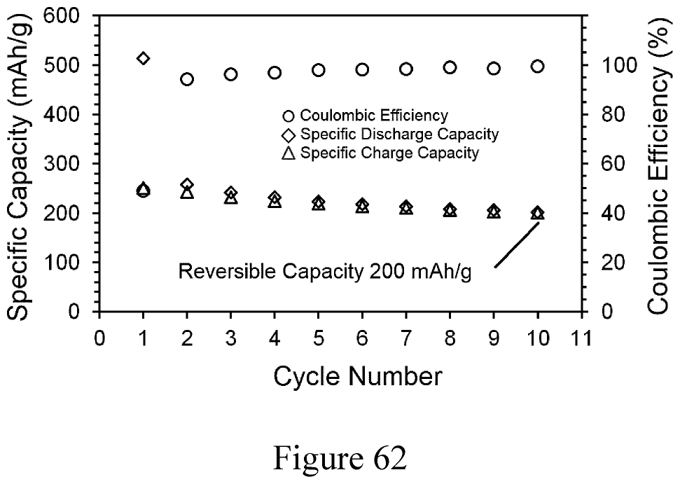

To observe the Na-ion storage performance, an electrode was made using heat-treated Zn-TP embedded in terephthalic acid (as described in Example 31) as the active material. For this, the active material was mixed with conductive carbon (C45) and PVDF binder with the mass ratio of 6:3:1 using NMP as the solvent. The resulting slurry was then coated on copper foil using a 200 m doctor blade, and dried in a vacuum oven at 80° C. for 12 hours. The mass loading of the electrode was around 1.5 mg/cm². Coin-type half-cells (CR 2032) were assembled using metallic sodium as the counter/reference electrode, 1.0 M NaCF₃SO₃ in diglyme as the electrolyte, and glass microfiber (Whatman, 1823025) as the separator. The half-cells were assembled in a glove box under high purity argon with 02 and H₂O contents of less than 0.1 ppm. The cells were allowed to stabilize at room temperature for 10 hours, and then assembled on a battery test system, where the measurements were conducted at a constant current of 30 mA/g in the voltage range 0.01-3.0 V vs Na/Na⁺. The galvanostatic discharge-charge curves were recorded at a current density of 30 mA/g. The discharge/charge profiles for the first four cycles are shown in FIG. 61a-d, respectively. As can be observed, the first discharge curves exhibited plateaus at around 0.8 and 0.25 V vs Na/Na⁺, while the subsequent discharge curves showed plateaus at around 0.3 V. On the other hand, the charge curves were characterized by the presence of well-established plateaus at around 0.5 V vs Na/Na⁺. The discharge plateau at around 0.25 and 0.3 V was well above 0.0 V vs Na/Na⁺ avoiding the formation of metallic Na on the surface of the electrode, thus increasing the safety of the electrode. Also, the charge plateau at around 0.5 V vs Na/Na⁺ was sufficiently low to ensure the high energy density of the battery having the Zn-TP embedded in terephthalic acid as the anode active material. Cycling performance of the electrode recorded at 30 mA/g is shown in FIG. 62, where the reversible capacity of 200 mAh/g was detected after 10 Na-ion insertion/extraction cycles.

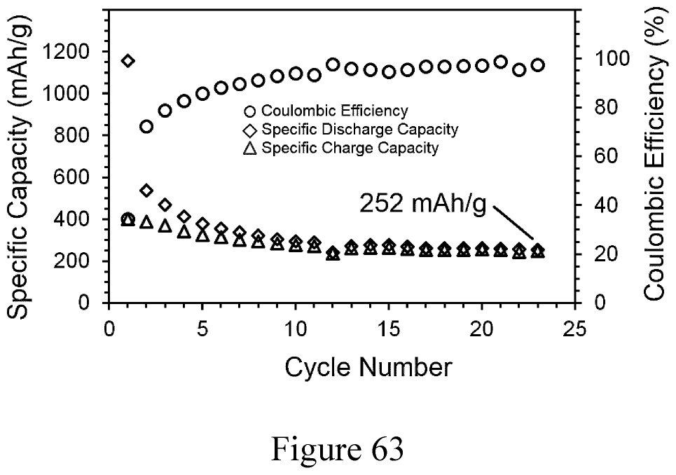

The Li-ion storage performance of heat-treated Zn-TP embedded in terephthalic acid was also examined. Zn-TP embedded in terephthalic acid was mixed with PVDF binder and conductive carbon (Super P) (6:1:3) using NMP as the solvent. The slurry made was coated on copper foil, and dried at 80° C. for 10 h under vacuum. As described in previous examples, coin cells were assembled using Li as the counter/reference electrode and 1.0 M LiPF₆ in EC:DEC:EMC (1:1:1 wt %) as the electrolyte. The electrode obtained with mass loading of around 1.5 mg/cm² was cycled at the current density of 100 mA/g in the potential window 0.01-3 V vs Na/Na⁺. FIG. 63 shows the cycling performance of the electrode. A stable reversible capacity of 252 mAh/g was recorded after 23 Li-ion insertion/extraction cycles.

The results show that heat-treated Zn-TP embedded in terephthalic acid product (obtained in Example 31) can be used as an efficient anode of metal-ion batteries, such as the anode of Na-ion batteries (SIBs) or Li-ion batteries (LIBs).

### Example 34—Treatment of HDPE with SnCl₂

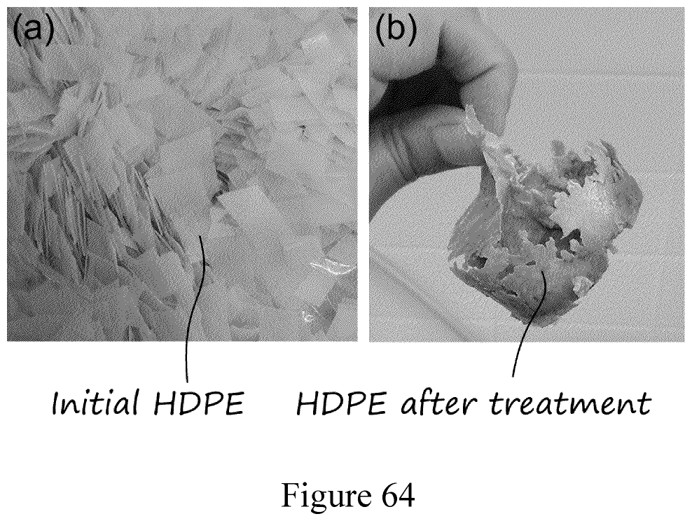

A plastic bottle made of HDPE was cut into pieces with dimensions of a few centimeters. A quantity of 30.0 g HDPE pieces was mixed with 140.0 g SnCl₂ and the mixture (170.0 g) was loaded into an alumina crucible. The crucible was heated in a vertical resistance furnace under flow of argon gas to 337° C. with an average heating rate of 3° C./min, while the temperature inside the crucible was measured throughout the experiment. After termination of heating, the crucible was cooled down to room temperature. The mass of materials after the heat-treatment was measured to be 168.0 g, indicating a negligible mass loss of ˜0.7% which corresponded to the dehydration of the materials during the heat-treatment process. FIG. 64a shows the HPDE initially and FIG. 64b shows the HDPE after the heat treatment. As shown in FIG. 64b, HDPE was not depolymerized during the heat-treatment process, making it easy to collect from the container.

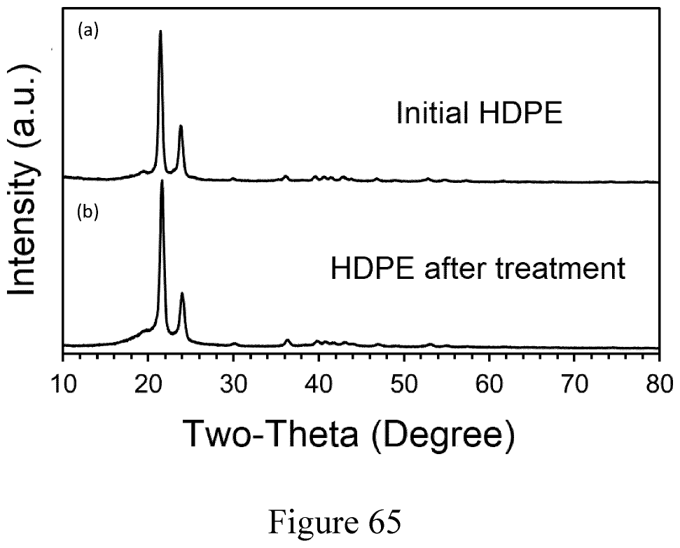

The X-ray diffraction pattern of HDPE after and before heat treatment process is shown in FIGS. 65a and 65b, respectively. The XRD patterns show that the crystalline structure of HDPE remained unchanged during the process.

The results suggest that a mixture of PET with HDPE heated with SnCl₂ to a sufficiently high temperature would depolymerize PET, while HDPE would be melted during the heating process, and sink to the bottom of the container, leaving the porous structure of terephthalic acid monomers, resulting from the depolymerization of PET, on the upper part of the container.

### Example 35—UV-Vis Measurements

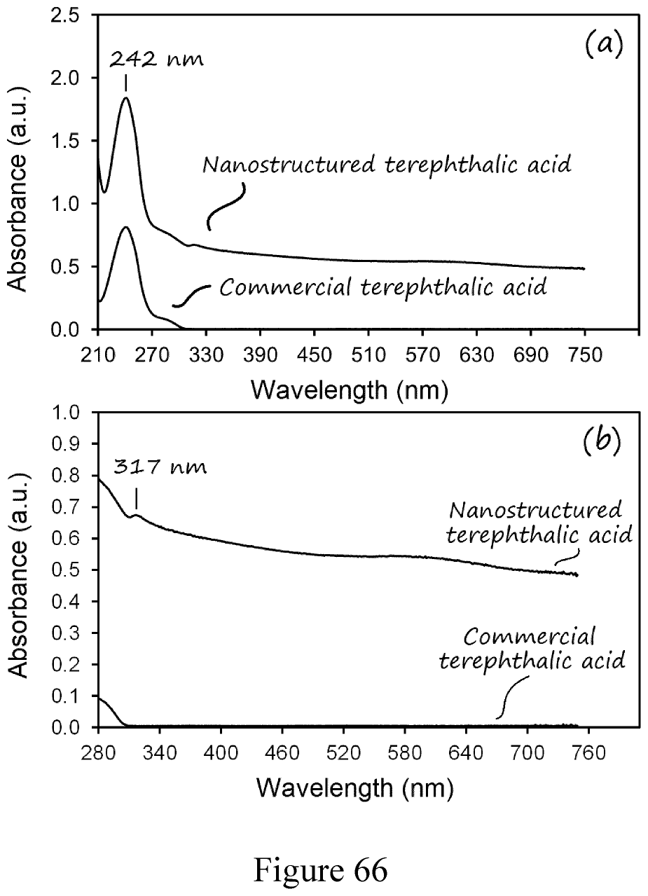

FIG. 66a shows the UV-Vis absorbance spectrum of the nanostructured terephthalic acid (TPA) prepared by the heat-treatment of PET and SnCl₂ (described in Example 21), and commercially available terephthalic acid. To prepare the samples for UV-Vis measurements, the samples were added to deionized water and then subjected to ultrasonication for 10 min prior to UV-Vis measurements using a UV-Vis spectrophotometer (Thermo Scientific Evolution 220). The concentration of TPA materials in deionized water was adjusted to be about 0.5 g/L. Before measuring the absorbance spectra of the TPA materials, the absorbance of deionized water was measured as the baseline. When measuring the absorbance of the TPA materials, the instrument automatically subtracted the absorbance of water.

As can be observed from FIG. 66a, both samples show a peak at approximately same wavelength number; 242 nm for nanostructured terephthalic acid and 241 nm for commercial terephthalic acid, indicating the same crystalline structure of the two compounds.

Despite having this similarity, the two compounds show obvious differences in terms of light absorbance. First, the light absorbance of the nanostructured terephthalic acid at the peak of 242 nm (1.840 a.u.) is around 2.3 times greater than the light absorption of commercial terephthalic acid at the adsorption peak (0.815). Second, the light absorbance of commercial terephthalic acid is close to zero at larger wavenumbers greater than 300 nm. In contrast, the nanostructured terephthalic acid shows a relatively large light absorbance at all wavelengths. For instance, at the concentration of 0.5 g/L, the following absorbance data was observed from FIG. 66a: 450 nm (0.564 a.u.) and 500 nm (0.548 a.u.).

Moreover, FIG. 66b shows that the nanostructured terephthalic acid exhibited an additional peak at 317 nm, which was absent in the commercial terephthalic acid. Without wishing to be bound by theory, this peak can be attributed to the light absorbance feature brought about by the presence of nanoporosities on the surfaces of the nanostructured terephthalic acid.

As expected from FIGS. 28a-b, the appearance of the nanostructured terephthalic acid is much darker than that of commercially available terephthalic acid, easily distinguishable by the human eye.

### Example 36—Production of Na₂TP Using Commercial and Nanostructured TPA

1.5 g NaOH was mixed with 1.5 g TPA (either commercial TPA or nanostructured TPA) in 60 mL ethanol (purity≥99.7%, 0.789-0.791 g/mL at 20° C.) under magnet stirring for various periods of time (6 h, 12 h, 18 h, and 24 h). Then, the mixture was subjected to centrifugation to retrieve the product, which was subsequently dispersed in ethanol and centrifuged again. This last step was repeated two times, and then, the obtained powder was dried under vacuum at 150° C. for 1 h.

X-ray diffraction patterns were recorded on the commercial TPA, the nanostructure TPA and the products obtained at various processing time periods using a PANalyco instrument with Cu—K_{α} radiation (λ=1.54 Å) with step size, dwell time and scan speed of 0.033°, 45 s and 0.094 degree/second, respectively.

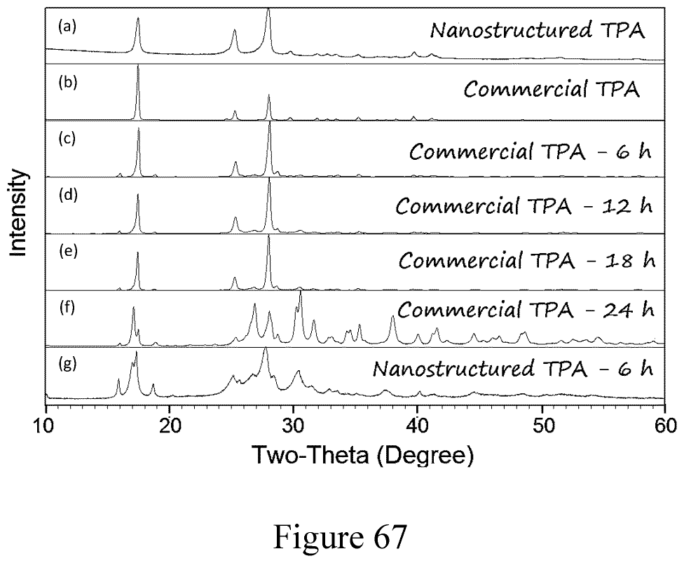

As shown in FIG. 67a, all diffraction peaks of the nanostructured TPA can be assigned to terephthalic acid with anorthic crystalline structure (ICDD #00-021-1919), demonstrating the preparation of nanostructured TPA. Among the diffraction peaks, the peaks with higher values of intensity are located at two-theta values of 17.517° (FWHM=0.276°), 25.301 (FWHM=0.276°), and 28.080° (FWHM=0.335°).

FIG. 67b shows that diffraction peaks of commercially available TPA can be assigned to those of terephthalic acid (ICDD #00-021-1919) with anorthic crystalline structure. Selected XRD peaks of commercial TPA can be observed at the two-theta values of 17.4830 (FWHM=0.138°), 25.291° (FWHM=0.197°), and 28.0190 (FWHM=0.1570). As can be observed, FWHM values for the nanostructured TPA are considerably larger than those of commercial TPA demonstrating the substantially finer crystalline domain sizes in the earlier.

FIGS. 67c-e show that the commercial TPA treated for 6 h, 12 h and 18 h, respectively, does not show any structural change, demonstrating the relatively slow kinetics of the process.

In contrast to the commercial TPA, the nanostructured TPA is considerably more reactive towards NaOH so that only 6 h of treatment (or less) was sufficient to prepare Na₂TP.

### Example 37—Silicon-Thermally Modified PI Electrode for Li-Ion Storage

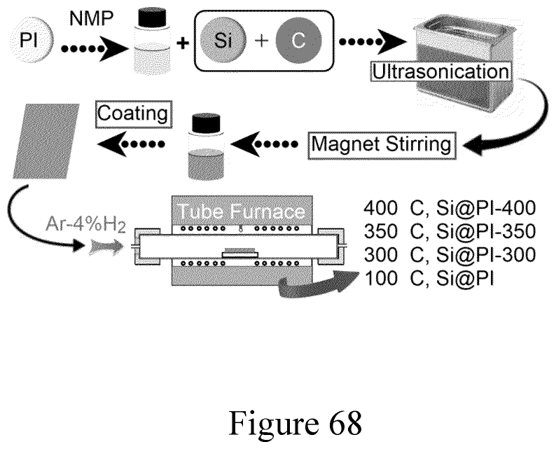

The effect of PI binder on the Li-ion storage performance of SiNPs (particle sizes=20-60 nm, ≥99.9%, Aladdin) was investigated by assembling half-cells. First, 10 mg PI powder (PI, M_{w}=50000-80000, Macklin) was mixed with 405 mg (400 μL) NMP to form a uniform solution. Then, 10 mg conductive carbon (Super P, ≥99.9%) and 80 mg SiNPs were added to the PI solution, and the mixture was subjected to ultrasonic treatment for 2 h to obtain a uniform dispersion. Thereafter, the dispersion was stirred for 3 hours, and the resulting uniform slurry was coated on Cu foil, and dried at room temperature for 10 min, and then at 100° C. for 2 h under vacuum to remove NMP. After drying, the electrode was heated to various temperatures of 300, 350 and 400° C. with a heating rate of 2° C./min under an Ar-4% H₂ stream in a tube furnace, with a dwell time at the maximum temperature of 2 h. The temperature was then reduced to room temperature under the same gas flow, and the electrode obtained was used to assemble a coin cell. FIG. 68 shows a schematic for the process used to prepare the electrodes.

For comparison, electrodes were fabricated using PAA/CMC (1:1 mass ratio) as the binder according to the procedure above, without the high-temperature heat-treatment being applied. The coin cells (2025 type) were assembled in an argon-filled glovebox, using polypropylene as the separator, 1 M lithium hexa-fluorophosphates (LiPF₆) as the electrolyte salt, and the mixture of ethylene carbonate (EC), diethyl carbonate (DEC) and ethyl methyl carbonate (EMC) (EC/DEC/DEC=1:1:1 by mass) as the salt solvent. In the assembled coin cells, metallic lithium discs were used as both the reference and counter electrodes. X-ray diffraction (XRD) phase characterization was conducted using a D8 ADVANCE equipment using Cu-Kα radiation (1.5405 Å) in the range 2θ=10-80°. Fourier transform infrared (FTIR) examination was performed using a VERTEX70 equipment employing KBr pellet as the reference. X-ray photo-electron spectroscopy (XPS) was carried out using a Thermo Scientific K-Alph instrument. UV-Vis absorption spectroscopy was conducted using an Evolution 220 instrument using NMP as the solvent. The morphology of cycled electrodes was studied through scanning electron microscopy (SEM, ZEISS EVO18) after being immersed in dimethyl carbonate (DMC) overnight before SEM measurements. Galvanostatic charge-discharge measurements were performed at 0.01-3.0 V (25° C.) using a LAND battery test system. The electrochemical reactions taking place in the electrodes were evaluated by cyclic voltammetry (CV) performed on an electrochemical workstation (CHI 660E, China) at a scan rate of 0.1 mV s⁻¹ in the voltage range 0.01-3.0 V vs Li/Li⁺. Electrochemical impedance spectroscopy (EIS) measurements were performed employing an AC oscillation amplitude of 5 mV over a frequency range from 100 kHz to 10 MHz. At the stable OCV status, EIS data were measured by the CHI 660E electrochemical workstation.

The PI material used was a thermoplastic polymer formed by the polycondensation and imidization of 1-(4-aminophenyl)-1,3,3-trimethyl-2H-inden-5-amine (DAPI) and benzophenone-3,3′,4,4′-tetra-carboxylic dianhydride (BTDA) and was soluble in NMP.

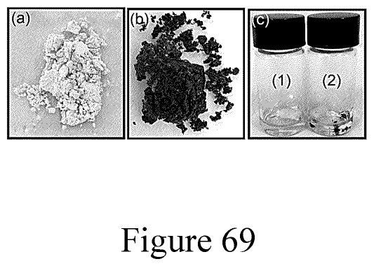

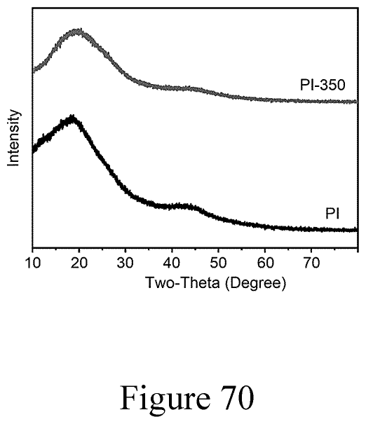

The PI material was heated to 350° C. with a dwell time of 2 h. After the heat-treatment, the PI material underwent an obvious change from light-yellow powder to dark-brown hard lumps. FIGS. 69a and 69b show photographs of the PI before and after heat treatment, respectively. It was observed that the heat treated PI (PI-350) became insoluble in polar solvents as shown in FIG. 69c. Without wishing to be bound by theory, it is believed that this phenomenon can be ascribed to the crosslinking and/or crystallization of the compound. To investigate changes taking place in the crystalline structure of PI, samples before (PI) and after heat treatment (PI-350) were analyzed by X-ray diffraction, and the patterns obtained are shown in FIG. 70.

In the XRD pattern of the PI before heat treatment, two broad diffraction peaks were present at 2θ=10-30° and 2θ=40-50°, which correspond to the amorphous structure of PI, demonstrating the disordered arrangement of the PI molecular chains. Meanwhile, these two broad peaks also appeared in the XRD pattern of PI-350, illustrating the same amorphous structure of PI-350. Selected physical properties of PI before and after heat treatment are presented in Table 6. As can be seen, in addition to differences in appearance and NMP solubility, PI-350 was found to be mechanically much harder in comparison with PI. This may indicate the greater structural packing of the polymer chains in PI-350, with decreased free volume. This property could contribute to higher mechanical properties of PI binder to alleviate volume expansion involved in the cycling of Si electrode as discussed later in this article.

TABLE 6
Selected properties of PI before and after the heat treatment.

| Characteristic | Original PI | PI-350 |
|---|---|---|
| Structure | Amorphous | Amorphous |
| Colour | Light yellow | Dark brown |
| Appearance | Powder | Lump |
| Solubility in NMP | High | Low |

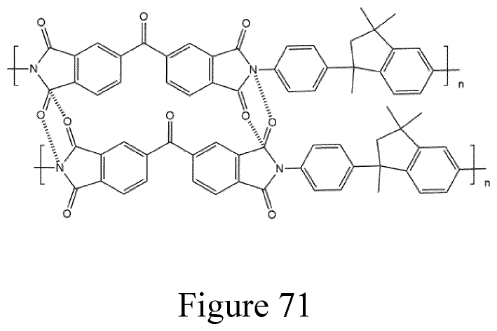

Without wishing to be bound by theory, the occurrence of crosslinking between molecular chains of PI taking place through the heat treatment process may lead to the formation of charge transfer complexes (CTCs), which is in turn responsible for the differences observed between properties of the original PI and those of PI-350. Without wishing to be bound by theory, it is believed that CTCs are formed through electron transfer between the electron-rich donor molecules and electron-deficient acceptor molecules, such as the five-membered imide rings and benzene rings. Without wishing to be bound by theory, it is believed that after heat treatment, the molecular chains of PI are able to approach each other closely enough to allow transfer of p-electron through the electron donator and accepter compartments of PI, leading to the formation of CTCs among PI chains as exhibited in FIG. 71.

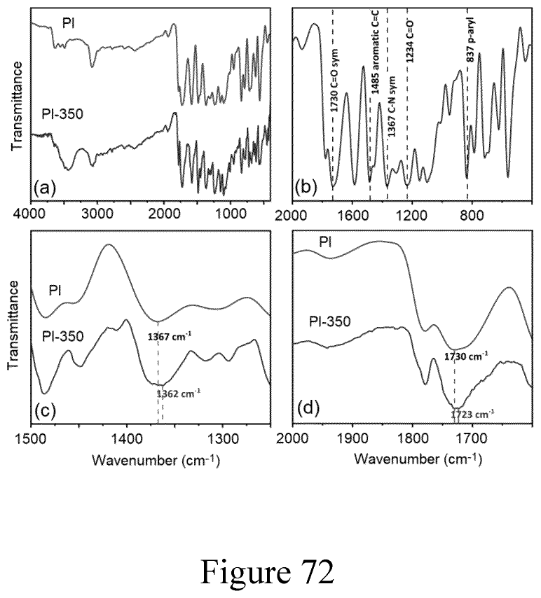

To demonstrate the formation of CTCs in PI during the heat treatment, FTIR spectroscopy was carried out on the original PI and PI-350. The FTIR spectrum of PI (FIGS. 72a and 72b) show bands at around 1778 cm⁻¹ attributed to C═O asymmetric stretch of imide groups. The peak at 1730 cm⁻¹ was attributed to C═O symmetric stretch of imide groups, and the peak at 1367 cm⁻¹ to C—N stretch of imide groups. These three peaks represent the stretch vibration of imide groups, demonstrating the presence of the PI material. Additionally, the peak at around 837 cm⁻¹ was ascribed to the p-aryl of the benzene ring, which is due to the vibration of the unsaturated π bond. The FTIR spectrum of PI-350 is shown in FIG. 72a, in which the peaks corresponding to the symmetric C═O, asymmetric C═O and C—N stretch in imide groups are detectable at around 1778, 1723 and 1362 cm⁻¹, respectively. The presence of these peaks demonstrates that the chemical structure of PI is not affected by the heat treatment process, providing evidence for the thermal stability of PI. The results indicate that, the polymeric structure of PI was preserved during the heat-treatment and is therefore useful as the binder of Si anodes.

By comparing the FTIR spectra of PI with that of PI-350, the peaks related to C═O and C—N bonds in imide groups both shifted to lower wave numbers. Without wishing to be bound by theory, this red-shift phenomenon was caused by the interaction among intermolecular groups. Without wishing to be bound by theory, it is believed that, for the case of PI-350 sample, under the influence of the heat treatment, the molecular chains could approach each other close enough to form CTC structures, through crosslinking, as shown in FIG. 71. Therefore, the peaks related to the C═O bond in imide groups shifted from 1730 to 1723 cm⁻¹ after heat treatment as shown in FIG. 72c. Moreover, the peaks related to C—N bond in imide groups shifted from 1367 to 1362 cm⁻¹. The FTIR results suggest the formation of CTC structure among molecular chains of PI during the heat treatment, and this can explain the differences observed between properties of PI and PI-350 (Table 6). The presence of CTC structure could provide the PI binder with greater packing density, thereby imparting mechanical properties that could improve the electrochemical performance of Si-PI electrodes.

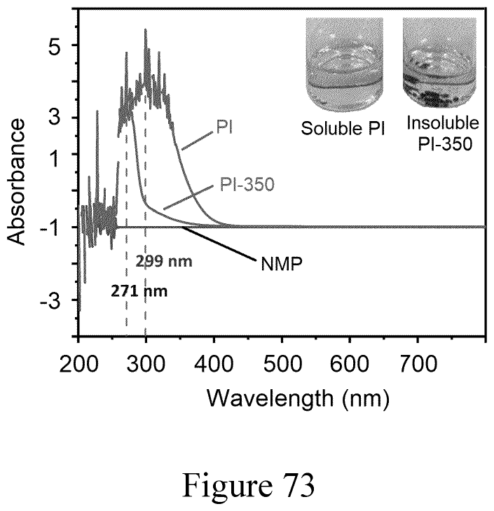

The UV-Vis results recorded on the PI/NMP solution and the upper part of the PI-350/NMP are shown in FIG. 73. Both samples were colourless and transparent due to the dissolution of PI, and the settling of PI-350 particles in NMP media. In the UV-Vis spectrum of NMP, no absorption could be observed. However, the UV-Vis spectra of both PI and PI-350 show the presence of a broad absorption peak at the wavelength 229 and 271 nm, respectively. This peak is attributed to conjugated carbonyl C═O of the benzene ring on the molecular chain of PI. As can be observed, compared with that of PI, the absorption peak of PI-350 is narrower, and has shifted to a lower wavelength value, which can be related to the formation of CTCs among molecular chains of the PI. Therefore, the existence of CTCs in PI-350, brought about by the crosslinking among molecular chains of PI could further be confirmed.

PI material with abundant carbonyl groups (C═O) in imide rings can form strong hydrogen bonding with SiNPs utilizing hydroxyl groups (—OH) on their surface. Without wishing to be bound by theory, it is believed that this allows the PI binder to bond with SiNPs relatively firmly, making a relatively robust electrode with the potential to alleviate volume expansions that occur during the Li⁺ insertion and de-insertion.

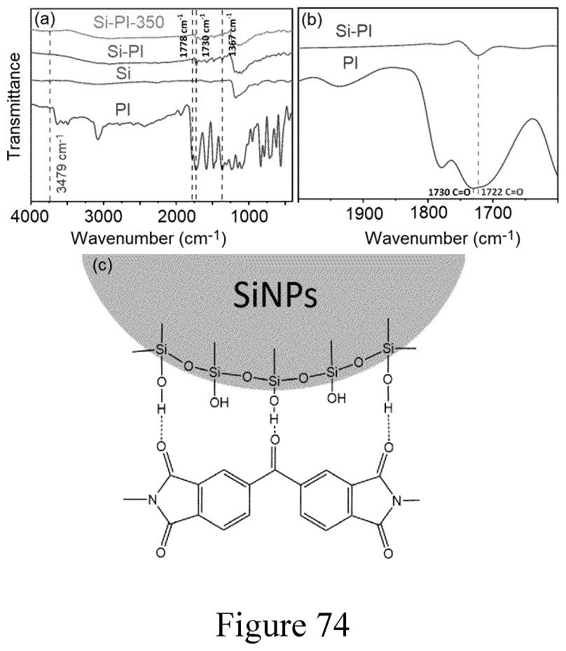

Surface characterizations, including FTIR and XPS were conducted on SiNPs, PI, Si—PI and Si-PI-350. FTIR characterization was used to explore the interaction between the active Si material and the PI binder, as exhibited in FIGS. 74a and 74b. A schematic representation of hydrogen bonding between a SiNP and PI is shown in FIG. 74c. FTIR spectrum of Si-PI and Si-PI-350 can be characterized by the presence of three characteristic peaks at around 1778, 1730 and 1367 cm⁻¹, corresponding to the asymmetric stretch of C═O, and the symmetric stretch of C═O and C—N in imide rings, respectively. The FTIR spectra of Si-PI and Si-PI-350 indicate the existence of PI, due to the presence of the PI characteristic peaks. In the FTIR spectrum of SiNPs, shown in FIG. 74a, the stretch peak related to hydroxyl group (—OH) on surface of nanoparticles appear at the wavenumber of 3739 cm⁻¹. However, this peak is absent in the FTIR spectrum of Si-PI-350 shown in FIG. 74a. Without wishing to be bound by theory, it is believed that this observation can be explained by the interaction of the —OH group on Si with C═O group of PI to form the hydrogen bonding. Besides, the stretch peak corresponding to the C═O group in imide ring of PI shifts from 1730 in the original PI to 1722 cm⁻¹ in the Si-PI as shown in FIG. 74b. The red shift of 8 cm⁻¹ wavenumber can also be due to the formation of hydrogen bonding.

Without wishing to be bound by theory, it is believed that the hydrogen bonding between the surfaces of Si active material and the binder can provide the electrode with a tighter structure, further allowing the electrode to sustain volume changes during the cycling process. On the other hand, in the spectrum of Si-PI-350, the peak related to —OH groups vibration (absent in Si-PI) appeared again at the wavenumber of 3739 cm⁻¹, which can be due to the formation of CTCs functionalized by hydroxyl groups. This can improve the electrochemical performance of the electrode, as explained below.

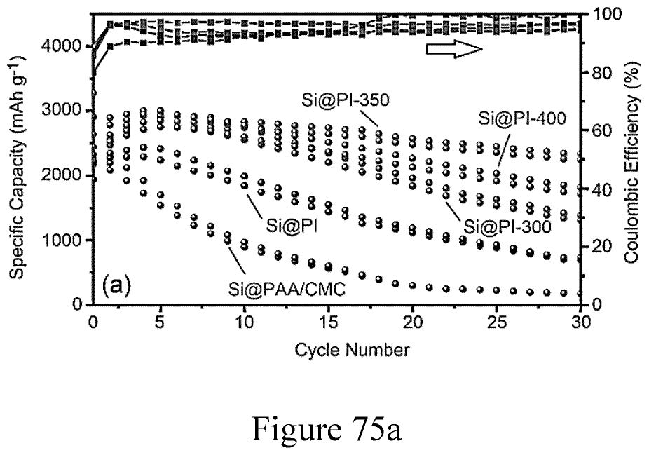

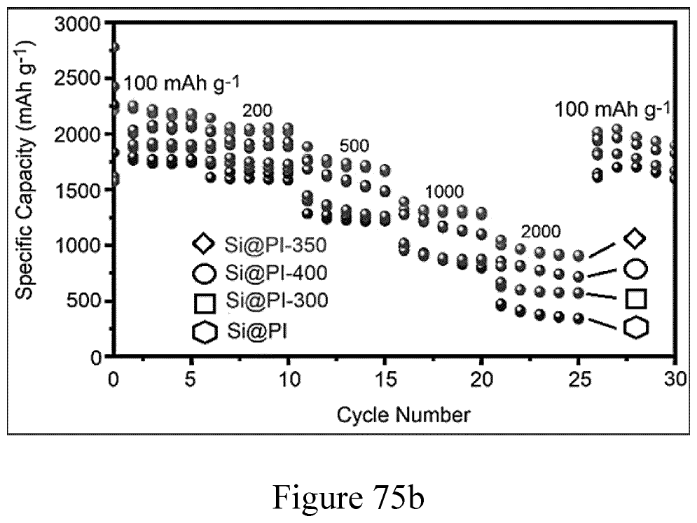

The influence of heat-treatment on the electrochemical performances of electrodes made of SiNPs and PI binder was evaluated. Electrodes were fabricated using SiNPs and PI as the binder (Si@PI). Then the electrodes were heat-treated in a flow of Ar-4% H₂ at various temperatures of 300, 350 and 400° C. to prepare Si@PI-300, Si@PI-350 and Si@PI-400 electrodes, respectively (see FIG. 68). The Li-ion storage performances of the electrodes were evaluated and the results are shown in FIGS. 75a-b. FIG. 75a shows the cycling performance of the electrodes recorded at the current density of 200 mA g⁻¹. For comparison, the electrochemical performance of the electrode made of Si and PAA/CMC is also shown. Furthermore, the rate capabilities of Si@PI-400, Si@PI-350, Si@PI-300 and Si@PI were evaluated at the current densities of 100, 200, 500, 1000 and 2000 mA g⁻¹, as shown in FIG. 75b. In FIG. 75b, for each cycle number, the order of data from highest specific capacity to lowest specific capacity is Si@PI-350, Si@PI-400, Si@PI-300, Si@PI. Table 7 summarizes the Li-ion storage performances of the electrodes.

According to FIG. 75a, the specific charge capacity of Si@PI-400, Si@PI-350, Si@PI-300, Si@PI and Si@PAA/CMC electrodes after 30 cycles were recorded at 1818, 2334, 1383, 737 and 182 mAh g⁻¹ respectively. As can be observed, the electrodes made using PI binder showed considerably improved cycling performance than those made using PAA/CMC binder. Without wishing to be bound by theory, it is believed that this improvement can be due to more favorable mechanical properties of PI, brought about by benzene rings on molecular chains, allowing the electrodes to maintain the electrode's integrity during the cycling. Moreover, it is evident that the electrochemical performance of Si@PI-350 can be superior to certain other electrodes. For instance, the initial coulombic efficiency of the Si@PI-350 electrode (88.57%) is the highest among the electrodes, while all the PI-containing electrodes maintained a coulombic efficiency over 95% in the following cycles. According to FIGS. 75a and b, the Si@PI-350 electrode showed a greater specific capacity and rate capability among electrodes, which can be ascribed to structural modifications that occurred during the heat-treatment process, and particularly, the formation of optimized CTC structure and hydrogen bonding between PI and SiNPs. Without wishing to be bound by theory, these modifications could provide the electrode with greater mechanical properties, allowing the accommodation of volume expansions/contractions involved during the Li-ion insertion/extraction cycles, providing the electrode with greater electrode's integrity during cycling.

TABLE 7
Li-ion storage performance of different electrodes after
30 cycles at the current density of 200 mA g⁻¹.

| | Initial coulombic | Initial | Final |
|---|---|---|---|
| | efficiency | capacity | capacity |
| Electrodes | (%) | (mAh g⁻¹) | (mAh g⁻¹) |
| Si@PI-400 | 85.76 | 2628 | 1818 |
| Si@PI-350 | 88.57 | 2894 | 2334 |
| Si@PI-300 | 88.05 | 2557 | 1383 |
| Si@PI | 87.32 | 2278 | 737 |
| Si@PAA/CMC | 79.99 | 2344 | 176 |

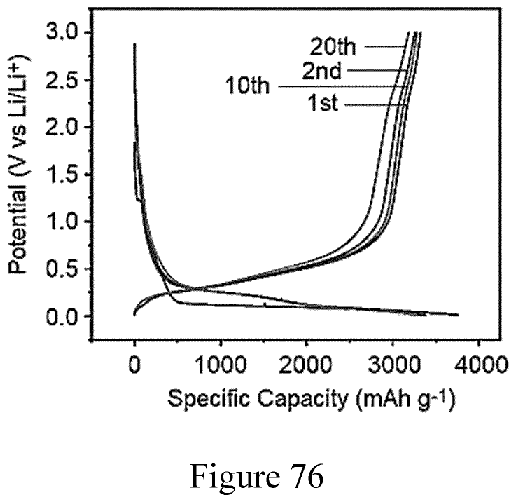

As observed in FIGS. 75a-b and Table 7, the cycling performance of the electrodes was gradually improved by increasing the heat treatment temperature to 350° C., where the peak of electrochemical performance was recorded, indicated by a capacity of 2334 mAh g⁻¹ for Si@PI-350 electrode after 30 cycles at 200 mA g⁻¹. By further increasing the heat-treatment temperature to 400° C., the Li-ion storage performance of the sample (Si@PI-400) degraded, which can be ascribed to damage to the CTC structure and hydrogen bonding at higher temperatures. Without wishing to be bound by theory, an appropriate CTC structure can provide the electrode with relatively high toughness improving its cycling stability and the rate performance as shown in FIG. 75b. Notably, at a current density of 2000 mA g⁻¹ and after 25 cycles, the reversible capacity of Si@PI-350 (900 mAh g⁻¹) was greater than those of Si@PI-400 (713 mAh g⁻¹), Si@PI-300 (569 mAh g⁻¹), and Si@PI (338 mAh g⁻¹). After reducing the current density back to 100 mA g⁻¹, the Si-PI@350 could still exhibit a high reversible capacity of 1898 mAh g⁻¹ after 30 cycles, confirming the robustness of the heat-treated PI binder. FIG. 76 shows the galvanostatic charge-discharge (GCD) profiles of the Si@PI-350 electrode recorded at constant current density of 200 mA g⁻¹. As can be observed, there was a brief plateau at around 1.23 V in the first discharge cycle, which was attributed to the formation of a solid electrolyte interphase (SEI) layer. Subsequent charge/discharge curves showed an excellent consistency, suggesting the desirable cycling performance of Si@PI-350 electrode.

Apart from high-voltage plateau in the first discharge cycle, the Galvanostatic charge-discharge (GCD) curves of the electrode also showed the presence of low-voltage extensive plateaus, which can be assigned to the reactivity of SiNPs in the Li-ion insertion/extraction events.

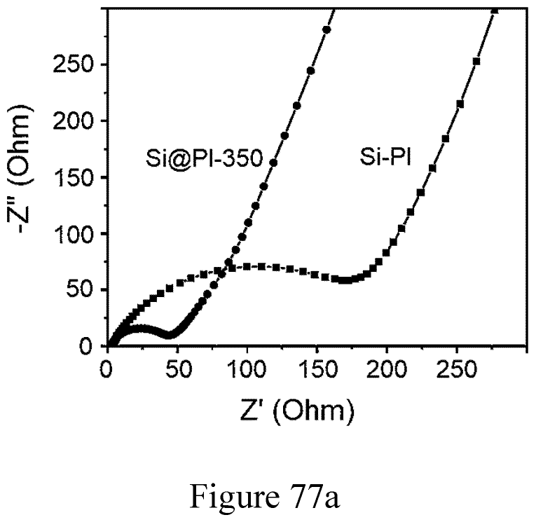

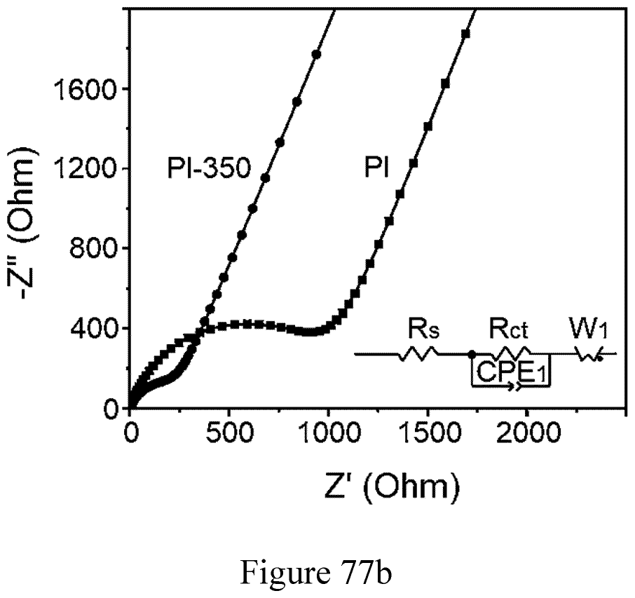

The influence of heat-treatment on the electrochemical resistance of Si@PI electrode was further examined by performing electrochemical impedance spectroscopy (EIS) in the frequency range 0.01-1000000 Hz with AC amplitude of 5 mV. EIS curves of Si@PI and Si@PI-350 electrodes are shown in FIG. 77a, and those of PI and PI-350 in FIG. 77b.

In the EIS curves, the semicircles observed in the high frequency region can be related to the charge transfer resistance (R_{ct}). Moreover, the oblique line in the low frequency region is attributed to the Li-ion diffusion impedance (Rₛ). The smaller diameter of the semicircle corresponds to the smaller electron transfer resistance. The values of impedance extracted from FIGS. 77a and b are shown in Table 8, according to which the smaller R_{ct} value of heat-treated sample is evident. This can mainly be ascribed to the formation of the charge transfer complexes among the main molecular chains of PI and hydrogen bonding during the heat-treatment process, providing a tighter contact within SiNPs, conductive carbon and PI binder.

TABLE 8
Impedance data extracted from FIGS. 77(a) and (b).

| Electrodes | Rs (Ω) | Rct (Ω) |
|---|---|---|
| Si@PI | 2.857 | 130.4 |
| Si@PI-350 | 2.666 | 37.7 |

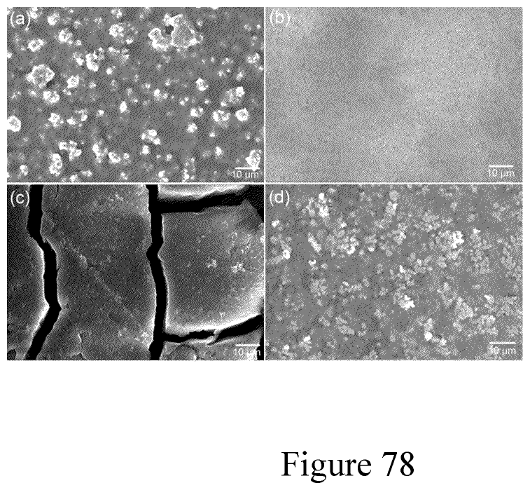

The tighter morphology of heat-treated sample was further confirmed by electron microscopy of Si@PI and Si@PI-350 before cycling and after 20 Li-ion insertion/extraction cycles. SEM micrographs of Si@PI before cycling, Si@PI-350 before cycling, Si@PI after 20 Li-ion insertion/extraction cycles, and Si@PI-350 after 20 Li-ion insertion/extraction cycles are shown in FIGS. 78a-d, respectively.

Cracks with sizes of several micrometers could be observed on the surface of cycled Si@PI electrode, while there were no obvious cracks on the surface of the cycled Si@Pi-350 electrode, which maintained its integrity.

The electrochemical performance of Si@PI-350 anode in terms of specific energy density was evaluated based on the reversible capacity and relative average charge potential of the electrode versus the standard SHE reference of the half-cell. Assuming the reversible capacity and the average charge potential (versus Li⁺/Li) of Si@PI-350 to be 2334 mAh g⁻¹ and 0.53 V, respectively, the value of the average potential (versus SHE) of the electrode was obtained to be 2.54 V, based on the relative potential (−3.04 V versus SHE) of metallic lithium. Therefore, the specific energy density of Si@PI-350 anode was calculated to be 5858 Wh kg⁻¹ at the 30^{th} cycle. As shown, after 30 cycles, the Si@PI-350 electrode exhibited a promising specific capacity and energy density of 2334 mAh g⁻¹ and 5858 Wh kg⁻¹, respectively, outperforming some other binders including sodium carboxymethyl cellulose (CMC), sodium hyaluronate-epichlorohydrin (SH-ECH), polyimine, okra gum, carboxymethyl cellulose-cationic polyacrylamides (CMC-CPAM) and PI with carboxyl group (PI-COOH). In contrast with the complex synthesis methods often employed to prepare binder systems, the preparation of Si@PI-350 electrode system is relatively simple and easily scalable. The observations presented here demonstrate that the implementation of a simple thermal treatment can greatly improve the Li⁺ insertion/extraction performance of Si anodes, due to the formation of CTC structures and hydrogen boding, providing a more compact morphology for the electrode.

### Example 38—Preparation of SnO₂—MoS₂-TPA Nanostructures

2.0 g MoS₂ was mixed with 20.0 g cleaned waste PET pieces, 10.0 g SnCl₂ and 50 g eutectic mixture of LiCl—KCl. The mixture was transferred into an alumina crucible and heated in a resistance furnace in air with the heating rate of 5° C./min to various temperatures in the range 400-600° C. with a dwell-time at maximum temperature of 20 min. Then, the temperature was cooled to room temperature, and the materials obtained were washed with deionized water to remove the soluble components of the product, followed by drying at 100° C. for 2 h.

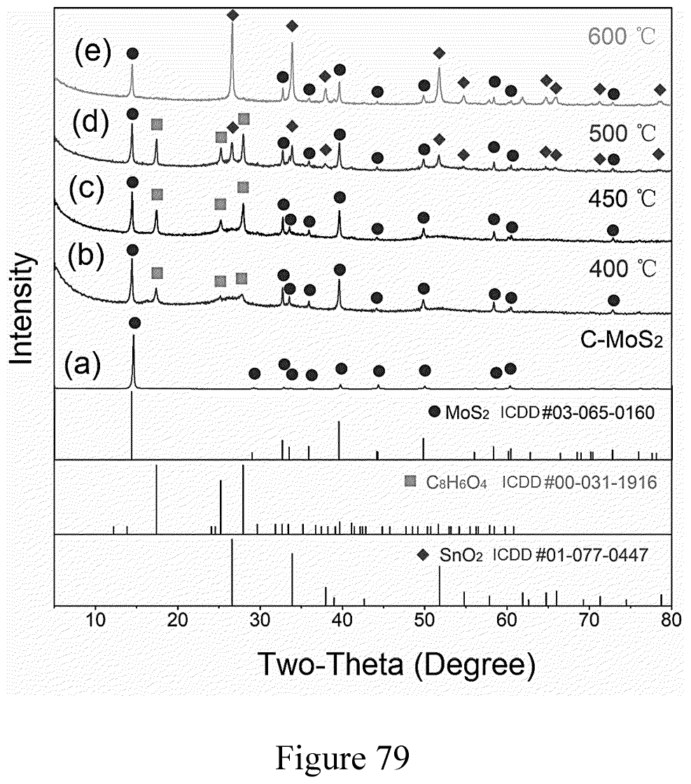

FIG. 79 shows that XRD patterns of the initial MoS₂, and the products obtained at different temperatures, combined with standard XRD patterns of MoS₂, terephthalic acid and SnO₂. As can be observed, the material prepared at 400° C. (FIG. 79b) mainly contained MoS₂ and terephthalic acid. By increasing the temperature to 450° C. (FIG. 79c), the amount of terephthalic acid increased, characterized by relatively more intense peaks of the organic compound in this sample compared to the sample prepared at 400° C. At 500° C., in addition to MoS₂ and terephthalic acid, the diffraction peaks related to SnO₂ can also be observed, as shown in FIG. 79(d). At 600° C., the XRD peaks corresponding to the terephthalic acid disappears, as exhibited in FIG. 79(e).

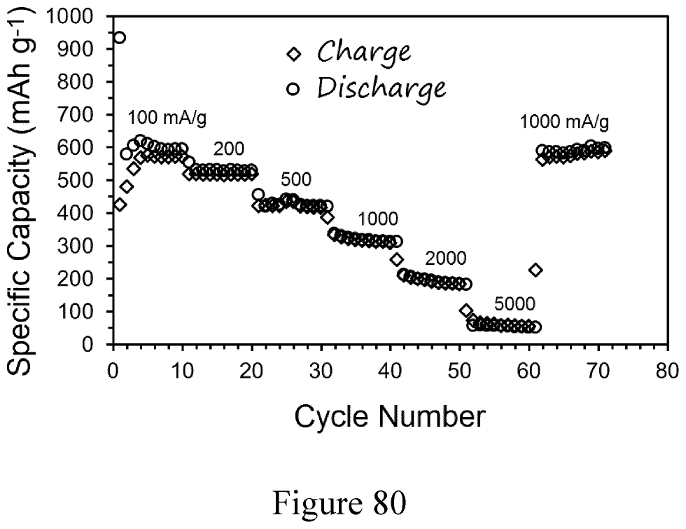

The composite material made at 500° C. containing MoS₂, SnO₂ and C₈H₆O₄ was used to fabricate electrodes for Li-ion storage and was tested at various rates at the voltage range 3.0-0.01 V vs Li⁺/Li. The electrodes were made using the composite material, conductive carbon (C45), PVDF with the mass ratio of 7:2:1, and NMP as the solvent, obtained a mass loading of around 1.2 mg/cm². According to FIG. 80, the electrode delivered a charge capacity of 570 mAh/g after 10 cycles at a current density of 100 mA/g, a charge capacity of 518 mAh/g after 20 cycles at 200 mA/g, a charge capacity of 416 mAh/g after 30 cycles at 500 mA/g, a charge capacity of 309 mAh/g after 40 cycles at 1000 mA/g, a charge capacity of 183 mAh/g after 50 cycles at 2000 mA/g, and a charge capacity of 55 mAh/g after 60 cycles at 5000 mA/g. Upon returning the current density back to 100 mA/g, a charge capacity of 589 mAh/g was recorded at 71th cycle. The results demonstrate the high rate capability and specific capacity of the electrode.

### Example 39—Silicon-Thermally Modified Polyimide-TPA Electrode for Li-Ion Storage

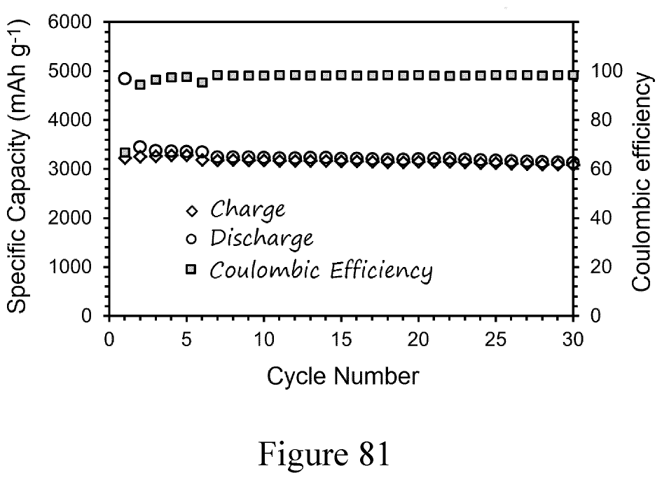

The process explained in Example 27 was repeated with the difference that the mixture used to make slurry for coating on Cu foil contained Si nanoparticles:polyimide:nanostructured TPA:conductive carbon with the mass ratio of 5:2:2:1; and the heat-treatment was performed at the target temperature of 250° C. The nanostructured TPA was obtained based on the process explained in Example 21. The cycling performance of the electrode obtained in half-cell coin cell configuration (2025 type) against lithium at the current density of 200 mAh/g and the cut-off voltage of 0.01-1.5 V is shown in FIG. 81. The values of capacity are reported based on the mass of silicon used in the electrode. The first discharge and charge capacities were recorded at 4836 and 3225 mAh/g, respectively, corresponding to a coulombic efficiency of 66.7%. The second discharge and charge capacity were recorded at 3445 and 3253 mAh/g, respectively indicating a coulombic efficiency of 94.4%. The third discharge and charge capacity were recorded at 3373 and 3256 mAh/g, respectively, indicating a coulombic efficiency of 96.5%. Moreover, the third discharge and charge capacity were recorded at 3373 and 3256 mAh/g, respectively, indicating a coulombic efficiency of 96.5%. Moreover, the values of discharge and charge capacity at cycle number 30 were recorded at 3132 and 3078 mAh/g, respectively, corresponding to a coulombic efficiency of 98.3%. By considering the average charge voltage of 0.5 V, the specific energy density of the electrode at the 30^{th} cycle was evaluated to be 7818 Wh kg⁻¹.

### OTHER EMBODIMENTS

While certain embodiments have been disclosed above, the disclosure is not limited to such embodiments.

As an example, in certain embodiments, the depolymerization agent can contain an ionic liquid in addition to or instead of an inorganic salt. Examples of ionic liquids include [bmpy][Tf₂N] and [BMIM][Tf₂N] and Imidazolium ionic liquids. Examples of cations in the ionic liquid include 1-octyl-3-methyl-imidazolium ([OMIM]), 1-methyl-imidazolium ([MIM]), 1-ethyl-3-methyl-imidazolium ([EMIM]), 1,3-dimethyl-imidazolium ([M131M]), 1-(2-hydroxylethyl)-3-methylimidazolium ([HOEMIm]), 1-ethyl-2,3-dimethyl-imidazolium ([EMMIM]), 1-butyl-3-methyl-imidazolium ([BMIM]), 1-hexyl-3-methyl-imidazolium ([HMIM]), 1,2,3-trimethyl-imidazolium ([MVIMIM]), 1-decyl-3-methyl-imidazolium ([DMIM]), 1-allyl-3-butyl-imidazolium ([ABIM]), 1,2-dimethyl-imidazolium ([M12JM]), 1-butyl-2,3-dimethyl-imidazolium ([BMMIM]), 1-allyl-3-methyl-imidazolium ([AMIM]), 1-allyl-3-vinyl-imidazolium ([AVIM]), tetradecyltrihexylphosphonium ([P66614]), N-ethyl-pyridinium ([EPy]), and N-butyl-pyridinium ([BPy]). Examples of anions in the ionic liquid include bis(trifluoromethylsulfonyl)imide ([Tf₂N]), bromide ([Br]), dicyanamide ([DCA]), hexafluorophosphate ([PF₆]), perchlorate ([CO₄]), tosylate ([TS]), acetate ([Ac]), chloride ([Cl]), glycinate ([Gly]), iodide ([I]), trifluoromethanesulfonate ([TFO]), prolinate ([Pro]), alaninate ([Ala]), lysinate ([Lys]), dihydrogen phosphate ([H₂PO₄]), nitrate ([NO₃]), serinate ([Ser]), glutamate ([Glu]), and hydrogen sulfate ([HsO₄]), and tetrafluoroborate ([BF₄]).

1-438. (canceled)

439. A method, comprising:
heating a mixture comprising reactants to a first temperature; and
forming a composition comprising a nanocrystalline organic compound,
wherein the reactants comprise a polymer and a depolymerization agent.

440. The method of claim 439, wherein the reactants further comprise a salt and heating the mixture makes the salt a molten salt.

441. The method of claim 440, wherein the salt comprises a chloride salt.

442. The method of claim 440, wherein the salt comprises at least one member selected from the group consisting of LiCl and KCl.

443. The method of claim 440, wherein the salt has a melting point of from 250° C. to 700° C.

444. The method of claim 439, wherein heating the mixture depolymerizes the polymer to provide monomers, and the monomers form the organic compound.

445. The method of claim 439, wherein the reactants comprise from 1 weight percent (wt. %) to 99 wt. % of the polymer.

446. The method of claim 439, wherein the polymer comprises at least one member selected from the group consisting of polyethylene terephthalate (PET), poly(acrylonitrile), poly(6-aminocaproic acid), poly(caprolactam), nylon, poly(etheretherketone) (PEEK), poly(ethylene) (PE), poly(hexamethylene adipamide), poly(methyl methacrylate), poly(methylene oxide), poly(4-methylpentene), poly(propylene), poly(styrene), poly(trans-1,4-butadiene), poly(vinyl alcohol), poly(vinyl chloride) (PVC), poly(vinyl fluoride), poly(vinylidene chloride), and poly(vinylidene fluoride).

447. The method of claim 439, wherein a difference in melting temperatures of the polymer and the depolymerization agent is less than 100° C.

448. The method of claim 439, wherein the reactants comprise from 1 wt. % to 95 wt. % of the depolymerization agent.

449. The method of claim 439, wherein the depolymerization agent comprises an inorganic salt.

450. The method of claim 439, wherein the depolymerization agent comprises at least one member selected from the group consisting of tin(II) chloride (SnCl₂), zinc chloride (ZnCl₂), calcium chloride (CaCl₂)), lead chloride (PbCl₂), sodium chloride (NaCl), potassium chloride (KCl), and iron chloride (FeCl₂).

451. The method of claim 439, wherein the heating is performed at a temperature greater than a melting point of the polymer and a melting point of the depolymerization agent.

452. The method of claim 439, wherein the heating is performed at a temperature lower than a carbonization temperature of the polymer and a decomposition temperature of the organic compound.

453. The method of claim 439, wherein the first temperature is from 200° C. to 600° C.

454. The method of claim 439, wherein the mixture is held at the first temperature for 0.01 minutes to 120 minutes.

455. The method of claim 439, further comprising contacting the composition with a solvent.

456. The method of claim 455, wherein the solvent comprises at least one member selected from the group consisting of an aqueous solution, an alkali aqueous solution, an acidic aqueous solution, and a polar organic liquid.

457. The method of claim 455, wherein the composition comprises the depolymerization agent and contacting the composition with the solvent removes at least a portion of the depolymerization agent.

458. The method of claim 455, wherein the composition comprises depolymerization agent and contacting the composition with the solvent hydrolyzes at least a portion of the depolymerization agent.

459. The method of claim 458, wherein:
the depolymerization agent comprises SnCl₂;
the hydrolysis of SnCl₂ forms a second material comprising tin, chlorine, hydrogen, and oxygen; and
the contacting forms a product comprising the composition and the second material dispersed within the organic compound of the composition.

460. The method of claim 439, wherein the organic compound has the formula CₓO_{y}H_{z}.

461. The method of claim 460, wherein:
x is from 2 to 12;
y is from 2 to 8; and
z is from 2 to 14.

462. The method of claim 439, wherein the organic compound comprises at least one member selected from the group consisting of terephthalic acid, terephthalate, dimethyl terephthalate, bis(2-hydroxyethyl) terephthalate, ethylene glycol, phthalic acid, protocatechuic acid, and isophthalic acid.

463. The method of claim 439, wherein heating is performed at a pressure in the range from 0.01 atm to 100 atm.

464. The method of claim 439, wherein the composition further comprises a metal-organic framework embedded within the organic compound.

465. The method of claim 464, wherein the metal-organic framework comprises the organic compound and a metal.

466. The method of claim 439, wherein the reactants are free from acids, bases, and enzymes.

467. The method of claim 439, wherein the method does not include a separation step.

468. The method of claim 439, wherein the heating forms a second organic compound, and the second organic compound is evaporated.

469. The method of claim 439, further comprising reacting the composition with a metal hydroxide to form a product comprising the composition and a metal of the hydroxide,
wherein the reacting is performed for at most 18 hours.

470. The method of claim 469, wherein the product comprises the molecular formula Na₂C₈H₄O₄, Li₂C₈H₄O₄, K₂C₈H₄O₄, or ZnC₈H₆O₄.

471. The method of claim 439, wherein the composition comprises:
a nanostructured organic compound comprising a plurality of molecules having the formula CₓO_{y}H_{z}, wherein:
x is from 2 to 12;
y is from 2 to 8; and
z is from 2 to 14.

472. The method of claim 440, wherein the composition comprises:
an organic compound; and
a crystalline metal oxide,
wherein:
the crystalline metal oxide is distributed within the organic compound.

473. The method of claim 439, wherein the composition comprises crystalline domain sizes of 1 nm to 100 nm.

474. The method of claim 439, wherein a component of the composition has a maximum dimension in at least one dimension below 100 nm.

475. The method of claim 439, wherein the composition further comprises at least one member selected from the group consisting of a metal oxide, a metal, a metal-organic framework, a silicon-containing material, and a graphene-containing material embedded within the nanocrystalline organic compound.

476. A method, comprising:
heating a mixture comprising reactants to a first temperature; and
forming a composition comprising an organic compound and a crystalline metal oxide,
wherein:
the organic compound is at least partially crystalline;
the reactants comprise a salt, a polymer and a depolymerization agent; and
heating the salt makes the salt a molten salt.

477. A method comprising:
heating a mixture comprising a first polymer, a second polymer and a depolymerization agent to a first temperature;
depolymerizing the first polymer to form an organic compound; and
forming a composition comprising the organic compound,
wherein the second polymer is not substantially depolymerized.

478. The method of claim 477, wherein the first polymer is a polymer that can be depolymerized in the presence of water.

479. The method of claim 477, wherein the first polymer comprises at least one member selected from the group consisting of polyethylene terephthalate (PET), polystyrene, polyvinyl chloride, nylon, a polyurethane, a phenolic resin, and an epoxy resin.

480. The method of claim 477, wherein the second polymer comprises at least one member selected from the group consisting of a polyethylene and a polypropylene.
